(19) United States
(12) Patent Application Publication
(10) Pub. No.: US 2025/0018205 A1
(43) Pub. Date: Jan. 16, 2025

(54) Synthesis and Applications of Porosity-based Semiconductor Heterojunctions

(71) Applicant: The University of Chicago, Chicago, IL (US)

(72) Inventors: Bozhi Tian, Chicago, IL (US); Aleksander Prominski, Chicago, IL (US)

(21) Appl. No.: 18/712,154

(22) PCT Filed: Nov. 21, 2022
(86) PCT No.: PCT/US2022/050607

§ 371 Date: May 21, 2024

Related U.S. Application Data
(60) Provisional application No. 63/282,955, filed on Nov. 24, 2021.

(51) Int. Cl. A61N 1/378; A61N 1/362; F03G 7/00

(52) U.S. Cl. CPC A61N 1/3787; A61N 1/362; F03G 7/012

(57) ABSTRACT

The present disclosure relates to semiconductor heterojunctions incorporating porous semiconductor materials. In one aspect, the present disclosure provides a device comprising a p-type semiconductor material comprising a nanoporous semiconductor layer and a nonporous semiconductor layer that form a heterojunction; and a flexible substrate comprising one or more of polymers on which the p-type semiconductor material is distributed such that the flexible substrate is in contact with the nonporous semiconductor layer.

### CROSS-REFERENCE TO RELATED APPLICATIONS

This application is a section 371 U.S. national phase application of PCT/US2022/050607, filed Nov. 22, 2022, which claims the benefit of priority of U.S. Provisional Application No. 63/282,955, filed Nov. 24, 2021, both applications are incorporated herein by reference in]their entirety.

### STATEMENT REGARDING FEDERALLY SPONSORED RESEARCH OR DEVELOPMENT

This invention was made with government support under 2011854, and 2105321 awarded by the National Science Foundation, and N00014-16-1-2958 awarded by the Office of Naval Research. The government has certain rights in the invention.

### FIELD

The present disclosure provides devices comprising porosity-based semiconductor heterojunctions, as well as methods of making and using them, for example for tissue biomodulation.

### BACKGROUND OF THE DISCLOSURE

Flexible bioelectronic devices are extensively employed in bioengineering applications, including drug delivery¹, physiology sensing¹⁻⁴, and bioelectrical modulation^{1-3,5}. Advances in this area have almost eliminated the challenge of biomechanical mismatch⁶; however, the leads of most flexible bioelectronic devices are still associated with potential complications, such as infection⁷⁻⁹, thrombosis^{8,10}, and anatomical position limitations^{11,12} Thus, extensive effort has been invested in the development of leadless biomodulation techniques. Pulsed infrared lasers have been utilized to induce a photothermal effect, which allows neuronal^{13,14} and cardiac^{15,16} tissue modulation; however, the required high-power densities and the infrared laser absorbance by water hinders therapeutic implementation. As an alternative, optogenetics has been successfully employed for neuromodulation^{17,18} and cardiac pacing¹⁹ in vivo, but the need for genetic manipulation would limit its clinical translation. Leadless bioresorbable device used magnetic induction to perform neural regenerative medicine²⁰ and cardiac pacing²¹, while ultrasonic transducers enabled leadless neuromodulation²². Another approach to leadless modulation involves the application of semiconductor materials that can harvest and convert light into an electrical or optoelectronic stimulus²³⁻²⁵. Most of these designs rely on a photodiode configuration in which either p-n or p-i-n junctions are created by doping variation in successive layers. Further, to generate sufficient photocurrents, the surface of the silicon p-i-n junction must be decorated with metals and necessitates high-power densities of light⁵, which limits their therapeutic utility.

### SUMMARY OF THE DISCLOSURE

The present disclosure presents a fast and efficient method for obtaining pure-semiconductor porosity-based heterojunctions with strong photoelectrochemical properties by combining stain etching and high-power oxygen plasma treatment. This method allows a simple and straightforward fabrication of optoelectronic devices out of bulk semiconductors (e.g., silicon), a process that thus far had required extensive and sophisticated instrumentation as in bottom-up (for example, chemical vapor deposition) or top-down fabrication approaches. To demonstrate the utility of such materials in optically induced biomodulation, flexible crystalline semiconductor membranes were fabricated that transduce light pulses with low optical power densities to allow applications such as overdrive heart pacing and nerve bundle activation leading to skeletal muscle contraction. Besides the lack of genetic modification, another advantage over optogenetics is the ability of these devices to modulate the tissues with tissue-penetrating near-infrared (NIR) light, suggesting their potential in diverse therapeutic applications.

In one aspect, the present disclosure provides for a device comprising a p-type semiconductor material comprising a nanoporous semiconductor layer and a nonporous semiconductor layer that form a heterojunction; and a flexible substrate comprising one or more of polymers on which the p-type semiconductor material is distributed such that the flexible substrate is in contact with the nonporous semiconductor layer.

In another aspect, the present disclosure provides for a method for modulating activity of single cells, the method comprising: contacting the cell membrane with the device as otherwise described herein to form a device-cell membrane interface; and exposing the interface to light under conditions to depolarize the cell membrane thereby increase a threshold for activation of the cell, wherein the cell is capable of being activated by photoelectrochemical effect.

In another aspect, the tissue can be engineered tissue. The present disclosure provides for a method for modulating activity of engineered tissues, the method comprising: contacting the engineered tissue surface with the device as otherwise described herein to form a device-engineered tissue interface; and exposing the interface to light under conditions to depolarize the cell membrane thereby increase a threshold for activation of the engineered tissues, wherein the engineered tissues are capable of being activated by light.

In another aspect, the present disclosure provides for a method of treating a disease in a subject by modulating activation of a cell, the method comprising: providing one or more devices as otherwise described herein to the affected cell(s) in the subject; and exposing the one or more devices to the light under conditions sufficient to overcome a threshold for activation of the cell(s) and treat the disease.

In another aspect, the present disclosure provides for a method for photoelectrochemically training myocardium in a subject to beat at a target frequency, the method comprising: contacting the myocardium with one or more devices as otherwise described herein; and operating a light emitter to provide, during a training period of time, a plurality of light pulses to the myocardium at the target frequency.

In another aspect, the present disclosure provides for a method for photoelectrochemically controlling limb movement in a subject, the method comprising: contacting sciatic nerve with one or more devices as otherwise described herein; and operating a light emitter to provide, during a period of time, a plurality of light pulses to the sciatic nerve at the target frequency.

In another aspect, the present disclosure provides for a system for treating a disease in a subject by modulating activation of a cell, the system comprising: one or more devices as otherwise described herein; a light emitter configured to provide a light pulse to the device, wherein the one or more devices provide, to the cell they are in contact with, excitatory stimulus in response to receiving the light; and a controller that is operably coupled to the light source, wherein the controller comprises one or more processors, wherein the controller is programmed to perform controller operations including: operating the light source to provide the light pulse to the cell.

In another aspect, the present disclosure provides for a device comprising a p-type semiconductor material comprising a nanoporous semiconductor layer and a nonporous semiconductor layer that form a heterojunction. For example, in various embodiments, the semiconductor material is silicon.

Other aspects of the disclosure will be apparent to those skilled in the art in view of the description that follows.

### BRIEF DESCRIPTION OF THE DRAWINGS

The accompanying drawings are included to provide a further understanding of the systems and methods of the disclosure, and are incorporated in and constitute a part of this specification. The drawings are not necessarily to scale, and sizes of various elements may be distorted for clarity. The drawings illustrate one or more embodiment(s) of the disclosure and, together with the description, serve to explain the principles and operation of the disclosure.

FIG. 1: Nanoporous/non-porous silicon materials enable efficient photoelectrochemical effects and their biomimetic structure makes them suitable for application in biointerfaces. (a) Diodes are the key building blocks for solar cells and photoelectrochemical cells. The p-n junction creates built-in electrical field that separates the light-generated electrons and holes. In a photocathodic reaction, the electrons can reach the surface of an n-type silicon for reduction reactions. A difference in porosity can create a diode-like band alignment in a p-type silicon. This heterojunction demonstrates strong photoelectrochemical properties without the need for dopant modulation. Moreover, the porous surface yields a softer biointerface, that may further reduce the biomechanical mismatch. (b) Thin and flexible nanoporous/non-porous silicon membranes allow stimulation of rat hearts ex vivo and sciatic nerves in vivo using low energy light pulses.

FIG. 2: Microscopy analysis of the material structure. (a) Scanning electron microscope image shows porosification of the material surface and formation of microscale pillars. Inset shows porous surface. (b) Three-dimensional reconstruction of the material surface from atomic force microscopy (AFM) scan shows that the height of the pillar-like structures is hundreds of nanometers. Results (b-c) are representative of over three independent experiments. (c) Formation of micropillars can be attributed to the coalescence of hydrogen bubbles during wet etching. Schematic presents the proposed model of self-masking leading to pillar formation. (d) Scanning transmission electron microscopy (STEM) image shows the interface between the non-porous and nanoporous silicon in the material. Selected area electron diffraction patterns taken on different material domains show the same crystalline diffraction pattern and no signs of amorphization. (e) High-magnification STEM images show crystallinity in the nanoporous region. Characterisation was performed on samples etched for 1 min in 1% HNO₃ in HF (v/v). Single focused ion beam section of the material was prepared and imaged with STEM in this study. (f) Elastic modulus of nanoporous silicon samples. Three measurements on two samples were performed for Nₘₑₐₛ=6. Boxes bind interquartile range (IQR) divided by the median; whiskers extend 1.5±IQR. All datapoints are plotted. Statistics are calculated using Student's t-test, two-sided. p-values for comparisons are shown: * p<0.001. All datapoints are plotted.

FIG. 3: Screening of etching conditions for photocurrent generation. (a) Schematic of the patch-clamp integrated photocurrent measurement setup used to study wafers and membranes. (b) Definition of the total injected charge, capacitive charge, and faradaic charge, used as figures of merit in optimisation of material processing parameters. (c) Photocurrents measured from p-type silicon materials under different etching time with 1% nitric acid. Even short etching times (10 s) are sufficient to generate strong photocurrents from the materials. Oxygen plasma induces photocurrent enhancement across the entire series and recorded photocurrents show similar saturation levels. Large photocurrents are also recorded with 2 μm silicon membranes. 10 ms, ˜4.3 W/cm², 532 nm LED pulses are used (power: 19 mW, spot size 0.75 mm). Boxes bind interquartile range (IQR) divided by the median; whiskers extend 1.5±IQR. All datapoints are plotted. Each box is calculated from at least three different locations on four separate samples for Nₘₑₐₛ≥12. Experimental groups are paired such as photocurrents from the same sample are measured before and after oxygen plasma treatment. Statistics are calculated using Tukey HSD. p-values for comparisons are shown: * p<0.05; ** p<0.001, all none significant (n.s.) comparisons had a p_value>0.8. (d) Representative photocurrent traces recorded from the stain etched wafers.

FIG. 4: Electrochemical analysis of the nanoporous silicon. (a) Cyclic voltammetry of silicon electrodes in the dark shows improved pseudocapacitive performance of the nanoporous (por-Si) and activated silicon. 0.5 M K₂SO₄, pH 6.3. (b) Under light illumination, reversible redox peaks are identifiable in por-silicon, which can be attributed to oxygen reduction reaction on the surface of the material. Black arrows indicate reversible redox peaks in por-Si, and red arrows indicate irreversible redox peaks in a gold-decorated p-i-n junction. (c) Electrochemical impedance spectroscopy of silicon electrodes shows that stain etching combined with oxygen plasma treatment reduces impedance in the entire range of frequencies. Two electrodes of each type were fabricated, and representative results are shown.

FIG. 5: Pacing of isolated hearts ex vivo. (a) Schematic of experimental setup used for isolated heart in the Langendorff apparatus. (b) Photograph of the membrane attached to the heart on the left ventricular wall. (c) Photograph of 532 nm laser illuminating membrane attached to the isolated heart. (d) Synchronized electrocardiogram (ECG) and transistor-transistor (TTL) signals showing heart stimulation at 4 Hz using 532 nm laser with sub-(2.4 mW/mm²) and supra-threshold (4.0 mW/mm²) optical power densities. Subthreshold stimulation results in only small artifacts in the ECG trace; suprathreshold illumination demonstrates overdrive pacing. (e) When the heart is stimulated using 808 nm lasers at 4 Hz, the threshold is higher, as only 8.7 mW/mm² is able to achieve overdrive pacing. Results in (d) and (e) are representative of over four independent experiments performed with different membranes and hearts. (f) Optical power threshold for stimulation of heart tissue with 532 and 808 nm lasers. Central point represents the lowest power setting for which uninterrupted pacing was observed. Error bars represent half of the distance to the nearest laser power setting to estimate power readout limitations. (g) Photography of fiber setup and microelectrode array used in the dual chamber pacing experiments. 532 nm laser (4 mW/mm²) was used to stimulate LV and 808 nm laser (60 mW/mm²) was used to stimulate RV. (h) Isochrone maps of the electrical propagation in the ex vivo heart show different patterns of electrical propagation for spontaneous, single chamber (LV or RV), or dual chamber optical pacing.

FIG. 6: In vivo sciatic nerve stimulation. (a) Schematic diagram of sciatic nerve stimulation in an acute in vivo rat model. (b) Photograph of silicon membrane wrapped around the sciatic nerve. (c) Still images from the video before (left) and after (right) photo-stimulation showing limb movement. (d) Analysis of nerve stimulation with 10 ms visible 532 nm laser pulses. (e) Analysis of nerve stimulation with 10 ms near-infrared (NIR) laser pulses. (f) Analysis of nerve stimulation with different NIR laser pulse lengths. Functional analysis of nerve stimulation in (d-f) is achieved through analysis of the electromyography (EMG) signal (left), maximum amplitude of the compound action potential (CAP, center), and maximum leg displacement distance (right) for the same 12 subsequent stimulation events at a frequency of 1 Hz. EMG signals at selected conditions are presented as an average; shaded area encompasses±SD. For CAP amplitude and maximum leg displacement, the boxes bind IQR divided by the median, and whiskers extend 1.5±IQR. All datapoints are plotted, unless not detected (N.D.). Data in panels (d) and (e) were recorded in the same experimental setup with only a change of the applied laser, and data in panel (f) was acquired in a separate experiment. All results are representative of at least four independent experiments using different membranes and rats. (g) Schematic diagram of a laser scanning and selective nerve activation. (h-i) Selectivity index for a muscle stimulation with different laser positions and power. GM—gastrocnemius medialis, TA—tibialis anterior, PL—plantar interossei.

FIG. 7: Scanning electron microscopy analysis of the wafer surface after etching with 1% nitric acid for a specified amount of time. High magnification images show that the sponge-like morphology of the pore surface is not affected by the etching time. Results are representative of over three independent experiments.

FIG. 8: Scanning electron microscopy analysis of the wafer surface after etching with 1% nitric acid for a specified amount of time. Low magnification images show growth and eventual coalescence of the microscale pillar-like structures. Results are representative of over three independent experiments.

FIG. 9: Cross sectional scanning electron microscopy images of stain etched silicon substrates using 1% nitric acid. (a) High magnification images show formation of porous silicon layer in which its thickness does not increase with the etching time. (b) Low magnification images show growth of microscale pillar-like structures with the increased etching time. hₚ—approximate height of the pillar-like structures. Results are representative of over three independent experiments.

FIG. 10: Nanoindentation measurements. Load-unload curves for (a) nanoporous Si, and (b) plasma-treated nanoporous Si. (c) Summary of calculated mechanical properties. For each group, 3 positions were measured on 2 samples for the total of 6 measurements.

FIG. 11: Spectroscopic ellipsometry measurement of porous silicon sample. Polarization state components Iₛ and I_{c} were recorded using different spot sizes in the wavelength range of 1.5-2.0 eV. Iₛ and I_{c} are parameters directly measured by the spectroscopic ellipsometer that are often transformed into 4W and A components for fitting into models of materials optical constants. Both parameters are sensitive to the local changes in the optical constants and recorded parameters are representative of the material volume irradiated by the selected spot size. The small difference between values measured using large and small spot sizes suggests good uniformity of the porous layer on the millimeter scale in the entire volume.

FIG. 12: Energy dispersive X-ray spectra of the silicon wafers stain etched with 1% nitric acid before (a) and after (b) oxygen plasma treatment. Elemental analysis reveals increase in the surface oxygen content after the treatment.

FIG. 13: Fluorescence spectrum of silicon wafers stain etched with 1% nitric acid for 1 min before and after oxygen plasma treatment. Oxygen plasma reduces radiative recombination and effectively quenches fluorescence in the material.

FIG. 14: Time-dependent etching of silicon membranes in 1% HNO₃ in HF. The optimal photocurrents are reached right before the membrane breakdown. The optimal etching time might depend on the batch of the reagents, but for the same combination of acids and SOIs it stays consistent between experiments.

FIG. 15: Photocurrents obtained using stain etching with different concentrations of nitric acid for 10 s. Concentration higher than 1% significantly reduce photocurrents. The boxes bound interquartile range (IQR) divided by the median, and whiskers extend 1.5×IQR. Each box is calculated from at least three different locations on four separate samples for Nₘₑₐₛ≥12. All datapoints are plotted.

FIG. 16: Comparison of photocurrents recorded from stain etching performed using 1% nitric acid, 1 M iron (Ill) cations, and 0.1 M vanadium (V) oxide as hole-injecting oxidants. Improvement in the photocurrent generation was observed for all oxidants, but only nitric acid generated photocurrents independent of the etching time (self-limiting), and achieved photocurrents were in general higher than for other oxidants. The data for nitric acid is reused from FIGS. 1 and S1, and shown here for direct comparison with other oxidants. The boxes bound interquartile range (IQR) divided by the median, and whiskers extend 1.5×IQR. Each box is calculated from at least three different locations on four separate samples for Nₘₑₐₛ≥12. All datapoints are plotted.

FIG. 17: Scanning electron microscopy images of wafers' surface after stain etching using different oxidants. Only nitric acid showed a sponge-like morphology. Concentrations: 1% nitric acid, 1 M Fe³⁺, 0.1 M V₂O₅. Scale bar, 100 nm. Four samples for each condition were prepared for photocurrent measurements and one of each was imaged.

FIG. 18: Photocurrents obtained after stain etching different types of wafers with 1% nitric acid for 1 min. Photocurrents and their significant enhancement after oxygen plasma treatment was observed only for a p-type wafer. The boxes bound interquartile range (IQR) divided by the median, and whiskers extend 1.5×IQR. Each box is calculated from at least three different locations on four separate samples for Nₘₑₐₛ≥12. All datapoints are plotted.

FIG. 19: Proposed schematic band diagrams of the non-porous/porous silicon interfaces in materials obtained through self-limiting stain etching in p-type (a), intrinsic (b) and n-type (c) silicon substrates. Only the p-type silicon substrate yields the desired light-induced charge separation.

FIG. 20: Photocurrents obtained using stain etching with 1% nitric acid for 1 min and addition of 0.1% (w/v) surfactants and the scanning electron microscopy images of the obtained porous materials. (a) A selection of ionic and non-ionic surfactants were tested to investigate the effect of hydrogen bubble formation on the surface microstructure. In general, addition of surfactants was detrimental to the recorded photocurrents and pure formulation yielded the highest currents. The boxes bound interquartile range (IQR) divided by the median, and whiskers extend 1.5×IQR. Each box is calculated from at least three different locations on four separate samples for Nₘₑₐₛ≥12. All datapoints are plotted. (b) While surfactants affect the microstructure formation and pore morphology, there does not seem to exist a direct correlation between the appearance of material surface and recorded photocurrents. As observed above, the addition of sodium dodecyl sulfate (SDS) eliminated the micropillars. However, the observed photocurrents were higher than when Pluronic P123 was used, in which the micropillars were preserved. The results suggest that pore morphology is a primary factor determining the photoelectrochemical performance of the materials. Four samples for each condition were prepared for photocurrent measurement and one of each was imaged.

FIG. 21: Electrochemical investigation of photoelectrochemical reactions at the nanoporous silicon interface. (a) Cyclic voltammetry scans of porous silicon electrode in different pH in the dark. (b) Cyclic voltammetry scans of porous silicon electrode in different pH under illumination. As the standard potential of oxygen reduction reaction (that is related to the proton concentration, amount of dissolved oxygen, etc.) is dependent on pH, the overpotential to activate the reaction will also change hence leading to a shift in the peak position. (c) Dye degradation assays shows production of hydrogen peroxide under illumination at the interface with porous silicon, which is further increased upon light illumination. (NC—negative control, no material present) The boxes bound interquartile range (IQR) divided by the median, and whiskers extend 1.5×IQR. Each box is calculated from six technical replicates for Nₘₑₐₛ=6. All datapoints are plotted.

FIG. 22: Electrochemical impedance equivalent circuit. (a) Equivalent circuit used for modelling impedance spectra. R₁ is the contact resistance, R₂ is the charge transfer resistance, Q is constant phase element (with parameter α), R_{d} is restricted diffusion (with parameter t_{d}). (b) Fitting results for porous silicon in the dark and under 532 nm LED illumination. (c) Parameters obtained from model fitting.

FIG. 23: Analysis of the stability of photocurrents in the silicon membrane. (a) Short-term stability shows that capacitive currents decrease during the first hour, but faradaic currents can significantly increase leading to the increase in total injected charge. No significant difference in the trends was observed whether the membrane was stored in the dark or under repeating 4 Hz illumination suggesting photostability of the observed photoelectric effects. Each datapoint represents mean of 3 measurements on the single membrane. Lines do not represent real data and are used to visualize the trend. (b) Long-term static stability analysis shows that total injected charge decreases approximately by 50% during the first 24 hours in 1×PBS at 37° C., and to the insignificant values over the 3 days for the regular nanoporous Si samples (Ctrl). Deposition of titianium/aluminum oxide dielectric layers through atomic layer deposition (ALD) increased material's stability. The boxes bound interquartile range (IQR) divided by the median, and whiskers extend 1.5×IQR. Each box is calculated from at least three different locations on four separate samples for Nₘₑₐₛ≥12. All datapoints are plotted. (c) ALD allowed to preserve 60% of photocurrent intensity of the time of 48 hours. Mean normalized photocurrents are shown.

FIG. 24: Analysis of the power- and wavelength-dependent photoresponse in a silicon wafer stain etched with 1% nitric acid for 1 min. (a) 532 nm and 625 nm light generated comparable photocurrents. Significantly less photocurrents were observed for 808 nm light. (b) No significant photocurrents were recorded with 365 nm light, and low-level compound signal of capacitive and thermal photocurrents was observed. The dataset is a series of measurements for single sample.

FIG. 25: Microspectrometry of silicon membranes. Transmittance and reflectance spectra of as etched Si membrane (a) and Si membrane after stain etching and plasma treatment (b). The white area on the plot represents absorbance. (c) Comparison of membranes absorptance before and after processing.

FIG. 26: Physiology measurements of the isolated heart paced at 6 Hz with 1.7 ms 532 nm laser pulses. Electrocardiography (ECG) recording shows efficient pacing during the period of the light pulsing. The heart contractions adjust mechanically to the new pacing conditions which is indicated by the increasing left ventricular pressure (LVP) signal. The stimulation using these conditions can be maintained for more than 1 hour in the isolated heart setting.

FIG. 27: Pacing of the isolated heart using dual chamber stimulation and different spacing between light pulses. At describes the time difference between LV and RV stimulation. Negative values are used when RV pulse precedes LV pulse. When RV pulse is leading two compound action potentials and two contractions were observed up to the 200 ms spacing between stimulation pulses, but when the order of pulses is reversed, the spacing between pulses must be at least 400 ms apart to observe two contractions.

FIG. 28: The measurement setup and multichannel EMG recordings. (a) Top view of the rectangular silicon membrane wrapped around the sciatic nerve. (b) Overview of the measurement and laser irradiation setup. (c) Plot of 12 overlapping EMG signals recorded in gastrocnemius medialis (GM), plantar interossei (PL) and tibialis anterior (TA) muscles for each laser position using 38 mW/mm² laser power. (d) Plot of 12 overlapping EMG signals for each muscle and laser position using 28 mW/mm² laser power. For the measurement at position 0.5 mm, the sixth signal was excluded from analysis due to surge in an amplifier circuit and only 11 curves are shown.

FIG. 29: Proof of concept implantation of the silicon membrane and fiber cannula in the rat limb and electromyography (EMG) recording upon light stimulation. Overview of the implantation procedure: (a) Membrane wrapped around a sciatic nerve. (b) Nerve repositioned between the muscles. (c) Fiber positioned over the membrane. (d) Muscle closed using Nylon sutures. (e) Multichannel EMG recordings of the compound action potential in the muscle during stimulation (532 nm laser, 20 mW/mm²).

FIG. 30: Macroscopic optical photographs of stain etched silicon. (a) Stain etched silicon wafer showing black-colored porous silicon. (b) Stain etched silicon membrane showing opalescent stains due to thin-film interference. Both samples were etched for 10 s in 1% HNO₃ in HF (v/v).

FIG. 31: Schematics of the designs of the positive photomasks used for fabrication of silicon membranes. (a) Partially stretchable 9-pad design. View of the entire mask (left) and the detail of the meandering interconnect (right). Shapes were loaded for direct writing in XOR mode. Units are in millimeters. Continues area (˜10 mm²) designs: (b) hexagon design and (c) rectangular design. Hexagonal array of holes was used in designs (b) and (c) to ease etching and lift-off of large flat surface area membranes.

FIG. 32: Analysis of the photothermal effects. (a) Holding current dependent photocurrents in a wafer stain etched with 1% nitric acid for 20 min and after oxygen plasma treatment. (b) Holding current dependent photocurrents in a silicon membrane stain etched with 1% nitric acid for 10 s and after oxygen plasma treatment. Linear regression showing little correlation between the holding current and recorded photocurrents with negligible slope suggests lack of photothermal currents in the measurements. Dashed line is a fitted linear function and R² denotes the coefficient of determination calculated using residual sum of squares.

FIG. 33: Validation of the photocurrent data analysis framework. Data for silicon wafers etched with 1% nitric acid for different amounts of time is used. (a) Clustering of datapoints for pre- and post-oxygen plasma measurements demonstrate significant effect of this treatment on the photocurrents. (b) Plot of capacitive vs. faradaic vs. injected charge, expected linear relationship. (c) Plots shows no apparent clustering in capacitive and faradaic currents depending on the order of measurements. (d-f) Plots show no relationship between pipettes resistance and recorded photocurrents, e.g., some range of resistance recording only high or only low values. Clustering around measurement number is expected, because samples in the same series were measured together until finished or pipette was completely broken. Values recorded with <1 MΩ correspond to partially damaged pipettes. Pipettes were not pulled to this resistance.

FIG. 34: Additional information on the photoelectrochemistry measurements. (a) Schematic of the silicon electrode assembly and (b) photograph of representative electrodes. (c) Schematic of the photoelectrochemical cell (WE—working electrode, CE—counter electrode, REF—reference electrode), and (d) photograph of the experimental setup.

FIG. 35: Scanning electron microscopy images of a sample lamella used for scanning transmission electron microscopy imaging. (a) Low magnification image showing sample on the copper half-circle grid. (b) Only part of the lamella was thinned to <200 nm. (c) Thinned region with visible porous and non-porous material domains. (d) Backscattered electron image showing platinum and gallium deposits on the lamella.

FIG. 36: Control experiments for the photoelectrochemical heart pacing. (a) Membranes or laser pulses alone have no effect on the heart contractions. Successful stimulation was observed only when both devices and light pulses were present. Similarly, unetched Si membranes (b) and gold-decorated p-i-n membranes (c) were not able to stimulate the heart.

FIG. 37: Spectra of light sources used in this study. (a) 365 nm LED. (b) 532 nm LED. (c) 625 nm LED. (d) 532 nm laser. (e) 808 nm laser.

FIG. 38: Schematic of the optical setup used for microspectrometry of silicon membranes.

### DETAILED DESCRIPTION

Before the disclosed methods and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

Homo- or heterojunctions play essential roles in semiconductor-based devices such as field-effect transistors, solar cells, photodetectors, and light-emitting diodes. Semiconductor junctions have been recently used to optically trigger biological modulation via photovoltaic or photoelectrochemical mechanisms. The creation of heterojunctions typically involves materials with different doping or composition, which leads to high cost, complex fabrications, and potential side effects at biointerfaces. Here it is shown that a porosity-based heterojunction, a largely overlooked system in materials science, can yield an efficient photoelectrochemical response from the semiconductor surface. Using self-limiting stain etching, a nanoporous/non-porous, soft-hard heterojunction was created in p-type silicon within seconds under ambient conditions. Upon surface oxidation, the heterojunction yields a strong photoelectrochemical response in saline. Without any interconnects or metal modifications, the heterojunction enables efficient non-genetic optoelectronic stimulation of isolated rat hearts ex vivo and sciatic nerves in vivo with optical power comparable to optogenetics, and with near-IR capabilities.

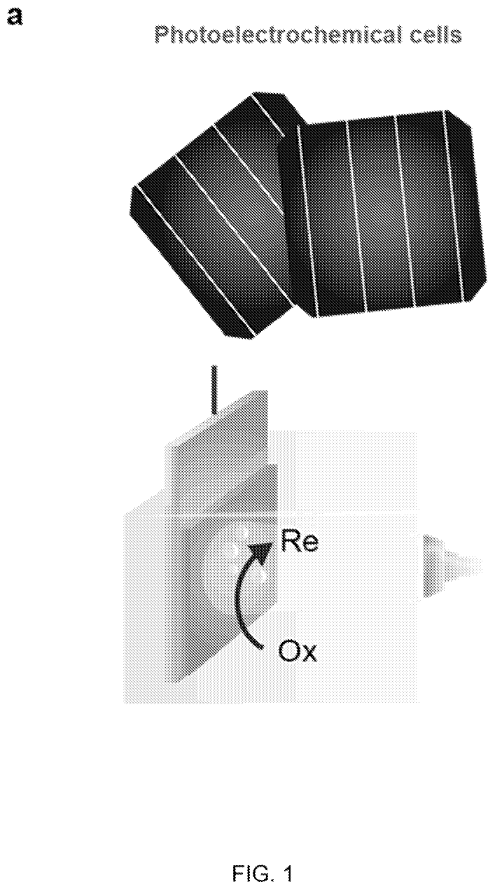

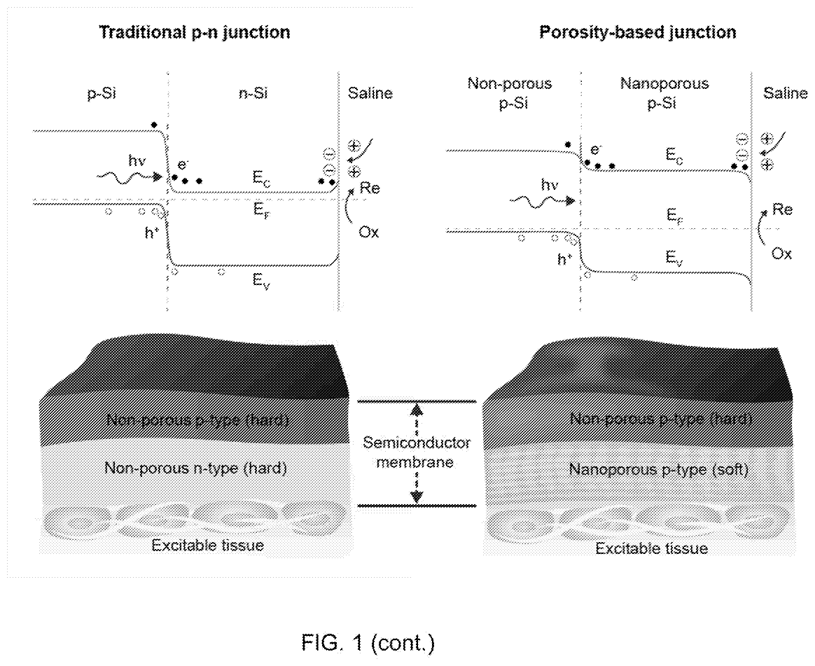

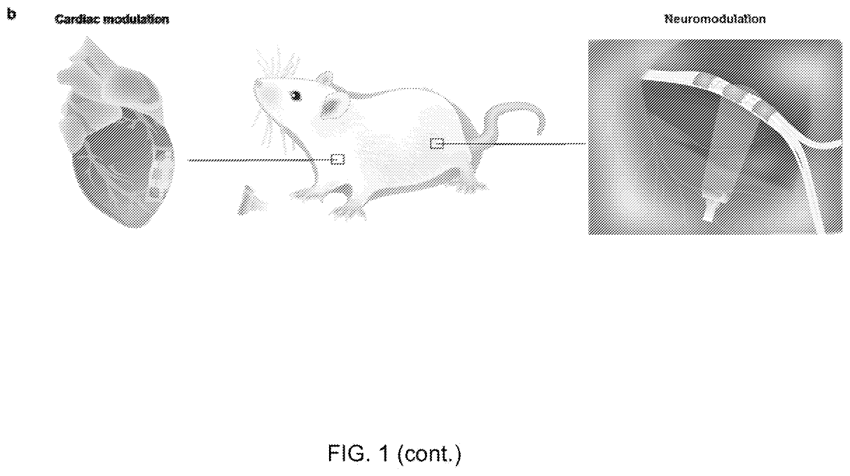

An alternative, yet much less explored, strategy for diode-like performance involves creating a nanoporous/non-porous semiconductor heterojunction²⁴⁻²⁶, wherein the nanoporous and non-porous semiconductor components display different band structures (FIG. 1a, upper panel). Such a porosity-based semiconductor heterojunction has not been used for bioelectronics studies. However, it represents an appealing candidate as it yields a pure semiconductor-biofluids interface (i.e., dopant- or metal-free) and a potentially more deformable biointerface due to the porosity of the nanoporous layer. Interestingly, it is noted that the nanoporous/non-porous semiconductor heterojunction, upon establishing interfaces with tissues at the nanoporous surface, is reminiscent of the endocuticle/exocuticle heterostructure in the chitin-based exoskeleton where the softer endocuticle layer attaches directly to the epidermis layer and helps mediate the soft-hard biointerface (FIG. 1a, lower panel).

In the present disclosure, a fast and efficient method for obtaining pure-silicon porosity-based heterojunctions with strong photoelectrochemical properties is developed by combining stain etching and high-power oxygen plasma treatment. To demonstrate the utility of such materials in optically induced biomodulation, ex vivo heart pacing and in vivo sciatic nerve stimulation were performed (FIG. 1b). Flexible crystalline silicon membranes were fabricated that transduce light pulses with low optical densities to allow overdrive heart pacing and nerve bundle activation leading to skeletal muscle contraction. It is believed that this new class of pure-silicon optical biological modulator may have therapeutic applications, such as cardiac pacing and peripheral nerve regeneration.

Homo- or heterojunctions play essential roles in semiconductor-based devices such as field-effect transistors, solar cells, photodetectors, and light-emitting diodes. Semiconductor junctions have been recently used to optically trigger biological modulation via photovoltaic or photoelectrochemical mechanisms. Creation of heterojunctions typically involves materials with different doping or composition, which leads to high cost, complex fabrications, and potential side effects at biointerfaces. Here it is shown that a porosity-based heterojunction, a largely overlooked system in materials science, can yield an efficient photoelectrochemical response from the semiconductor surface. Using self-limiting stain etching, a nanoporous/non-porous, soft-hard heterojunction in p-type silicon is created within seconds to minutes. Upon surface oxidation, the heterojunction yields a strong photoresponse in saline. Without any interconnects or metal modifications, the heterojunction enables efficient non-genetic pulse stimulation of isolated rat hearts ex vivo and sciatic nerves in vivo with radiant exposure similar to that used in optogenetics.

As described herein, various embodiments of the disclosure involve contacting a cell or tissue with the device described herein. With regard to the biological medium contacted, it may be a cell, collection of cells, tissue, or entire organ, all of which may be referred to as forming a device cell interface. This interface allows for photoelectrochemical stimulation of the cell using light through illumination of the associated device.

Accordingly, in one aspect, the present disclosure provides for a device comprising a p-type semiconductor material comprising a nanoporous semiconductor layer and a nonporous semiconductor layer that form a heterojunction; and a flexible substrate comprising one or more of polymers on which the p-type semiconductor material is distributed such that the flexible substrate is in contact with the nonporous semiconductor layer. In some embodiments, the p-type semiconductor material is pre-treated. For example, in certain embodiments as otherwise described herein the p-type semiconductor material is oxygen (O₂) plasma-treated p-type semiconductor material. For example, the present disclosure provides for a device comprising a p-type silicon material comprising a nanoporous silicon layer and a nonporous silicon layer that form a heterojunction; and a flexible substrate comprising one or more of polymers on which the p-type silicon material is distributed such that the flexible substrate is in contact with the nonporous silicon layer.

Many suitable flexible substrates are known in the art. In certain embodiments as otherwise described herein, the flexible substrate comprises one or more polymers, and the one or more polymers is selected from a biocompatible polymer, a biodegradable polymer, an extracellular matrix protein, and a combination thereof. For example, in some embodiments, the polymer is polydimethylsiloxane, poly(methyl methacrylate), poly lactic-co-glycolic acid, poly(ethylene glycol) diacrylate, collagen, or gelatin (e.g., is polymethylsiloxane). In particular embodiments, the flexible substrate comprises polydimethylsiloxane, or is a polydimethylsiloxane substrate.

In some embodiments, the flexible substrate may be porous. In certain embodiments, the flexible substrate has an open porosity of at least about 10%. For example, the flexible substrate has an open porosity of at least about 20%, or 30%, or 40%, or 45%, or 50%, or 55%, or even 60%. In various embodiments, the flexible substrate has an open porosity of no more than 80%, or no more than 75%. In some embodiments as otherwise described herein, the nanoporous semiconductor layer comprises pores having cavities and/or channels. In other embodiments, the flexible substrate is non-porous.

The nanoporous semiconductor layer can be prepared from any suitable semiconductor material, including purified silicon. Silicon, such as silicon wafer or silicon-on-insulator wafer, is widely available commercially, for example from Nova Electronic Materials (Flower Mound, TX, USA). In various embodiments, the silicon is p-type silicon. As described herein and known in the art, nanoporous semiconductor may be prepared according to various methods. For example, nanoporous silicon may be prepared through contacting a silicon wafer with the etching solution that contains both the hydrofluoric acid and an oxidant. In particular embodiments as otherwise described herein, the nanoporous silicon is prepared through an etching step, wherein the etching comprising contacting silicon with hydrofluoric acid and an oxidant. The oxidant can be nitric acid, or hydrogen peroxide, or an Fe(Ill) salt (e.g., Fe(Ill) chloride hexahydrate), or vanadium(V) oxide. In various embodiments, the etching is performed with a mixture of nitric acid (i.e., the oxidant) and hydrofluoric acid. In such embodiments, the etching may be metal-free etching. The etching time may be adjusted according to reactant concentrations and desired device characteristics. In various embodiments, the nanoporous silicon may be defined as that subjected to at least some amount of etching.

For planar silicon wafers with exposed silicon on both surfaces, etching is advantageously performed on only one side in order to create a single heterojunction. Accordingly, one side of the silicon wafer may be passivated with a polymer or inorganic material as known in the art. Additionally or alternatively, the etching solution may be applied to only one side of the wafer, for example, by drop casting the etching solution onto the silicon wafer.

Critically, etching can be performed so that the semiconductor wafer is only partially etched, e.g., only one face of the semiconductor wafer is rendered nanoporous. In such cases, there can advantageously exist a heterojunction between the etched, nanoporous semiconductor and unetched, nonporous semiconductor. As described herein, the presence of such a junction provides various desirable electronic and optoelectronic properties. After etching, the semiconductor layer may be subjected to additional process steps, such as optional cleaning and/or plasma treatment.

It has been surprisingly determined that nanoporous semiconductor layers afford enhanced photocurrents which can be effectively used in various modes of biomodulation. Accordingly, in certain embodiments as otherwise describe herein, the nanoporous semiconductor layer is mesoporous, wherein the nanoporous semiconductor layer has a pore size in the range of 2 nm to 50 nm, for example in the range of 1 nm to 25 nm, usually 2 to 10 nm.

The nanoporous semiconductor layer as otherwise described herein can be provided in a variety of suitable thicknesses according to the device characteristics. In certain embodiments as otherwise described herein, the nanoporous semiconductor layer has an average thickness in a range of about 500 nm to 3 μm, for example in the range of 700 nm to 1.5 μm, or in the range of 800 nm to 1.2 μm, or in a thickness of about 1 μm. In some cases, the thickness of the nanoporous semiconductor layer may be adjusted through etching conditions, while other conditions are self-limiting.

As otherwise described herein, the nonporous semiconductor layer is the remaining unetched portion of the semiconductor layer. In various embodiments, the nonporous semiconductor layer is not substantially affected by the etching step, and thus has a porosity and/or electronic structure similar to the original semiconductor wafer. For example, in various embodiments as otherwise described herein, the nonporous semiconductor layer has a thickness in the range of 200 nm to 500 μm, for example 1 to 5 μm.

After the formation of the nanoporous and nonporous semiconductor, the semiconductor wafer may be joined with the flexible substrate. For example, in the semiconductor wafer way be pressed onto a pre-formed substrate, or the substrate may be cast onto the semiconductor wafer. In particular embodiments, PDMS substrate is cast on the semiconductor wafer. Other methods of substrate formation will be apparent to a person of skill in the art in light of the disclosure herein.

As described here, porosity-based semiconductor heterojunctions have been developed with beneficial optoelectronic properties. Various semiconductor materials may be utilized according to the present disclosure. A number of semiconductors can form porous structures, and so may be used to form porosity-based heterojunctions. For example, in various embodiments as otherwise described herein, the semiconductor is silicon, silicon carbide, gallium nitride, gallium arsenide, indium phosphide, cadmium sulfide, cadmium selenide, or cadmium telluride. For example, in particular embodiments, the semiconductor is silicon.

The present device can be utilized for biomodulation of tissue, for example as described in the Examples below. See also International Patent Application no. PCT/US2020/056106, filed on 2020 Oct. 16 (WO 2021/076981, published Apr. 22, 2021), and incorporated herein by reference in its entirety.

As described herein, the illumination of the device can create a photoelectrochemical effect leading to cell activation. The photoelectrochemical effect describes the transfer of energy from light, to a voltage, to a chemical response by a cell or tissue. In another aspect, the present disclosure provides for a method for modulating activity of a cell, the method comprising: contacting a membrane of the cell with the device as otherwise described herein to form a device-cell membrane interface; and exposing the interface to light under conditions to depolarize the cell membrane thereby increase a threshold for activation of the cell, wherein the cell is capable of being activated by the photoelectrochemical effect. See, for example, representative conditions of exposing an interface to light in International Patent Application no. PCT/US2020/056106, filed on 2020 Oct. 16, and incorporated herein by reference in its entirety

The methods of the disclosure modulate activity of a cell or a tissue, including a natural and/or engineered tissue, capable of being activated by photoelectrochemical effect. Thus, in certain embodiments, any cell capable of being activated by electrochemical or photoelectrochemical stimulation may be used in the methods of the disclosure. In some embodiments, the cell is a cardiomyocyte, a neuron, a skeletal muscle cell, a smooth muscle cell, an endothelial cell, an insulin-secreting beta cell, or a retinal cell. In certain embodiments, the cell is a cardiomyocyte. In certain embodiments, the cell is a neuron.

In certain embodiments, the structure-cell interface is a direct interface between the device and the cell membrane (i.e., there are no intervening structures between the silicon device and the cell membrane). In certain embodiments of the methods of the disclosure, the semiconductor device contacts the membrane without penetrating the membrane. For example, the semiconductor device may rest on the surface of the cell membrane.

Another aspect of the disclosure provides methods of treating a disease in a subject by modulating activation of a cell. In certain embodiments of the disclosure, the method is treating a neuronal disease or a neuromuscular disease. In certain other embodiments of the disclosure, the disease is an ophthalmic disease. In certain other embodiments of the disclosure, the disease is associated with the sciatic nerve, such as pain, spinal cord injuries, osteoporosis, unitary and/or bowel incontinence. In certain other embodiments, the methods of the disclosure treat a cardiovascular disease. For example, in certain aspects of the disclosure, methods of the disclosure optically train myocardium to beat at a target frequency. Thus, one aspect of the disclosure provides a method for optically training myocardium to beat at a target frequency. Thus, in another aspect, the present disclosure provides for a method of treating a disease in a subject by modulating activation of a cell, the method comprising: providing one or more devices as otherwise described herein to the affected cell(s) in the subject; and exposing the one or more devices to the light under conditions sufficient to overcome a threshold for activation of the cell(s) and treat the disease. See, for example, representative conditions for treating disease described in International Patent Application no. PCT/US2020/056106, filed on 2020 Oct. 16, and incorporated herein by reference in its entirety

In order to stimulate the cells (e.g., the myocardium) to pulse at a target frequency and/or to train the myocardium to pulse at such a target frequency independent of continued stimulation, pulses of illumination may be applied to the myocardium. Multiple pulses may be provided during a pacing or training period, e.g., at a frequency corresponding to the target myocardial beat frequency (e.g., between 0.5 and 3 Hz). In order to train the myocardium to continue pulsing at the trained frequency even after the optical pulses end, multiple periods of training pulses may be provided, each period of training pulses being separated from the others by a “break period” during which illumination is not provided. The duration of such a break period may be optimized, e.g., to have a two minute duration. Accordingly, in certain embodiments as otherwise described herein, the light pulse has a frequency ranging from 0.25 Hz to 50 Hz, such as ranging from 1 Hz to 5 Hz, or about 4 Hz.

In another aspect, the present disclosure provides for a method for photoelectrochemically pacing or training myocardium in a subject to beat at a target frequency, the method comprising: contacting the myocardium with one or more devices as otherwise described herein; and operating a light emitter to provide, during a training period of time, a plurality of light pulses to the myocardium at the target frequency. In certain embodiments as otherwise described herein, the device is configured to be placed in contact with cells of the myocardium such that the nanoporous semiconductor layer is in contact with cells of the myocardium.

Advantageously, the present disclosure provides for methods of observing the effects of the light administrating and optionally adjusting the treatment accordingly. In certain embodiments as otherwise described herein, the method for photoelectrochemically training myocardium further comprises detecting a pulse rate of the myocardium during a detection period of time, wherein the detection period of time differs from the training period of time. In various embodiments, the detection period of time is subsequent to the training period of time, and wherein the method further comprises: responsive to the detected pulse rate differing from the target pulse rate by more than a threshold amount, operating the light emitter to provide, during an additional training period of time, an additional plurality of light pulses to the myocardium at the target frequency.

Advantageously, the device as described herein can be used in biological settings. Accordingly, in another aspect, the present disclosure provides for a device as otherwise described herein, wherein the device is in contact with a cell (e.g., a plurality of cell, or a tissue).

In another aspect, the present disclosure provides for a method for photoelectrochemically controlling limb movement in a subject, the method comprising: contacting sciatic nerve with one or more devices as otherwise described herein; and operating a light emitter to provide, during a period of time, a plurality of light pulses to the sciatic nerve at the target frequency. In certain embodiments as otherwise described herein, the device is configured to be placed in contact with sciatic nerve cells such that the nanoporous semiconductor layer is in contact with sciatic nerve cells.

The wavelength and power of light utilized for excitation may be adjusted based on the characteristics of the device and tissue of interest, as well as routine considerations of light source and safety. Accordingly, in certain embodiments as otherwise described herein, the light is provided at an excitation wavelength ranging from 400 to 900 nm. In certain embodiments as otherwise described herein, the light is provided at a power in a range of 0.1 mW/mm² to 20 mW/mm², such as 2 mW/mm² to 10 mW/mm².

In another aspect, the present disclosure provides for a system for treating a disease in a subject by modulating activation of a cell, the system comprising: one or more devices as otherwise described herein; a light emitter configured to provide a light pulse to the device, wherein the one or more devices provide, to the cell they are in contact with, excitatory stimulus in response to receiving the light; and a controller that is operably coupled to the light source, wherein the controller comprises one or more processors, wherein the controller is programmed to perform controller operations including: operating the light source to provide the light pulse to the cell. For example, in certain embodiments, the system as otherwise described herein is for electrochemically training myocardium to beat at a target frequency, or a target frequency range.

In certain embodiments as otherwise described herein, the device is configured to be placed in contact with cells of the myocardium such that the nanoporous semiconductor layer is in contact with cells of the myocardium. In other embodiments, the device is configured to be placed in contact with sciatic nerve cells such that the nanoporous semiconductor layer is in contact with sciatic nerve cells.

In various embodiments, the device as otherwise described herein may be oxygen (O₂) plasma-treated (e.g., at least one component is subject to oxygen plasma treatment).

### Examples

### Materials Synthesis and Structure Characterization.

Stain etching is a type of electroless etching enabled by hole injection from strong oxidants in solution phase into the valence band of semiconductors²⁷. Stain etching can produce different types of porous structures on the surface of silicon, and this porous silicon has been explored as coating layers in photovoltaics, photoluminescent agents in bioimaging, and active electrode components in lithium batteries. Porous silicon has an enlarged bandgap compared with bulk silicon²⁴; however, the photoelectrochemical performance of a nanoporous silicon/non-porous silicon heterojunction has not been leveraged for any bioelectronics applications. The focus of the silicon nanostructuring field shifted in recent years to antireflective coatings²⁸. The field has been engulfed by efforts focused on metal-assisted chemical etching, which have produced many nanostructures from photolithography-defined metal masks²⁹. While metal-assisted etching can be used to obtain a variety of morphologies, the semiconductor-metal junctions introduce additional complexity to the system. Thus, the benefits of metal-free self-limiting etching fabrications should not be overlooked, as the simplicity of process and purity of material can lead to improved biocompatibility and stability.

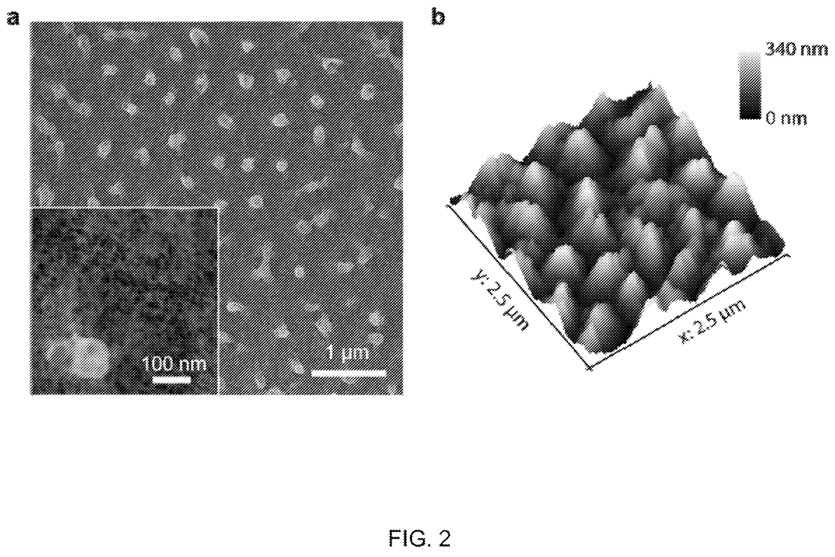

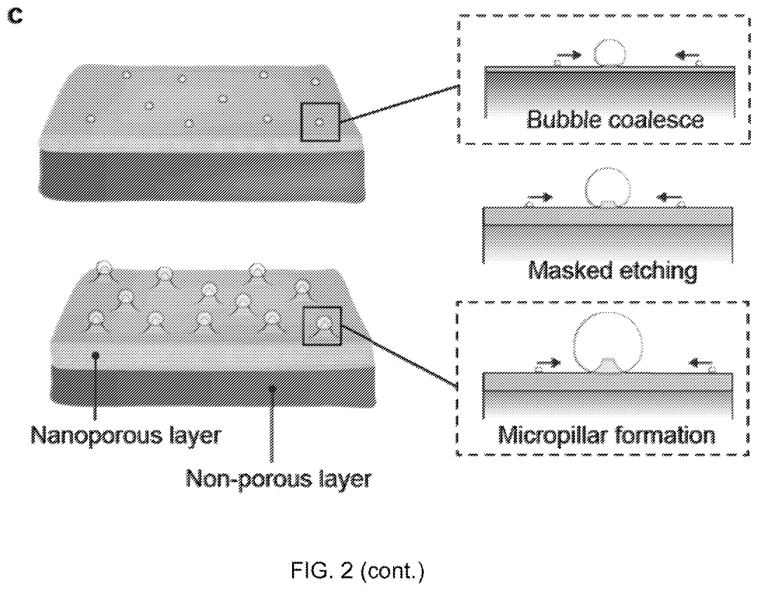

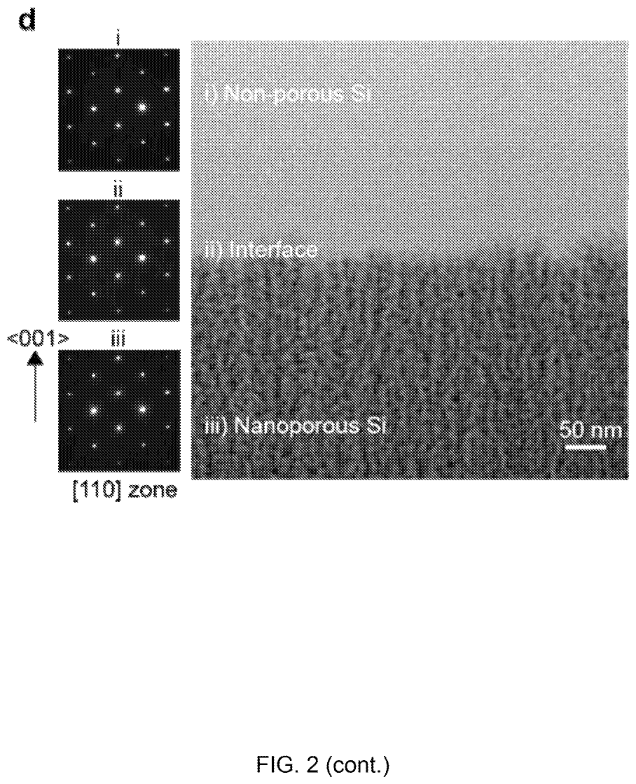

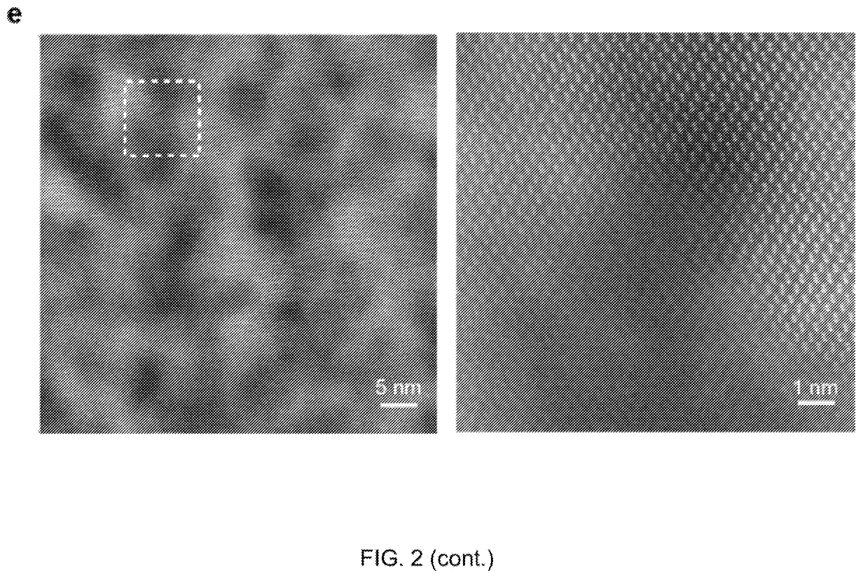

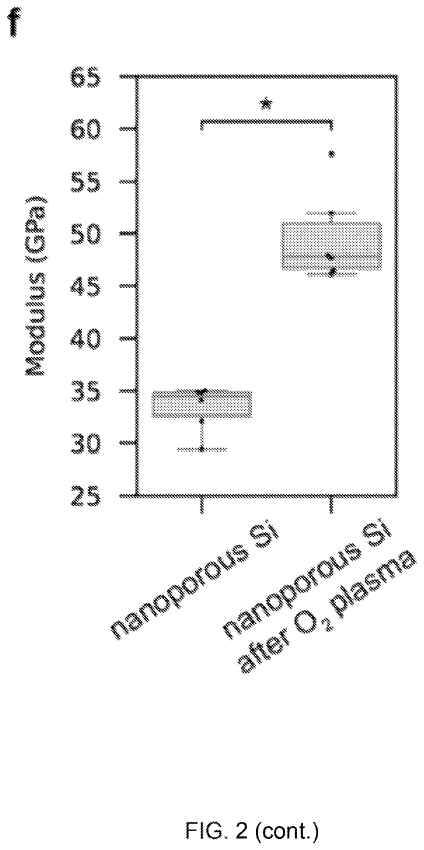

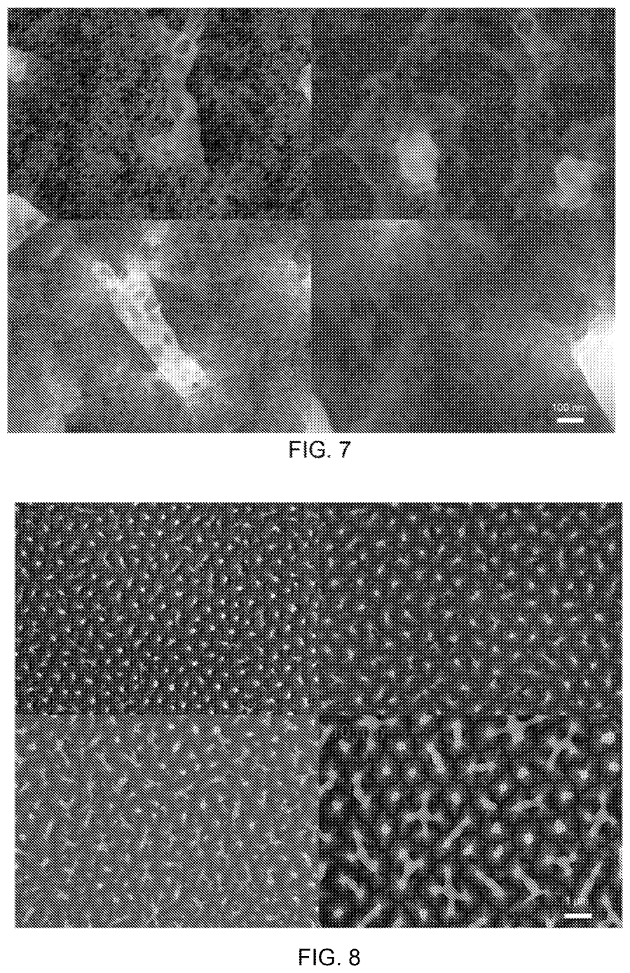

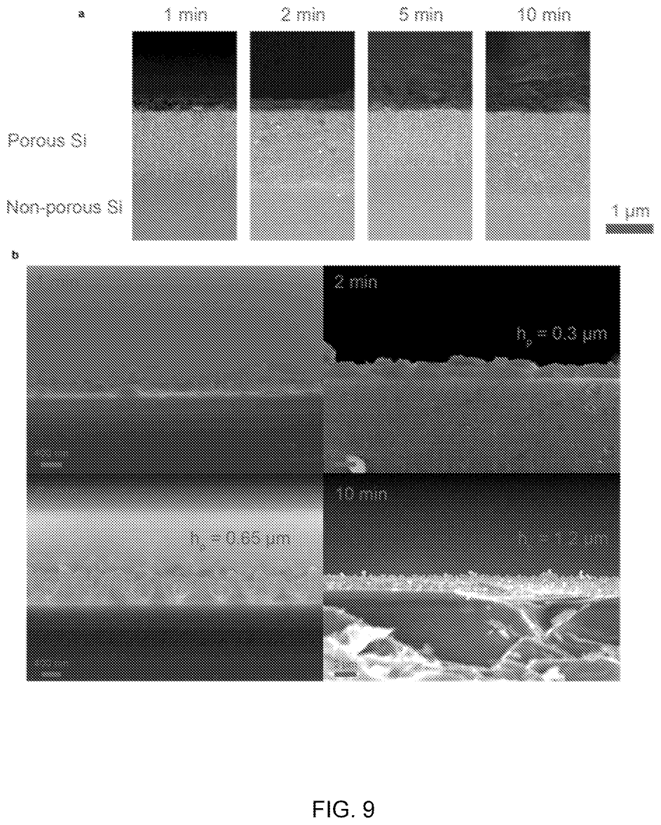

Metal-free nitric acid/hydrofluoric acid-based etching was used to generate nanoporous/non-porous silicon heterojunctions directly in p-type crystalline silicon. Scanning electron microscopy showed formation of porous structures and micro-sized pillars on the surface of the silicon (FIG. 2a, FIG. 7). The sponge-like pore morphology was independent of etching time, but the pillars tended to grow and coalesce over time (FIG. 8). The micropillars formed during etching may contribute to enhanced photoelectrochemical properties through increased surface light trapping²⁸. Pillar height was determined by atomic force microscopy to be in the range of hundreds of nanometres (FIG. 2b). The formation of micropillars is attributed to the coalescence of hydrogen bubbles (self-masking process), which results in heterogenous surface morphology (FIG. 2c). Cross-sectional microscopy showed that while the thickness of the nanoporous layer correlated poorly with etching time, the microstructured pillars grew continuously during etching (FIG. 9).

Scanning transmission electron microscopy (STEM) was used to further understand the microscopic structure of the material. STEM images showed a sharp interface between nanoporous and non-porous domains in a pure silicon sample (FIG. 2d, right). Selected area electron diffraction patterns (FIG. 2d, left) revealed that both domains and their interface were a coherent single crystal. High-magnification STEM images (FIG. 2e) further confirmed the crystalline lattice of the nanoporous silicon domain. A coherent single crystal structure yields efficient charge transport due to the absence of amorphous regimes, while the nano-sized features in the nanoporous domain produce the band shift necessary to enable diode-like behavior (FIG. 1a, upper) through the quantum confinement effect.

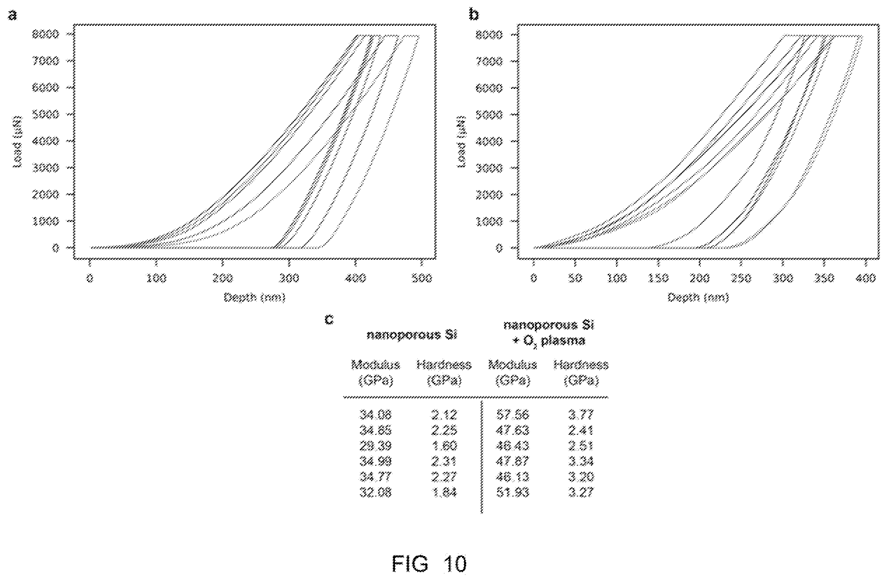

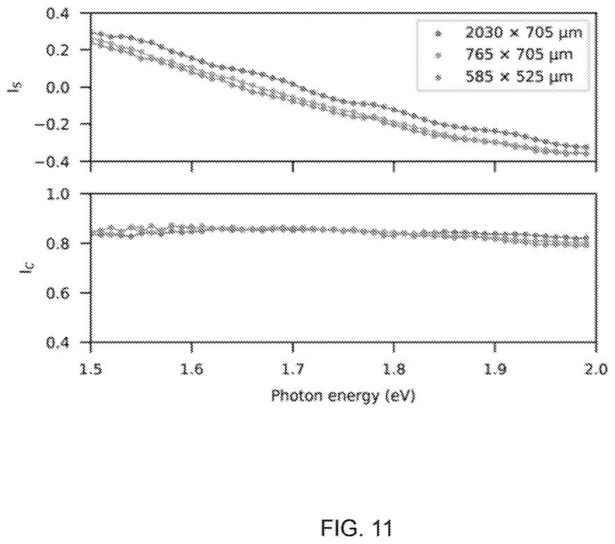

Indentation was used to study the mechanical properties of the nanoporous layer. The elastic modulus of the nanoporous silicon layer was determined to be 33±2 GPa (FIG. 2f, FIG. 10), which is smaller than that of single crystalline silicon³⁴. Oxygen plasma treatment increased the modulus to 50±4 GPa (FIG. 2f), which suggests that oxidation of the nanoporous silicon may have mitigated the mechanical impact of the structural defects in silicon by introducing the atomic- or nanoscale oxide phase. Additionally, spectroscopic ellipsometry was used to determine the uniformity of the porous layer. The high homogeneity of elliptical properties can be found locally on a surface area of sub-millimeter scale. However, when areas of over 1 mm were scanned, larger differences were observed, probably due to changes in the material roughness (FIG. 11). However, such difference does not seem to affect the photoelectrochemical properties of the material which were found to be uniform as described in the following sections.

### Enhancement in Photocurrent Generation

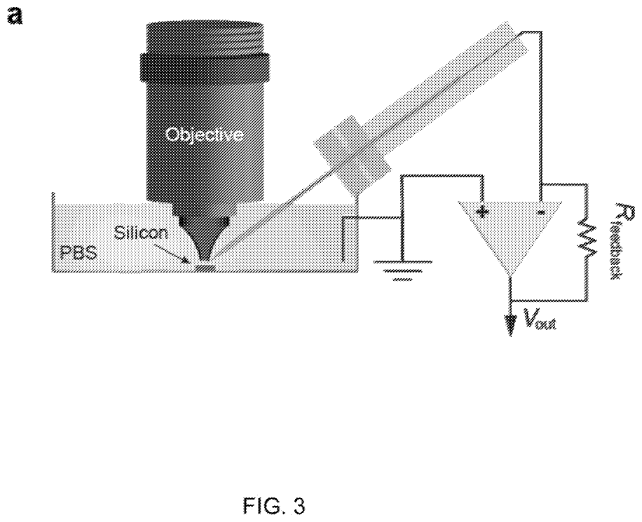

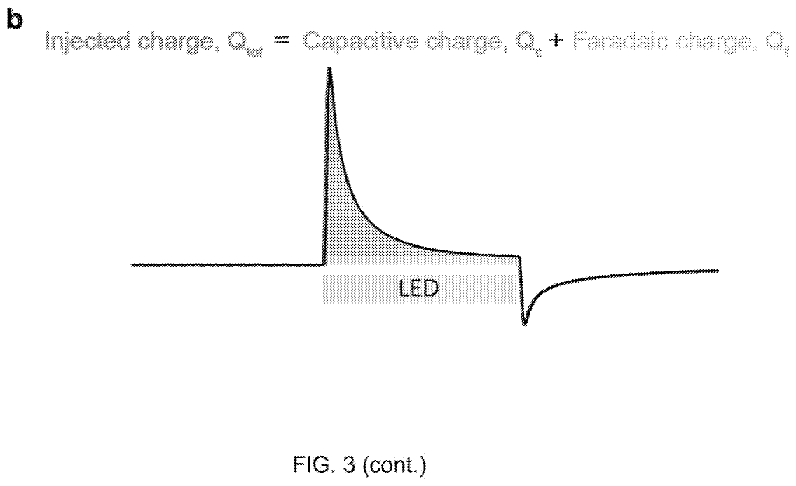

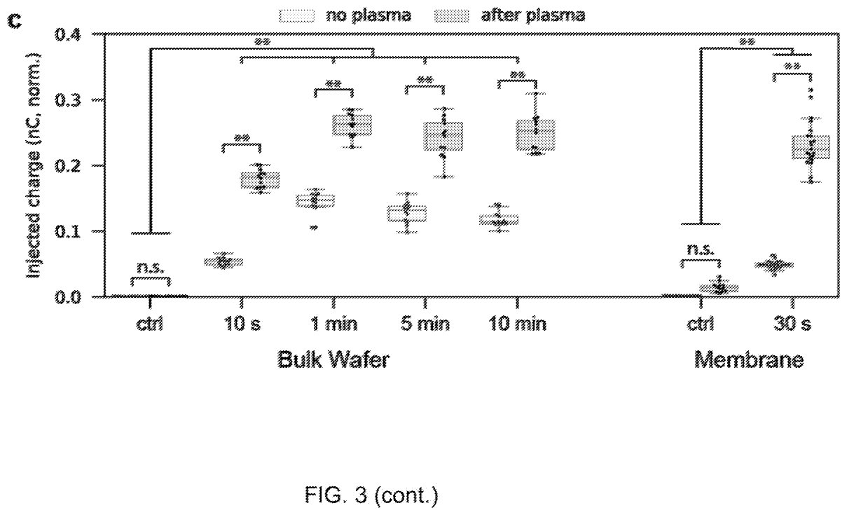

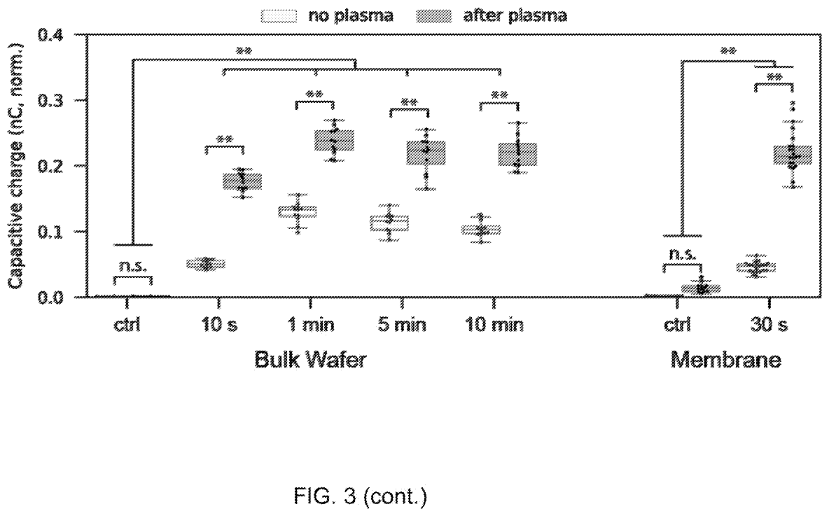

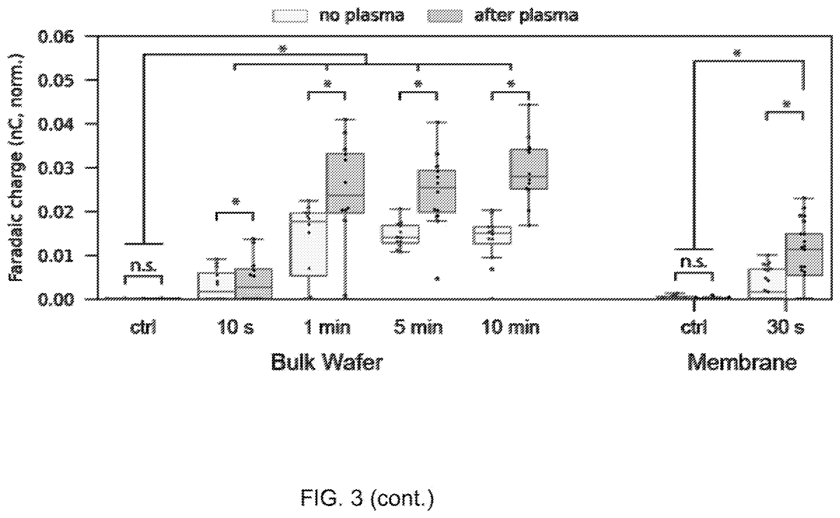

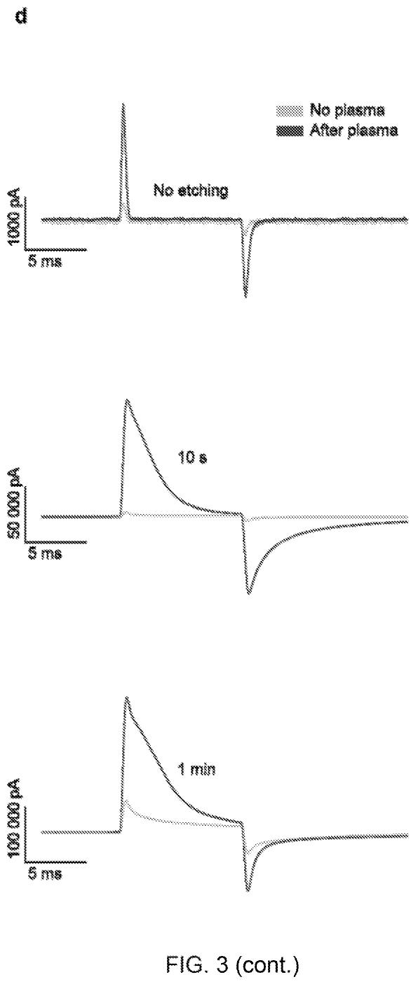

To create a highly efficient photoelectrochemical silicon surface, different stain etching conditions were investigated, along with oxygen plasma treatment. The previously reported patch-clamp photoresponse measurement setup (FIG. 3a) was used to screen a large set of etching conditions on bulk silicon wafers and silicon membranes³⁰. In the analysis, the total charge injection is compared over the illumination period, and the contributions coming from capacitive and faradaic currents (FIG. 3b) determined from the numerical analysis of recorded current transients. For each etching condition, measurements before and after oxygen plasma treatment were performed. FIG. 3c and FIG. 10 shows photocurrents achieved using 1% (v/v) nitric acid in concentrated (50%) hydrofluoric acid. Both high capacitive and faradaic currents were necessary to achieve large overall charge injection. Oxygen plasma treatment enhanced the photocurrents for etching times between 1 and 20 min, with comparable saturation levels. The time-independence of the achieved photocurrents may be due to the self-limiting nature of the stain etching process; the valence band of the porous silicon becomes significantly low such that hole injection and over-etching are inhibited. It was found that even 10 s of this stain etching was sufficient to transform non-photoresponsive p-type silicon wafers into strong photoresponsive materials (FIG. 3d).

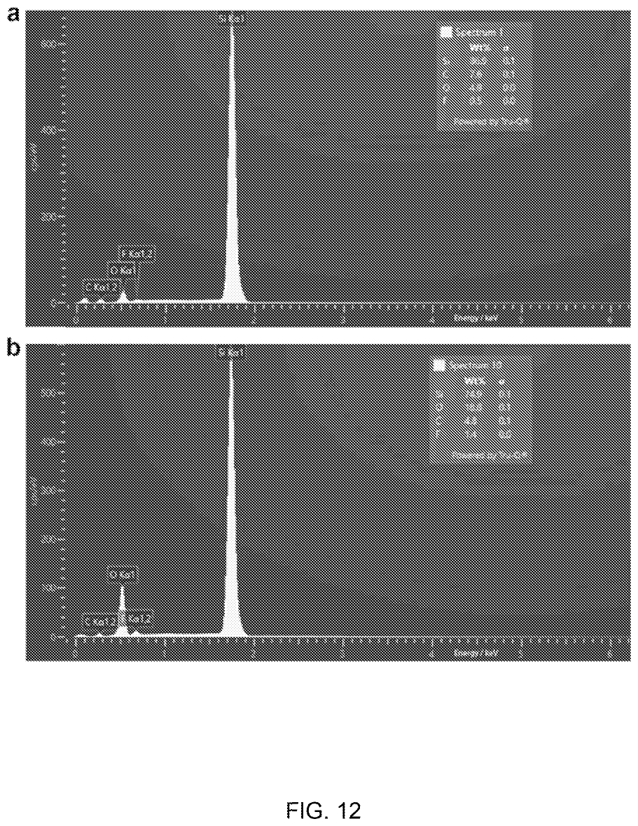

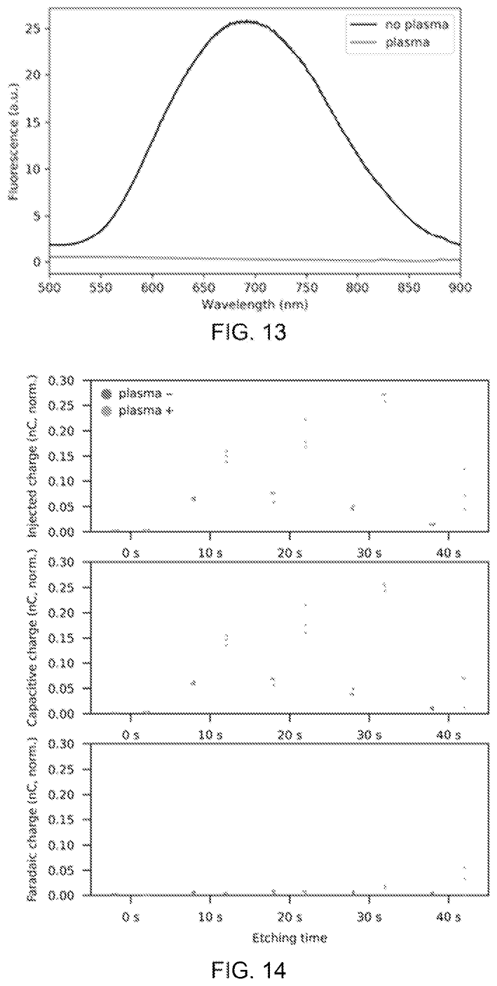

In the absence of the standard p-i-n or p-n doping regime, the stain etching-derived heterojunction was able to generate strong photocurrents upon light illumination. This fast and simple stain etching technique was performed under ambient temperature and pressure, using only wet etching solution, and without the need for any instrumentation. Notably, oxygen plasma treatment that passivated the pore surface with a thin silicon oxide layer enabled a further 4- to 10-fold enhancement of the photocurrents. Electron dispersive X-ray spectroscopy confirmed oxidation of the silicon surface; the oxygen content after plasma treatment increased from 4.9% to 18.8% (FIG. 12). Besides increasing the hydrophilicity of the material such that water and ions could access the nanoscale pores, it is believed that the oxygen plasma may have eliminated radiative surface recombinations³¹, given that the characteristic orange fluorescence was completely quenched upon plasma treatment (FIG. 13).

The fabrication approach is also compatible with the processing of ultrathin, soft, and flexible silicon membranes (thickness ˜2 m) fabricated from silicon-on-insulator (SOI) substrates. For silicon membranes, prolonged etching times over 40 s caused the breakdown of the heterojunction (FIG. 14). But if the supportive crystalline silicon layer was not completely etched (due to the vertical progression of the etching front), the heterojunction generated photocurrents sufficient for biomodulation experiments.

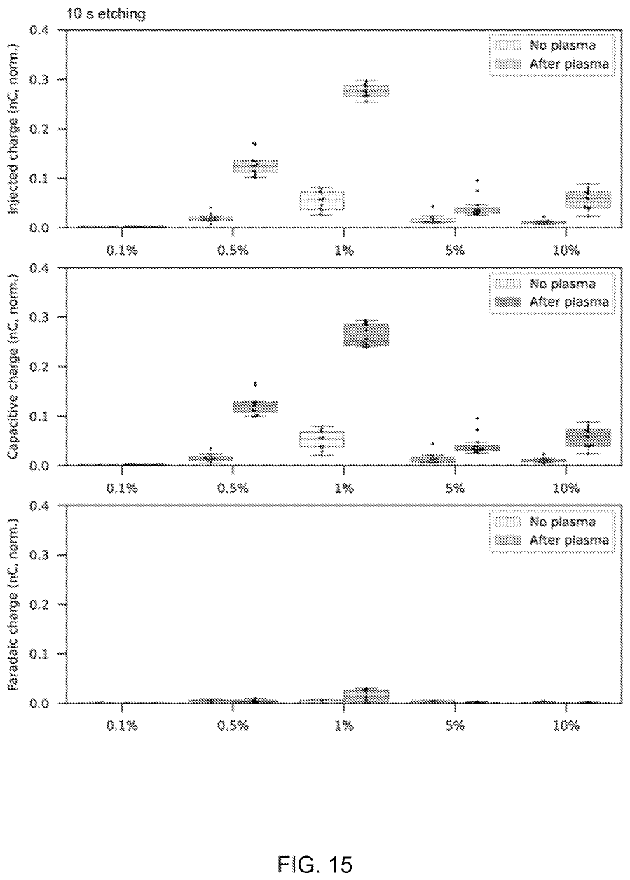

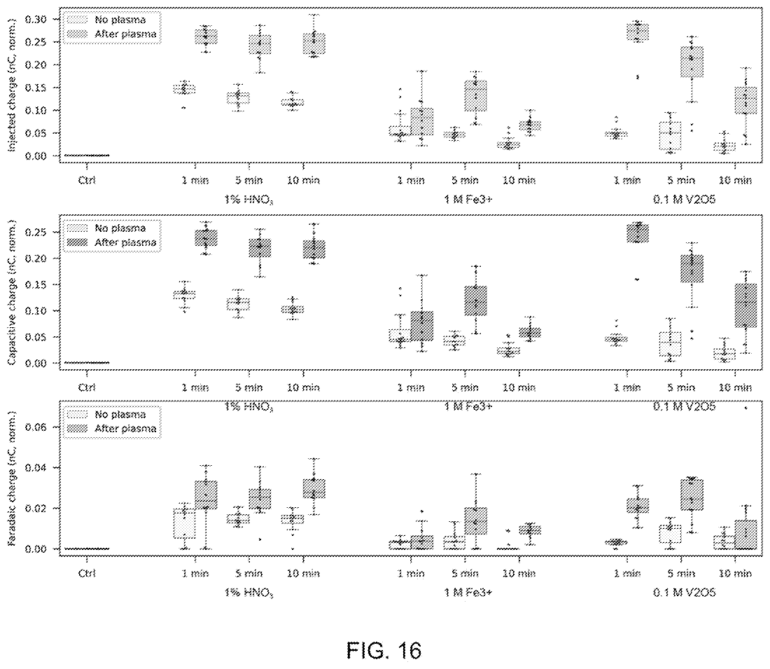

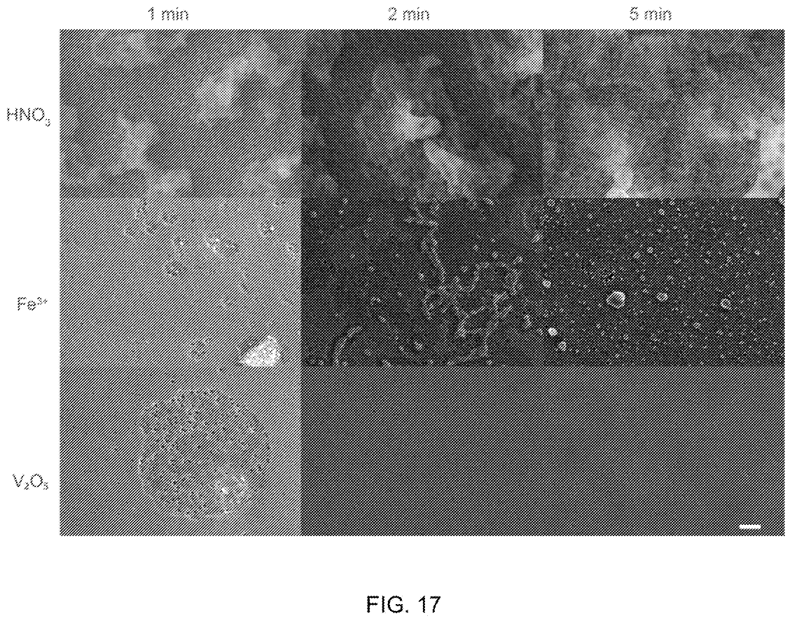

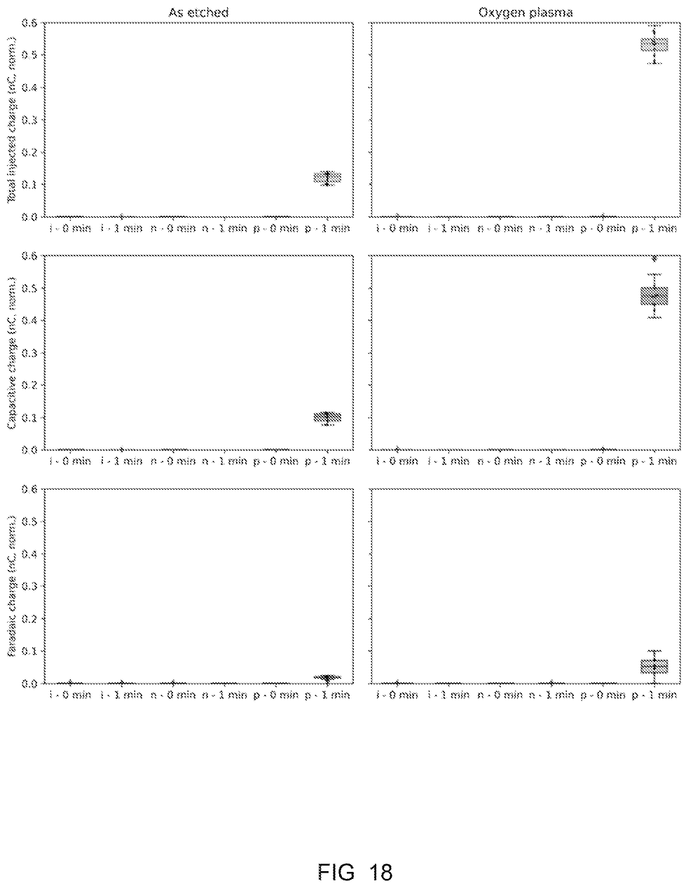

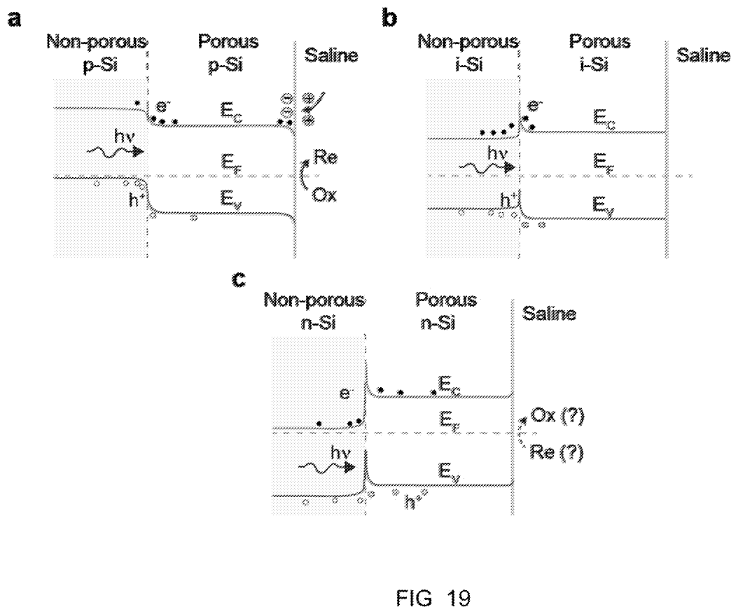

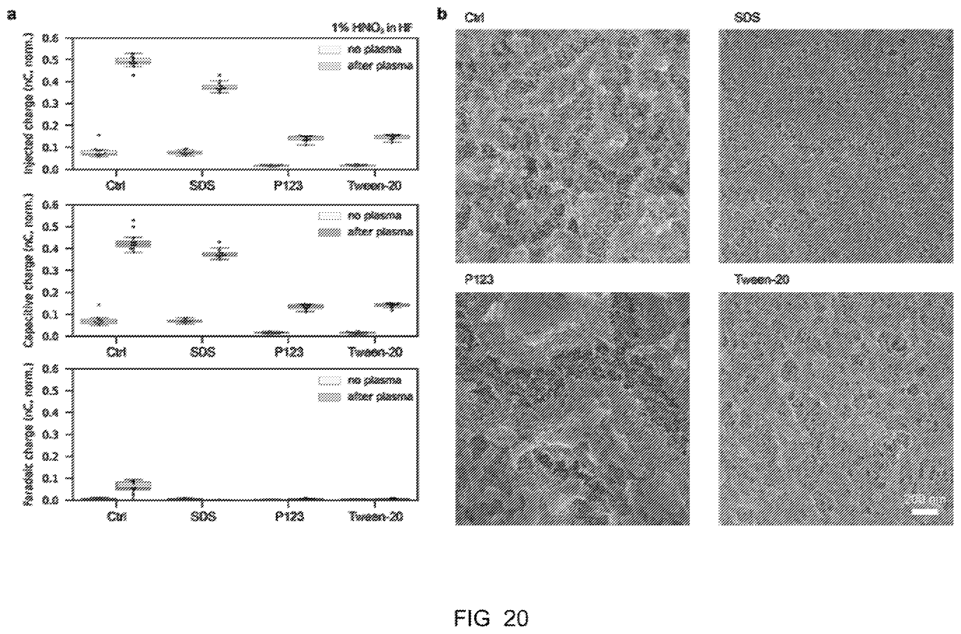

The effect of varying concentrations of nitric acid in the etching solution on heterojunction generation was studied, and it was found that 1% nitric acid concentration was optimal for 10 s stain etching time (FIG. 15). Additionally, ferric ion and vanadium oxide as alternative oxidants for stain etching were investigated, but their performance was lower than that of nitric acid (FIG. 16), possibly due to the suboptimal pore morphology, which had a more solid structure than the spongy surface created by nitric acid (FIG. 17). Notably, it was found that stain etching of n-type and intrinsic silicon wafers did not result in the creation of photoelectrochemical materials (FIG. 18). This is attributed to the dopant-dependent energy levels produced in the silicon substrate upon porosification (FIG. 19). These results support the proposed porosity-based heterojunction mechanism (FIG. 1a). Finally, surfactants were added in the etchant solution to avoid the gas bubble accumulation and the micropillars formation upon etching. Results showed dramatically reduced photoelectrochemical currents (FIG. 20), suggesting that gas bubble-templating (FIG. 2c) may be critical in forming the light-trapping structures.

### Electrochemical and Photoelectrochemical Characterization

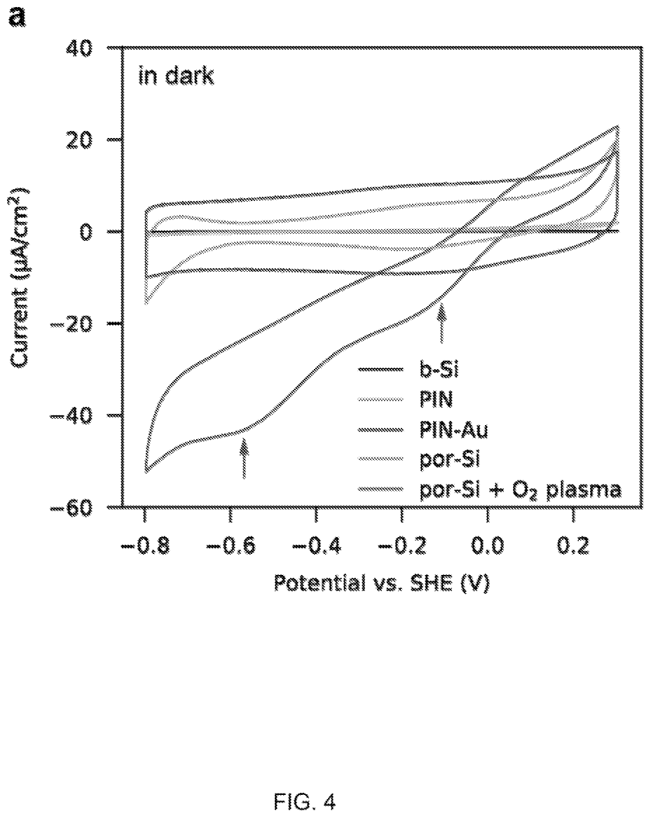

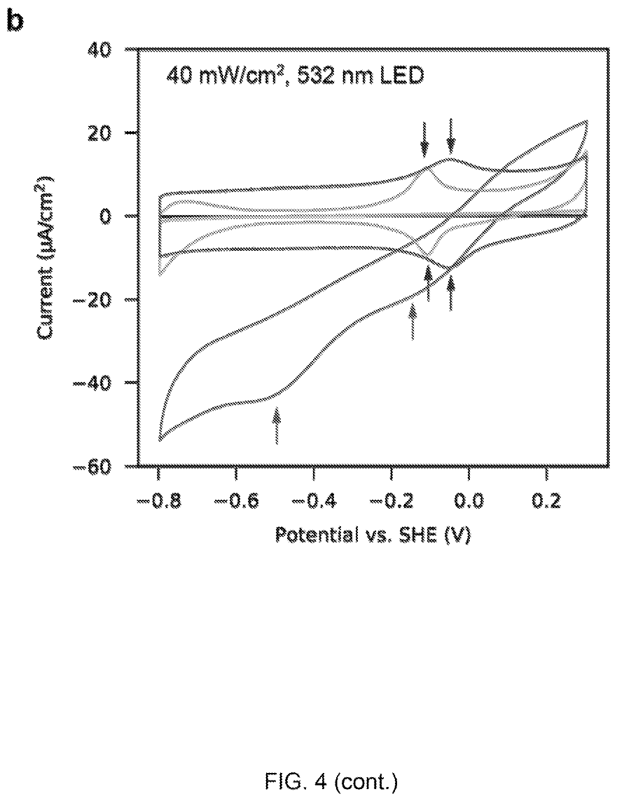

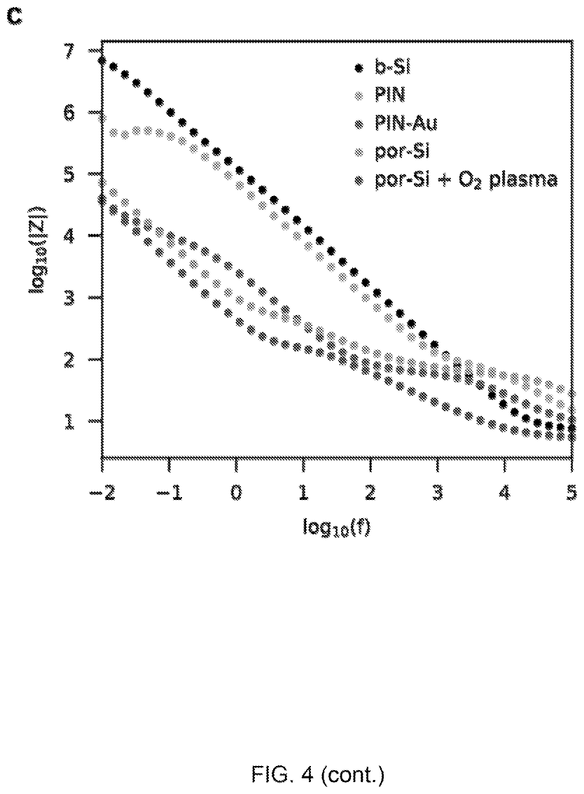

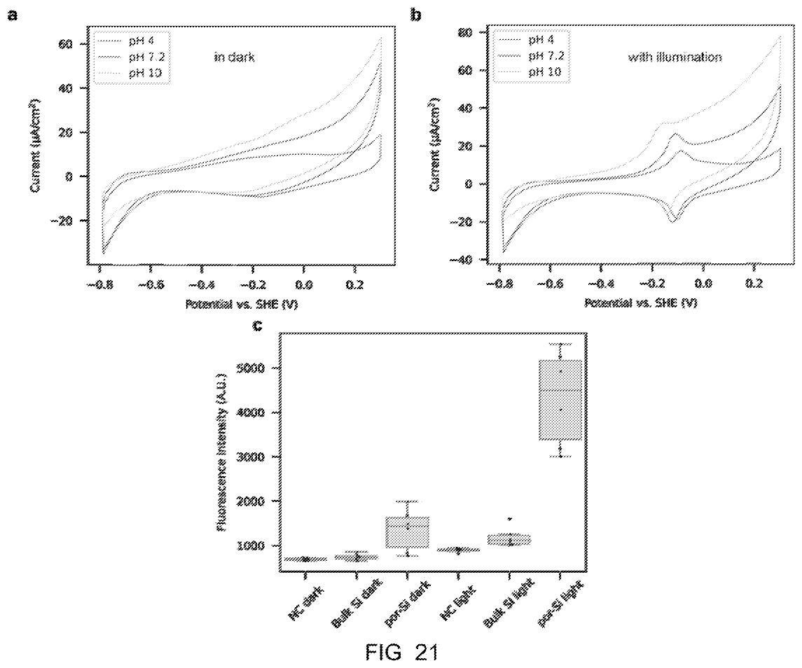

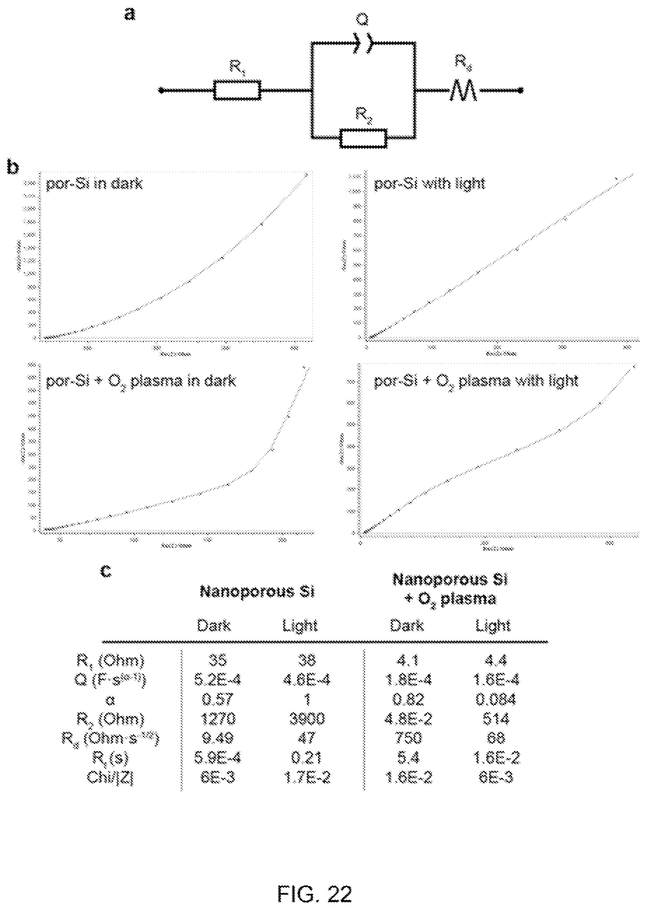

To understand the effects of stain etching and oxygen plasma treatment on the electrochemistry of the silicon, standard electrochemical tests were performed using wired electrodes. FIG. 4a shows cyclic voltammetry (CV) curves of silicon electrodes with different surfaces (such as bulk silicon, stain etched p-type silicon, and p-i-n silicon with or without gold decoration). Stain etching improved the capacitance of the electrode interface over the flat wafer, and oxygen plasma treatment resulted in further enhancement. Additionally, when performing scans with 532 nm light irradiation (FIG. 4b), a pair of reversible-redox peaks were observed, which may be attributed to oxygen reduction reaction on the porous surface³⁷. This photoelectrochemical process is suggested by hydrogen peroxide production as detected through the dye degradation assay (FIG. 21). This electrochemical behavior is functionally different from the previously reported gold-decorated p-i-n junction⁵, which showed strong, but irreversible redox peaks (marked on CV curve in FIG. 4a-b). Furthermore, electrochemical impedance spectroscopy showed that the combination of stain etching and oxygen plasma reduced the impedance of the electrode over the entire range of frequencies (FIG. 4c). Impedance reduction may be attributed to the increase in surface area available for current injection and increased hydrophilicity of the surface, which reduces the barrier for ion transfer to and from the solution. Electrical impedance spectra can be described using Randles equivalent circuit with restricted diffusion element to account for ion transfer through the nanoporous material. (FIG. 22).

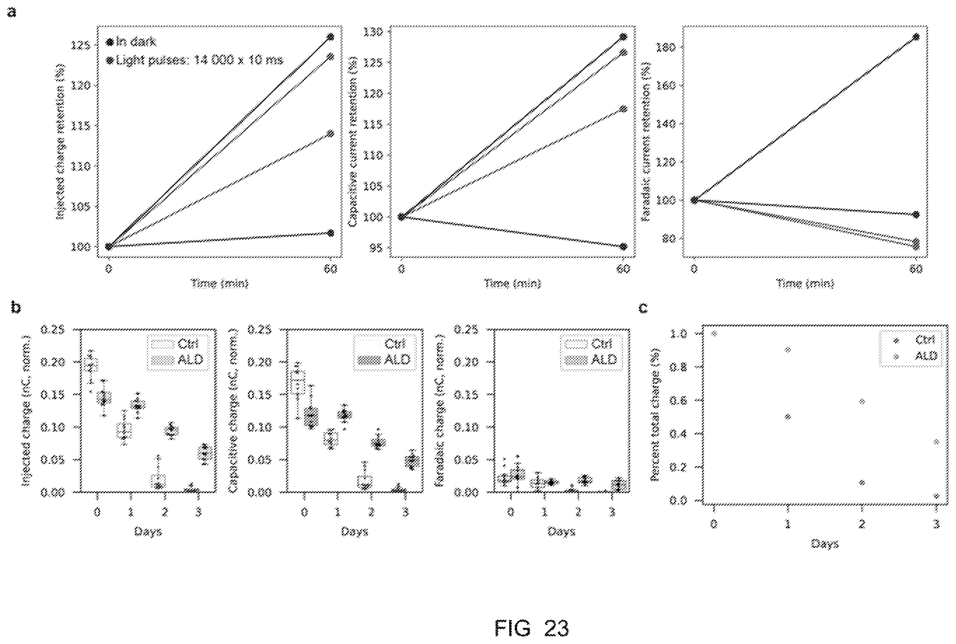

The stability of the silicon heterojunction in phosphate buffered saline (PBS) was studied under illumination. No degradation was found in photocurrent between samples that underwent pulsed illumination of 14,000 pulses and samples stored in the dark for the same time period (FIG. 23). This observation is in sharp contrast to the previously reported gold-decorated p-i-n junction⁵, where a degradation of 30% in generated photocurrents was observed after only 1,000 pulses. The improved device performance supports the CV findings that the photoelectrochemical processes occurring on the nanoporous silicon surface may be more reversible, as opposed to the irreversible redox reaction on the gold-decorated p-i-n junction. Although the nanoporous layer is prone to dissolution in PBS when stored for more than 48 hr (FIG. 23), chemical surface modification or deposition of passivation layers can further improve its stability, similar to stabilisation of photovoltaic cells³⁸. It was found that atomic layer deposition can improve the material stability and increase the lifespan of the photoelectrochemical effects (FIG. 23).

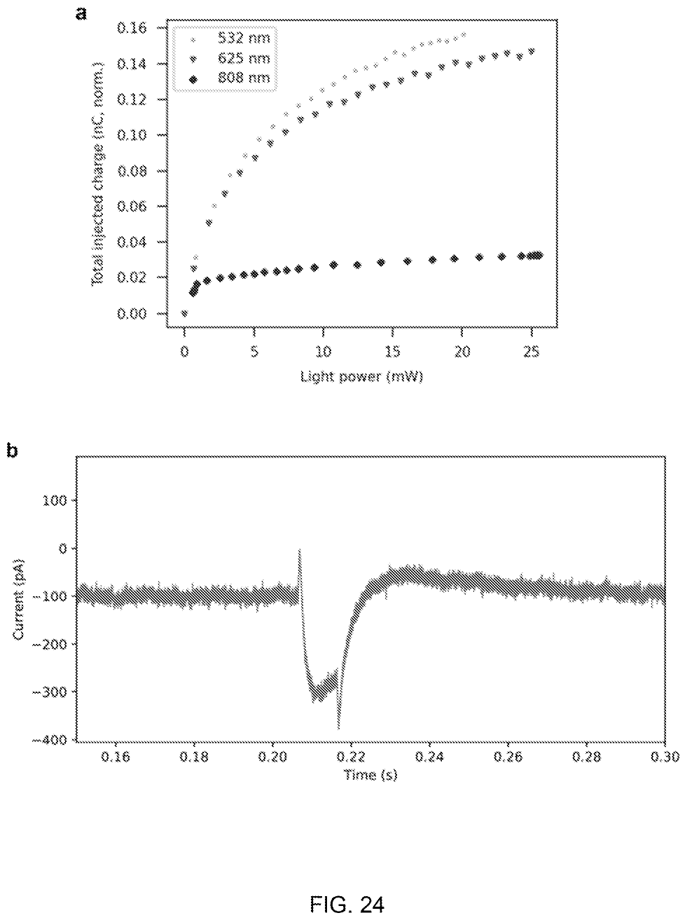

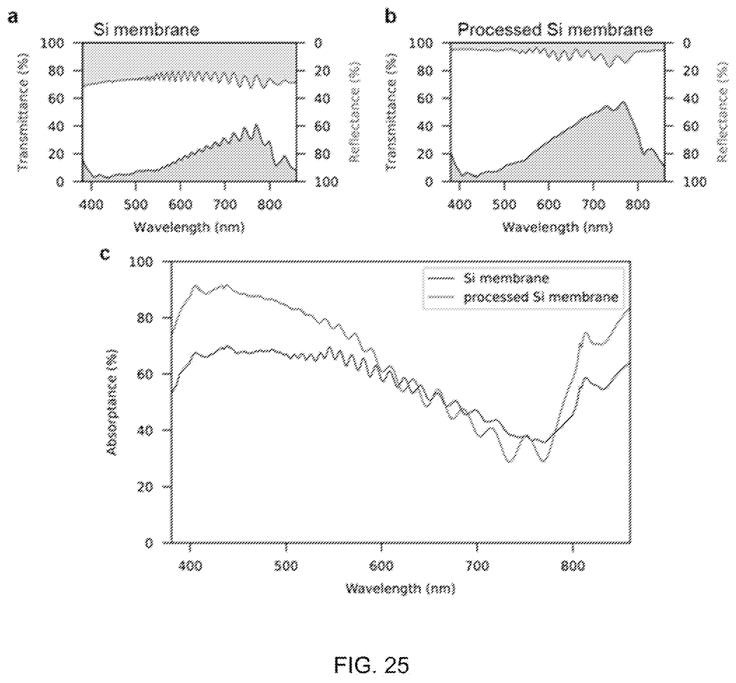

The dependence of the photoelectrochemical currents on the power and wavelength of the illumination light was investigated. While the materials generated large photoelectrochemical currents under visible and NIR light (FIG. 24), ultraviolet (UV) light illumination did not elicit a photoresponse. The absence of photoresponse is attributed to the increased scattering and absorption of UV wavelengths in the nanoporous layer, such that the UV photons cannot reach the buried heterojunction (˜1 μm below the surface) and generate photocarriers. This observation highlights the dominant role of the nanoporous/non-porous heterojunction (that UV photons cannot reach) over that of the nanoporous/saline junction (that UV photons can reach) in producing the observed photoelectrochemical effect. Moreover, although NIR light, which is more attractive for clinical biomodulation applications, was found less effective, it is important to note that the entire stain etching protocol was optimized for 530 nm green light illumination. Thus, it is reasonable to expect that protocol optimized for 808 nm light will yield a better photoresponse for this wavelength. To connect the optical properties of the material with photoelectrochemical performance, microspectrometry of the silicon membranes was performed before and after processing (FIG. 25). It was found that stain etching and oxygen plasma treatment increased transmittance of the silicon membrane, due to removal of part of the material, but reduced reflectance of the membrane. Overall light absorption of the membrane was only slightly increased after complete processing. Given that the recorded photocurrent level is improved by orders-of-magnitude upon stain etching, it is believed that the charge separating diode-like heterojunction (FIG. 1a), as opposed to the nanoporous antireflective layer, is responsible for the enhanced photoelectrochemical properties.

### Ex Vivo Cardiac Modulation

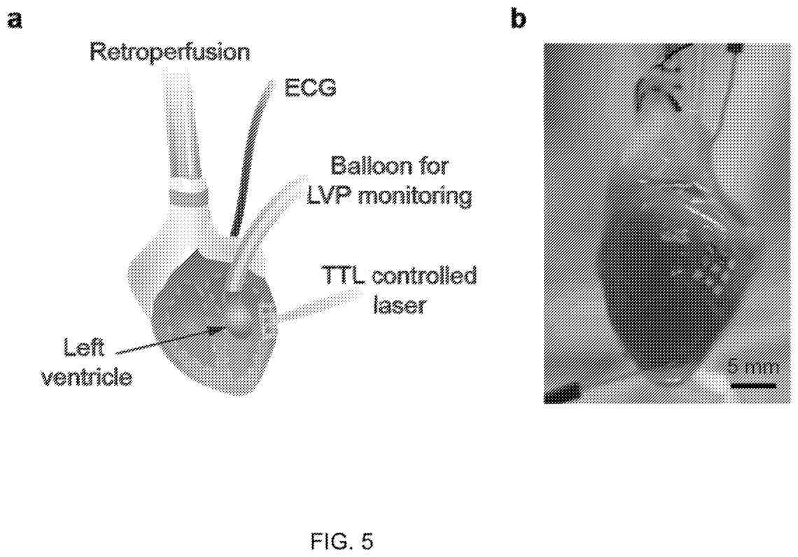

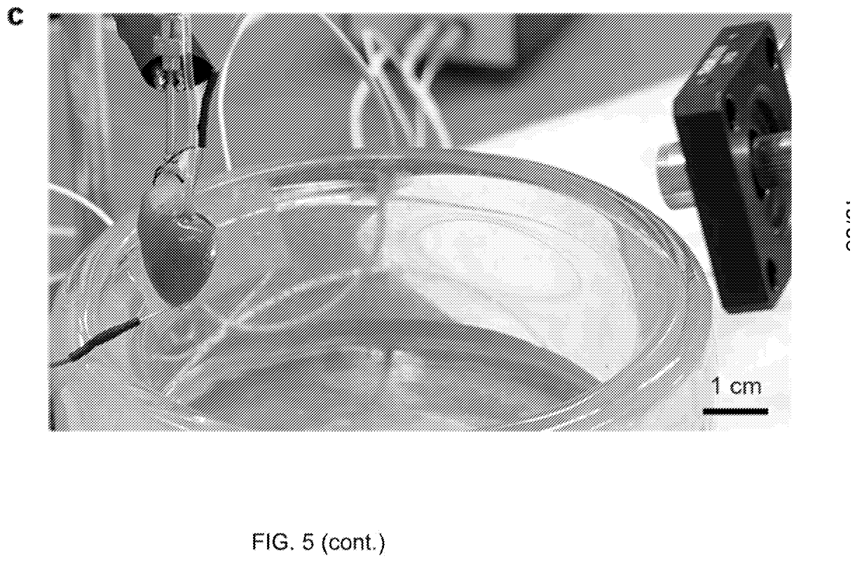

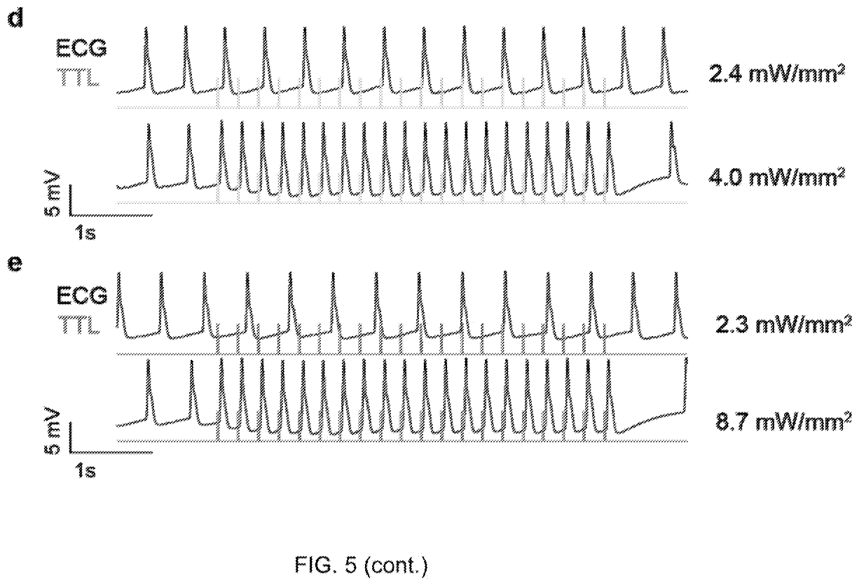

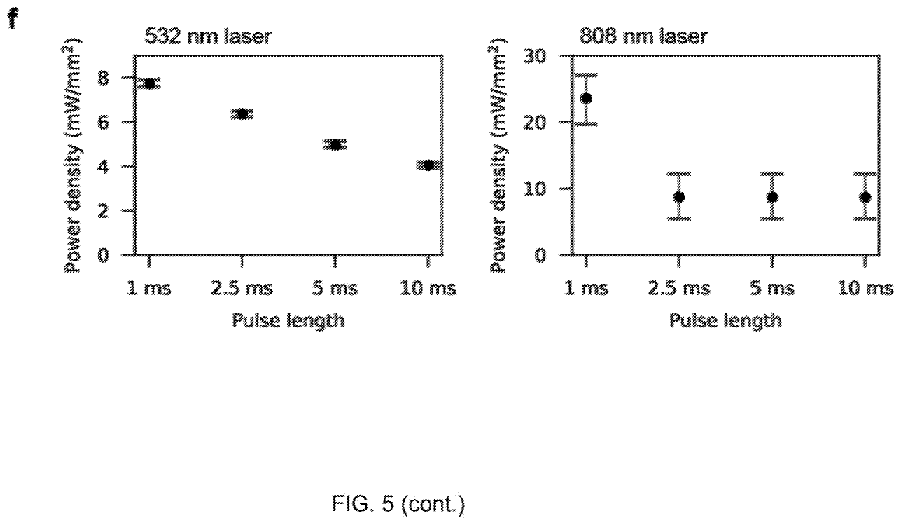

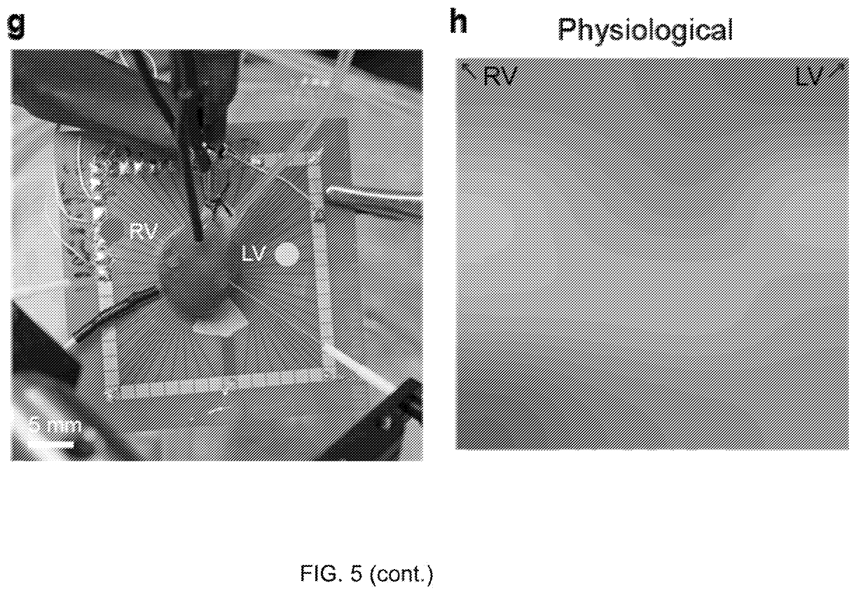

To demonstrate the utility of the free-standing device described herein in converting low optical power into bioelectrical stimulation, the device was interfaced with isolated cardiac tissue in a Langendorff apparatus (FIG. 5a). FIG. 5b shows an image of the device conforming to the soft curvilinear cardiac surface and interfacing with the left ventricle (LV).

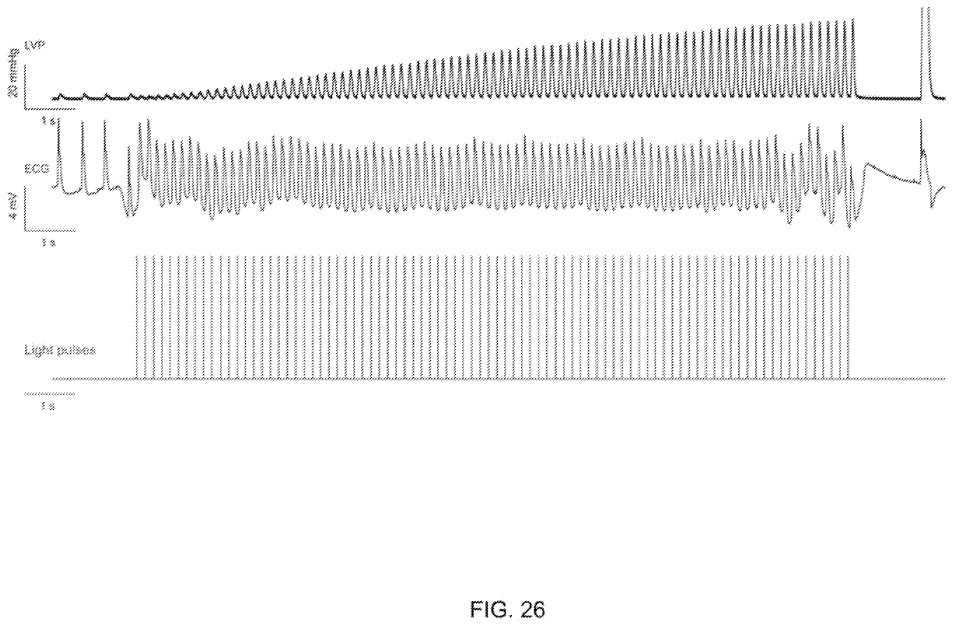

Capillary forces between the flexible membrane and the heart were enough to hold the device in place without any adhesives or physical modifications, despite the wet surface and buffer perfusion. An electrocardiogram (ECG) recording of the electrical activity of the heart showed a slow atrioventricular node rhythm (due to removal of the atriums). Upon application of light pulses (FIG. 5c), the heart immediately synchronized to the frequency of the light pulses (FIG. 5d, FIG. 26) due to effective depolarization of myocardium through injected photoelectrochemical currents. To determine the optical density required for overdrive pacing, a heart was stimulated with 532 nm and 808 nm lasers, as shown in FIGS. 5d and 5e, respectively. It was found that 532 nm light had a lower stimulation threshold than 808 nm light (FIG. 5f). An improved stimulation efficiency over previously reported nanowire-based materials was achieved³⁹; as little as 4 mW/mm² of light (532 nm) was sufficient to stimulate the heart compared to the previously used power of 3.5 kW/mm^{2 39}. The radiant exposure (532 nm) is now on the same order of magnitude as that required for heart stimulation using optogenetics (0.3-2 mJ/cm²)⁴⁰. A notable quality of the device disclosed herein is the ability to utilize NIR wavelengths (808 nm) for optical biomodulation which can penetrate into deep tissues⁴¹. This is another meaningful advantage over optogenetics, in which opsins have a distinct light absorption band limited to visible light with low tissue penetration capabilities. Moreover, the radiant exposure necessary to stimulate the heart with NIR light (˜2 mJ/cm²) was also significantly lower than that required for the unaided IR stimulation of embryonic hearts (800 mJ/cm²)¹⁵.

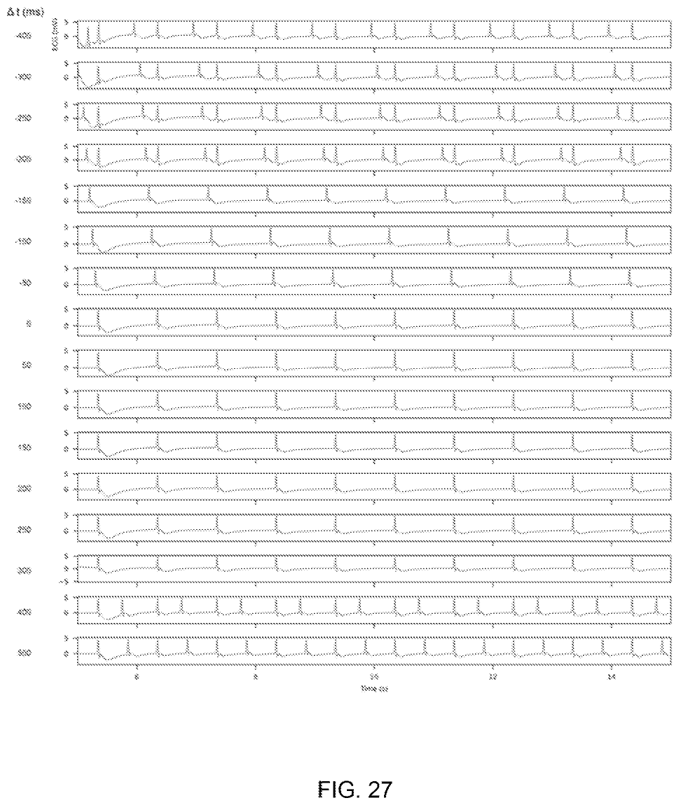

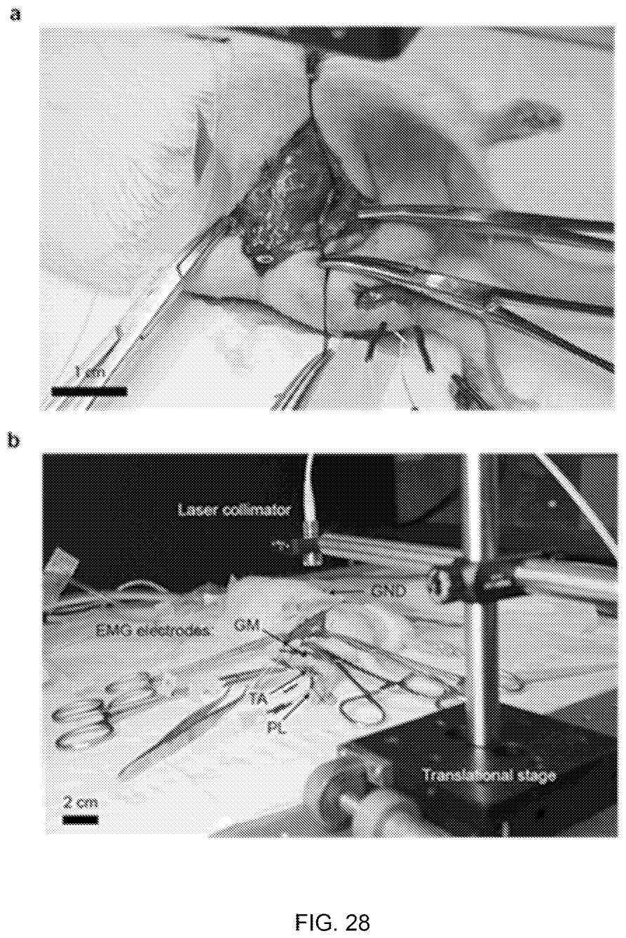

As the device described herein is completely free-standing, it can be positioned at any location on the heart, free of lead-associated limitations. This suggests that the device can be used for simultaneous stimulation at multiple sites for cardiac resynchronisation therapy, similar to previously reported work¹⁹, but without the need for genetic modifications. To demonstrate the applicability of dual chamber pacing, experiments were performed using two devices, one placed on the LV wall and the other on the right ventricular (RV) wall (FIG. 5g). Additionally, a microelectrode array was used to map the field potential propagation throughout the myocardium, and observed different propagation waves under varying stimulation conditions (FIG. 5h). Two separate contractions were observed only when sufficient time delay between the two pulses was used (FIG. 27), which also depends on which pulse is leading in the stimulation. It is believed that this preliminary result shows potential for application of the device in heart stimulations from multiple positions, which increases its versatility and therapeutic potential.

### In Vivo Sciatic Nerve Modulation

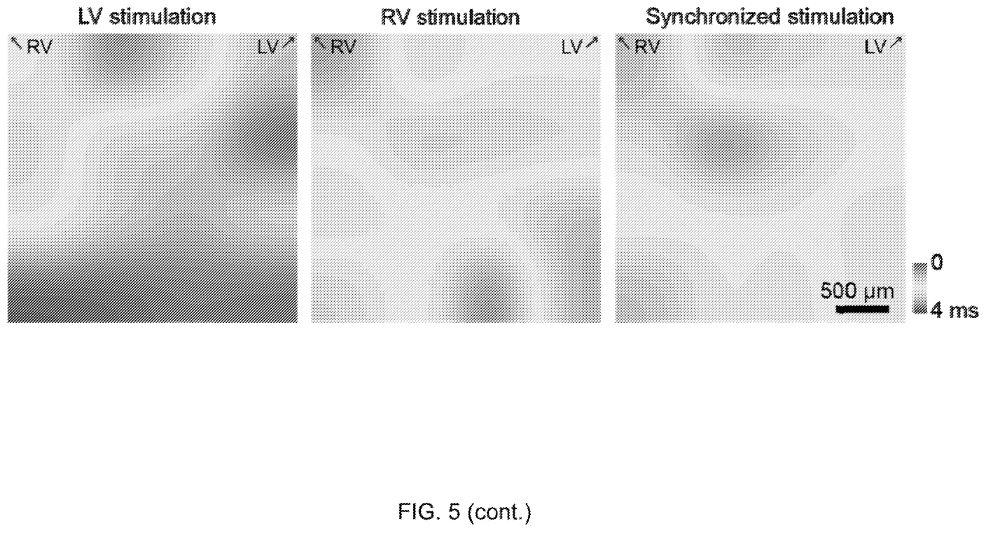

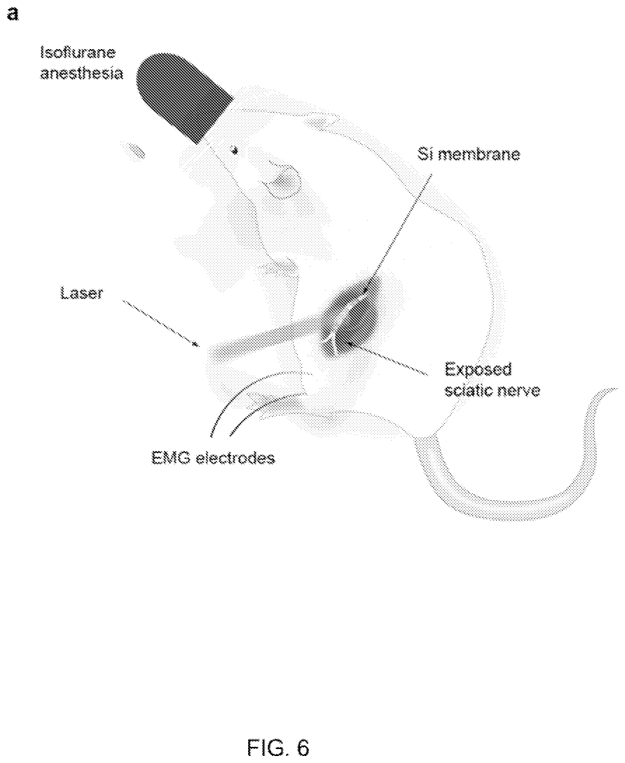

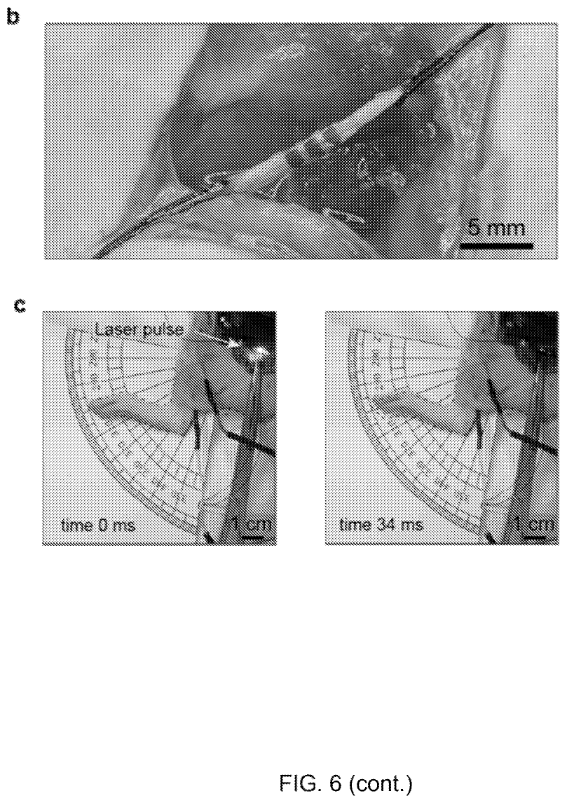

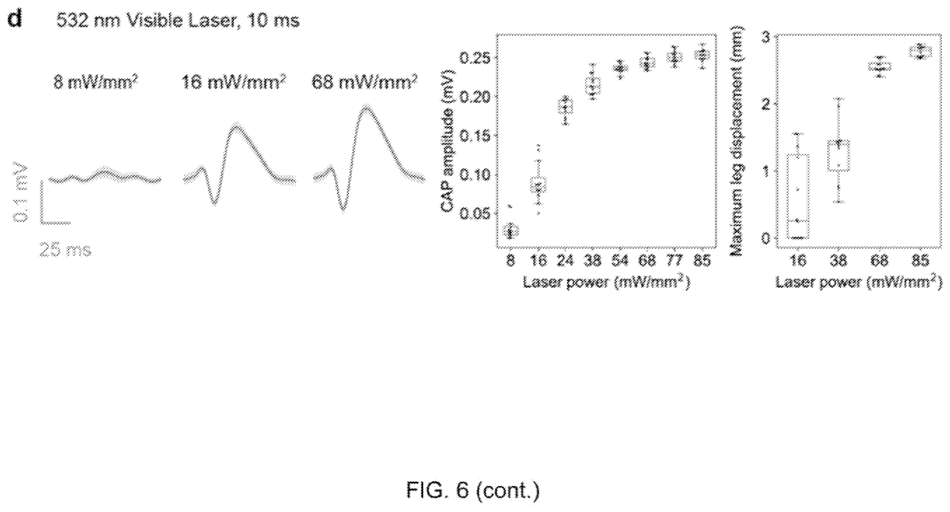

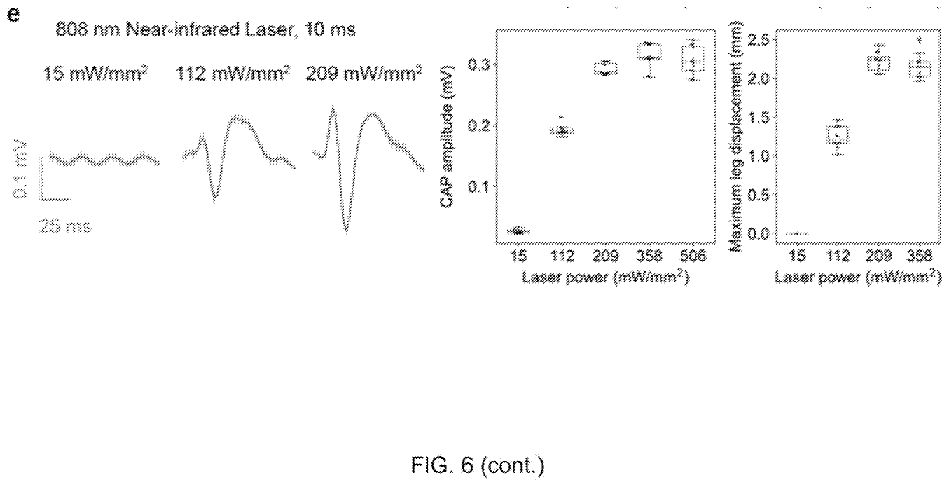

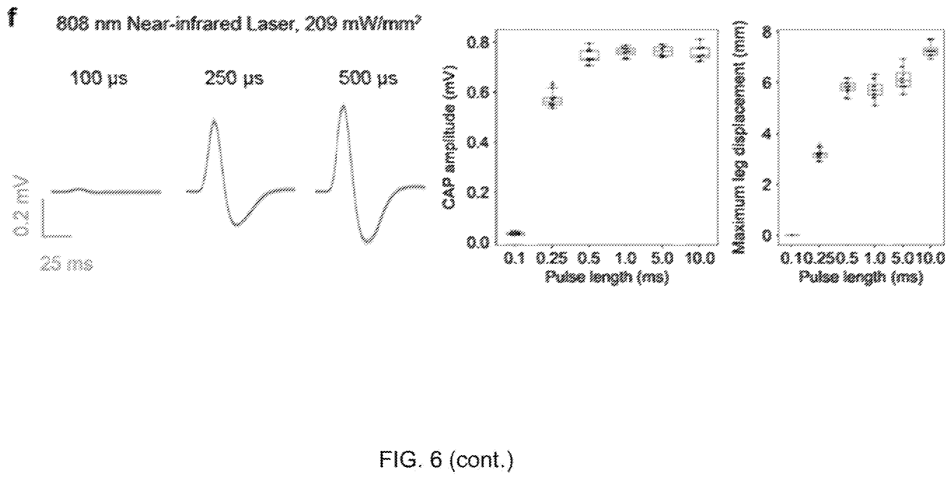

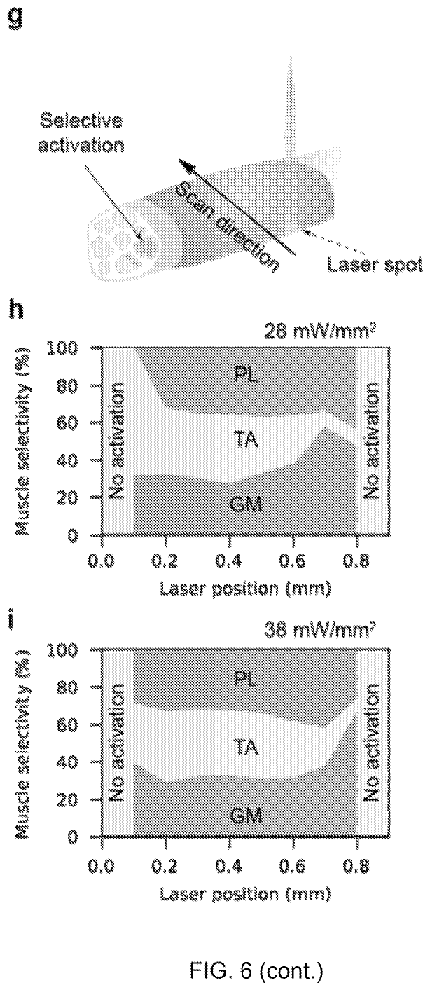

Finally, the utility of the device in neuroregenerative applications was demonstrated and studied acute in vivo sciatic nerve biomodulation in rats (FIG. 6a). FIG. 6b shows that the flexible device can wrap around the exposed sciatic nerve without any apparent breakage and can adhere to it without need for glue or suture. Illuminating the device with pulsed light resulted in an action potential (AP) that propagated through the nerve and moved the associated lower limb (FIG. 6c). In contrast to heart stimulation, where binary, sub- or supra-threshold pacing effects were observed, the sciatic nerve showed an intensity-dependent effect. Stimulation of the nerve was investigated using 532 and 808 nm lasers and measured compound action potential (CAP) amplitude and maximal leg displacement. FIG. 6d-e shows a correlation between the optical power densities and the pulse duration to the CAP amplitude and leg displacement, respectively.

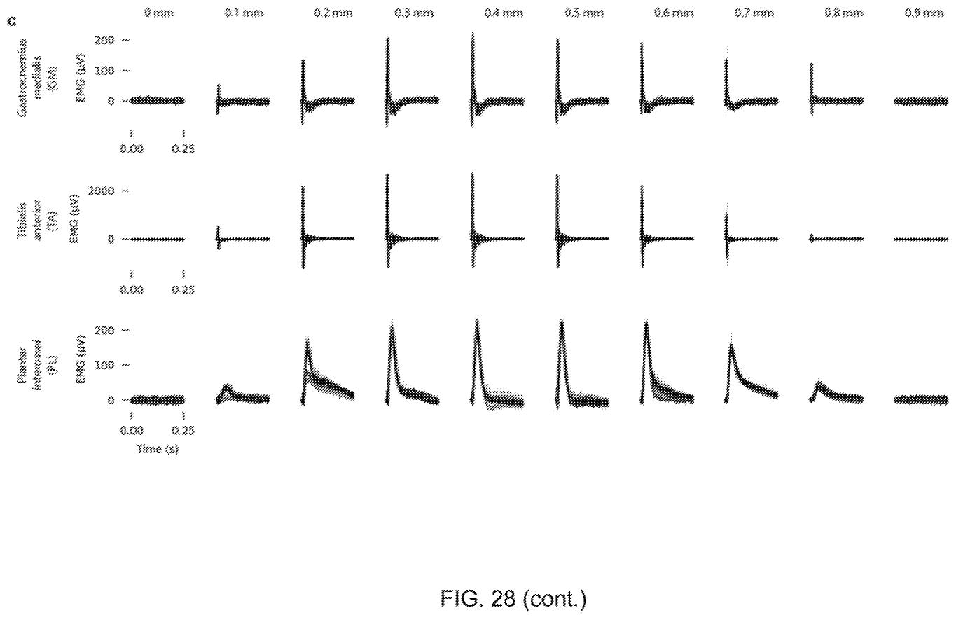

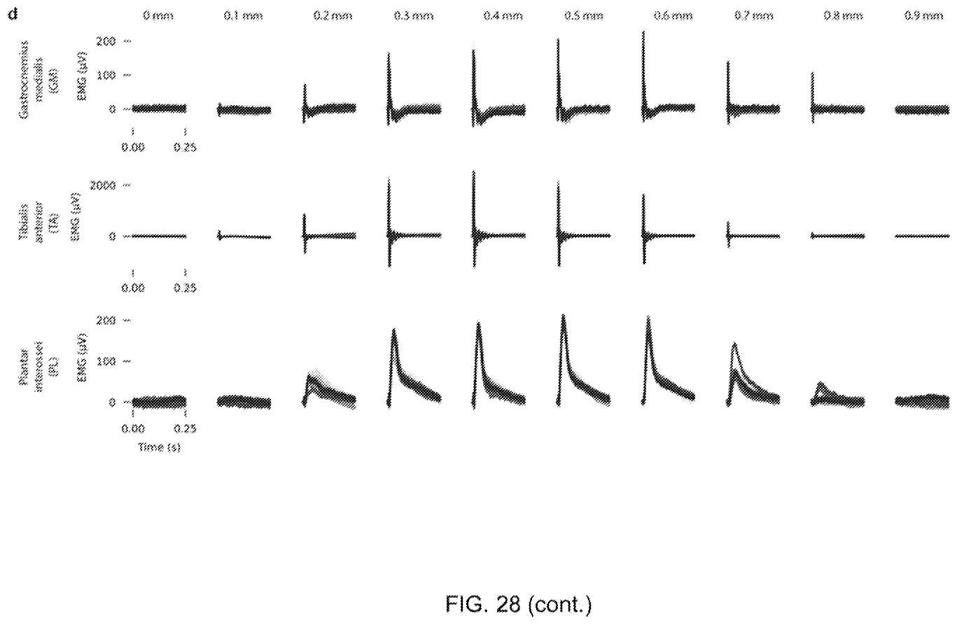

In this context, it is important to realize that the sciatic nerve is a bundle of nerves⁴²; consequently, the different nerves within the sciatic bundle may have different stimulation thresholds and/or different distances from the silicon membrane. Thus, the intensity dependence of the optical stimulation may be attributed to the ability of higher electrical intensity to stimulate nerves with higher thresholds and penetrate deeper into the bundle. Multichannel electromyography (EMG) recording was used from gastrocnemius medialis, tibialis anterior, and plantar interossei muscles to study the activation selectivity (FIG. 6g, FIG. 28). By adjusting power and position of the laser beam (FIG. 6h-i), different muscles were able to be activated to a varying extent. In clinical application, controlling the intensity and selectivity allows tuning the stimulation to yield a therapeutic output while minimizing pain and discomfort. Additionally, the selectivity reduces inverse recruitment of muscle groups, which delays the onset of muscle fatigue⁴³. While silicon membranes are not as selective as multipolar cuff and intrafascicular implants^{44,45}, the improvement in membrane pattern design and application of beams with smaller diameters may further increase selectivity. Finally, the limb was successfully stimulated with a pulse as short as 500 s for NIR laser which corresponds to a radiant exposure of 10 mJ/cm², an exposure that is energetically efficient and safe for most tissues.

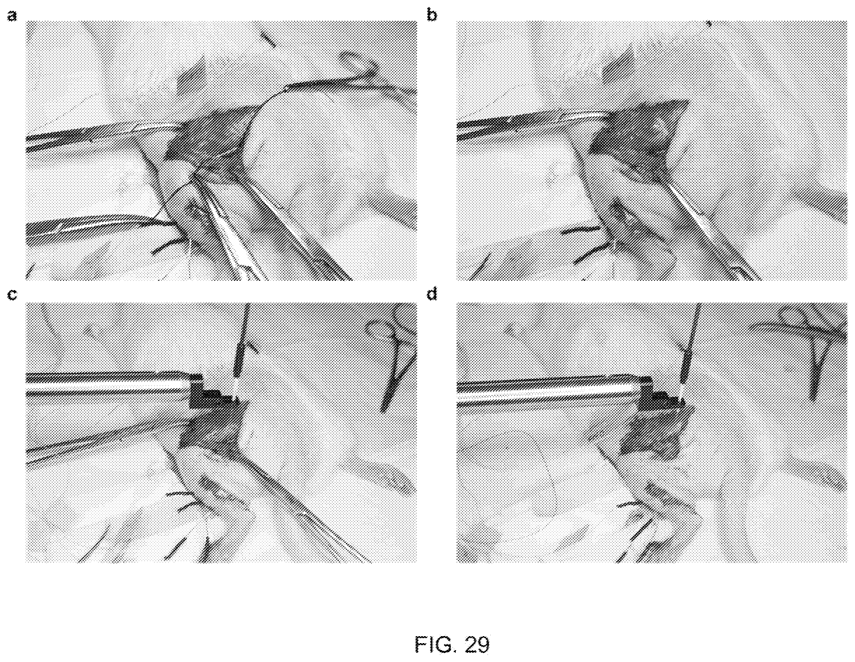

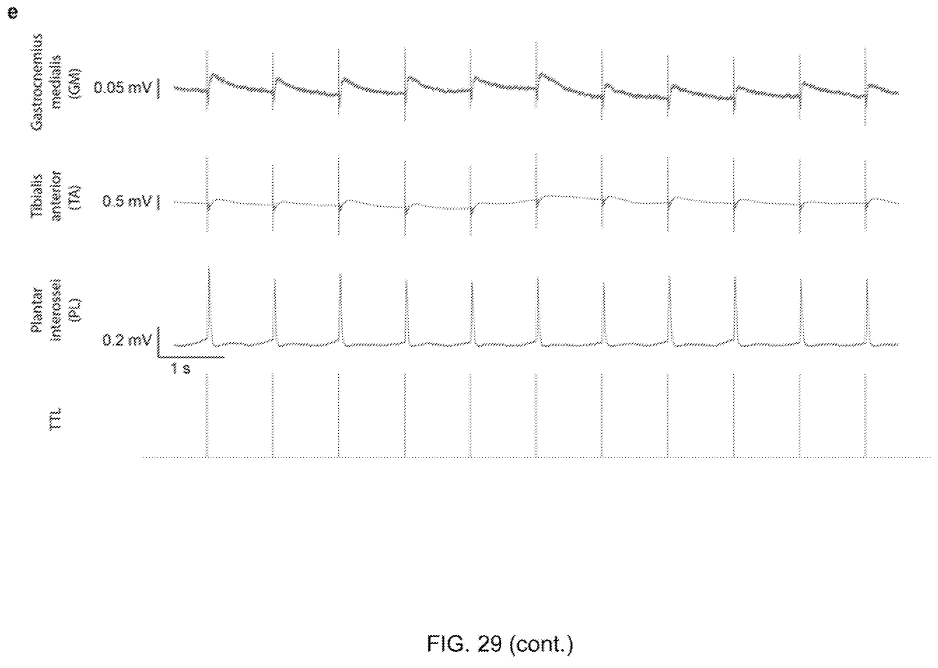

While the experiments described herein operate on the exposed nerve, the method is easily translatable to standard post-operative stimulation methods that employ implanted optical fiber cannulas (FIG. 29). Although in this proof of concept a glass fiber cannula was used, soft and flexible hydrogel-based optical fibers⁴⁶ can be used to deliver light to the interfacing nerve in vivo. Additionally, deep red- and NIR-induced photocurrent generation may enable fully remote transdermal stimulation using an NIR transmission window⁴¹. Moreover, the thin PDMS used here as a support layer for the silicon membrane can be avoided or replaced with hydrogel-based conduits to improve adhesion and retention of the electrode after wound closure.

### CONCLUSIONS

The approach described in this study combines fabrication simplicity with high functional efficiency. Thus, it is believed that the porosity-based heterojunction is a promising candidate for future translational research on bioelectronic therapies. The disclosed methods enables fabrication without sophisticated instrumentation allowing researchers to develop optoelectronic devices for biomedical applications cost-efficiently. Moreover, the use of pristine p-type silicon, which is broadly regarded as biocompatible, for the main building block of the optoelectronic stimulation device is an advantage from a regulatory perspective as an application of multi-material composites introduces additional safety concerns due to cross-talk between components. Although the instability of the material may pose some limitations, preliminary surface modification experiments showed promising results that suggest that the stabilization of the materials can be easily tuned to meet the desired therapeutic time scale.

### Methods

### Stain Etching of Wafers and Membranes

Silicon wafers (P<100>0.001-0.005 ohm·cm, N<100>0.001-0.005 ohm·cm, I<100>>10,000 ohm·cm) were obtained from Nova Electronic Materials (Flower Mound, TX, USA). The wafers were diced using the Disco DAD3240 Dicing Saw. 5 mm×5 mm squared pieces were used in photocurrent evaluation experiments and 10 mm×10 mm squared pieces were used for fabrication of electrodes used in photoelectrochemical measurements and fluorescence measurements. PTFE tweezers were used for all sample manipulation, from initial to final washing steps. Directly before etching, wafers were washed in an ultrasonic bath for 5 min in acetone (Histological grade, A16P-4, Fisher) followed by 5 min in isopropanol (Certified ACS Plus, A416P-4, Fisher), and then stored in deionized water (DI water, 18.2 MOhm, Micropure UV/UF, ThermoFisher). All stain etching experiments were performed in concentrated hydrofluoric acid (HF, TraceMetal grade, A513-500, Fisher). Concentrated nitric acid (HNO₃, TraceMetal grade, A509-P500, Fisher), iron(Ill) chloride hexahydrate (FeCl₃, ACS reagent, 97%, 236489-500G, Sigma Aldrich), and vanadium(V) oxide (V₂O₅, 99.6+%, 206422500, Acros Organics) were used as porous silicon-forming oxidizers. Sodium dodecyl sulfate (SDS, BioReagent, ≥98.5% (GC), L3771-25G, Sigma Aldrich), Pluronic® P123 (435465-250ML, Sigma Aldrich) and Tween® 20 (P2887-100ML, Sigma Aldrich) were used as surfactant additives. Solutions and chemicals were used as received without additional processing and all experiments were performed at room temperature (24-26° C.). Directly before etching, HNO₃ was mixed with HF in specified v/v ratios, or FeCl3, V2O5 and surfactants were dissolved at specified concentrations and solution was deoxygenated for 30 min by bubbling with argon. Each etching series (a set of samples using the same etchant composition and time) used approximately 10 mL of freshly prepared etchant and was performed in a fresh polypropylene dish. Silicon wafer samples were transferred from water bath to etching solution, moved along the bottom of the dish with the tweezers for the first 10 seconds, and left undisturbed for the specified amount of time.

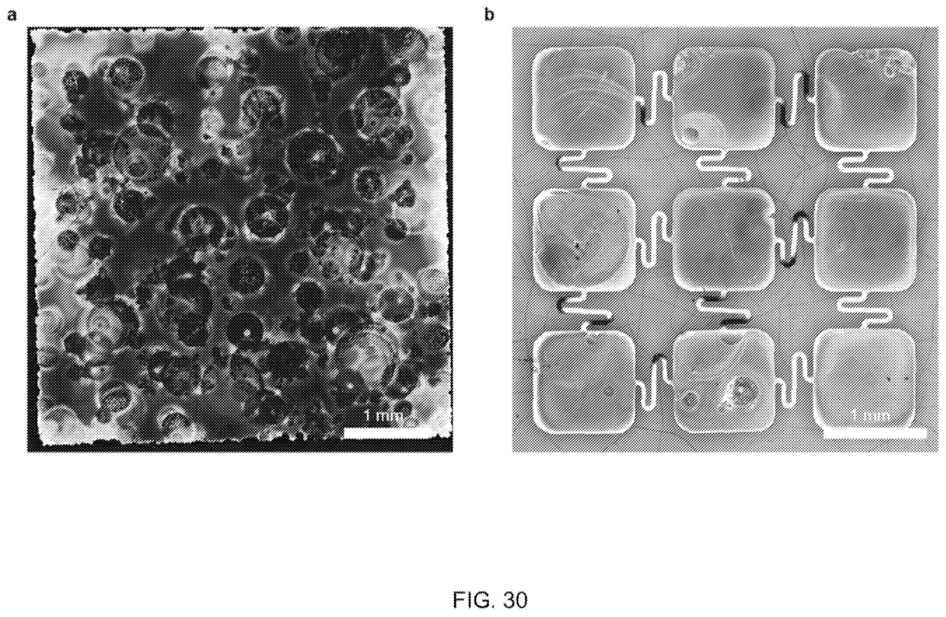

After etching, samples were soaked twice for 1 min in separate DI water baths to remove the HF, once for 1 min in IPA, and dried using compressed nitrogen. 5 mm×5 mm samples were mounted on reclaimed grade silicon wafers using double sided tape to ease further handling, and silicon membranes were transferred and attached to PDMS substrate using fresh wet oxide silicon wafers. PDMS casting solution was prepared by mixing PDMS base (Sylgard™ 184, Dow), PDMS cure (Sylgard™ 184, Dow) and hexane (anhydrous, 95%, 296090-1L, Sigma-Aldrich) in a 10:1:10 (w/w) ratio. The solution was then degassed under vacuum, spin coated at 4,000 rpm onto the PMMA covered microscope slide and cured at 65° C. for 45 min. All samples were stored in a desiccator under vacuum for prolonged periods between experiments (>2 h). FIG. 30 shows optical microscopy photographs demonstrating formation of colorful stains on the surface of the wafers and silicon membranes. Porous bulk wafer appears mostly black under the reflected light and the silicon membrane shows iridescent stains due to thin-film interference. It is important to note that the difference in optical appearance is due to different substrate thicknesses and not different etching conditions.

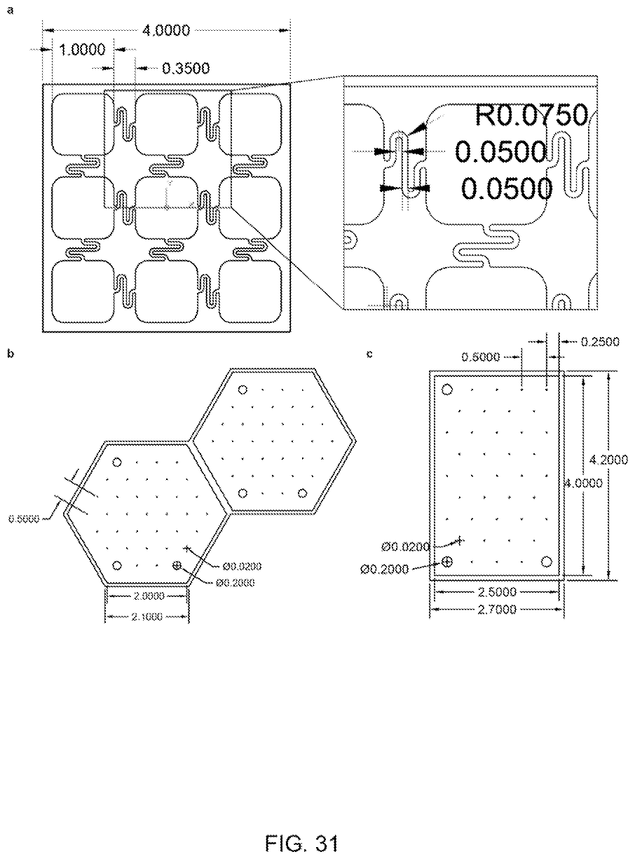

Silicon membrane fabrication. Silicon-on-insulator (SOI) wafers (p++, device 2 μm, 0.001-0.002 ohm·cm, handle 10-100 ohm·cm, BOX 1 μm) were obtained from Ultrasil (Hayward, USA). Photomask was designed using AutoCAD software and is shown in FIG. 31. Fabrication followed standard lithography procedures. A thick positive resist AZ40XT-11D (MicroChemicals) was used as a mask for reactive ion etching. Patterns were exposed using the Heidelberg MLA150 direct writer and uncovered SOI was removed using SF6/CHF3 (20 sccm/50 sccm) RIE (ICP 600 W, RF 60 W) in a Plasma-Therm ICP Fluoride etcher with an etching rate of about 600 nm/min. Photoresist was stripped using AZ NMP (MicroChemicals) and wafers were washed in an ultrasonic bath for 5 min in acetone followed by 5 min IPA and dried using compressed nitrogen. Samples were submerged in concentrated HF (analytical grade, 48 to 51% in water, 223335000, Acros Organics) for 4-8 h to remove buried oxide layers. The membranes, together with the handle wafer, were subsequently soaked twice for 1 min in separate DI water baths to wash off the HF. The wafers were soaked in IPA bath for 1 min and moved to an acetone bath to ease interactions between membranes and handle wafer. Finally, the free-floating membranes were transferred to a fresh IPA bath using separate wet oxide silicon wafers and stored at 4° C. before further processing.

Oxygen plasma processing. Stain etched silicon samples were treated with 800 W oxygen plasma at 50 sccm without RF bias for 10 min in a Plasma-Therm ICP Fluoride etcher. Free samples of silicon wafers were directly attached to the carrier wafers using vacuum mounting oil, and mounted samples and silicon membranes were attached with their respective carrier substrates.

P-type/intrinsic/N-type (PIN) diode junction synthesis. PIN junctions were synthesized through chemical vapor deposition (CVD) growth of intrinsic and n-type layers on p-type silicon wafers as previously described⁴⁰. Gold-decoration was performed for 1 min at room temperature in a solution of 1 mL 10 mM chloroauric acid (16583, Electron Microscopy Sciences), 9 mL DIW, and 0.2 mL concentrated HF (Acros Organics).

### Hydrogen Peroxide Production Assays Under Ambient Conditions

Sample pieces (5 mm×5 mm) were placed in plastic cuvettes with a frontside facing up. The aqueous solution was freshly prepared right before the experiment and contained horseradish peroxidase (HRP; 4250 units/L, Sigma) and (p-hydroxyphenyl) acetic acid (p-HPA, 1×10³ M, Sigma) in Tris buffer (0.25 M). Samples submerged in 0.5 mL of PBS solution was placed in the cuvettes for 20 min. Xe arc lamp at the power intensity of 150 mW/cm² was used for sample irradiation. After the exposure, 0.1 mL of the solution was transferred to the black-walled 96-well plate, 0.1 mL of enzyme solution was added, and was incubated for 10 min. Fluorescence intensity was evaluated using a plate read (Synergy Neo HTS). Fluorescence signals were measured by an excitation wavelength of 320 nm and an emission wavelength of 405 nm.

Photocurrent measurements. Photocurrent measurements generally followed previously described methods³⁵. Patch clamp-setup integrated with an upright microscope (Olympus, BX61WI) with a 20×/0.5 numerical aperture water-immersion objective was used for photocurrent measurements. Light pulses were delivered using episcopic illumination with a dichroic mirror (for 365 nm, 532 nm, and 625 nm LEDs, FF660-Di02-25×36, Semrock) or Thorlabs protected silver mirrors (for 808 nm laser, PF10-03-P01, PFR10-P01). Applied light sources and their validation is described in a separate subsection. Clamp voltage and current were measured using silver chloride electrodes and amplified using an AxoPatch 200B amplifier (Molecular Devices). Voltage-clamp levels and light pulsing were digitally controlled using transistor-transistor logic (TTL) or analog signals delivered from a Digidata 1550 digitizer (Molecular Devices) controlled with Clampex software (Molecular Devices). Glass pipettes were pulled in a P-97 micropipette puller (Sutter Instrument) usually to a resistance of 1-4 MΩ and filled with 1×PBS. In a typical measurement, the material was placed in a petri dish filled with 1×PBS and positioned in the center of the field-of-view. The pipette tip was lowered close to the surface of the material (<10 μm) and the measurement sequence was executed. The measurement sequence spans 400 ms, with a first voltage level of−0.5 mV between 100-300 ms. At 200 ms, a 10 ms light pulse was delivered to elicit a photoresponse from the material. The holding current was adjusted so that the current in the first voltage level was close to 0 pA (usually under 2000 pA). Following the measurements, all samples were soaked in DI water for 1 min, washed with DI water, washed with IPA, dried using compressed nitrogen, and stored under vacuum or immediately used for further experiments.

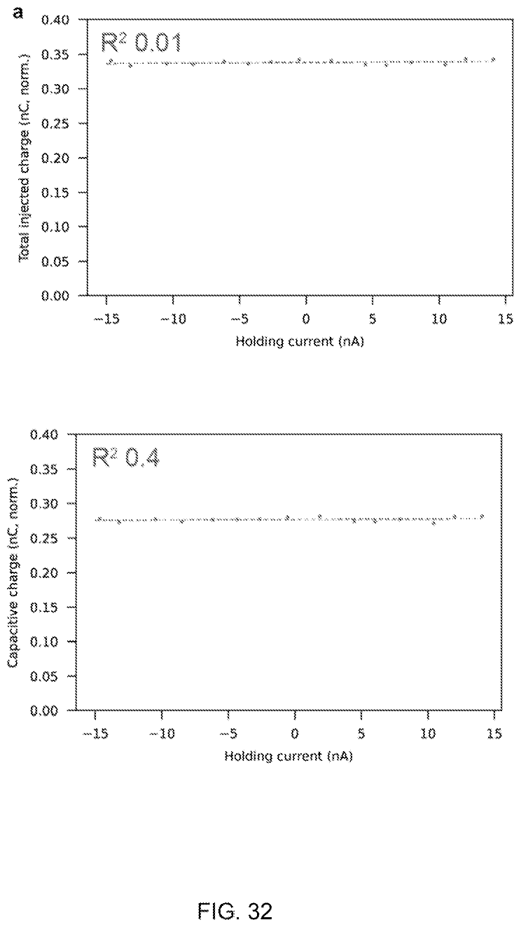

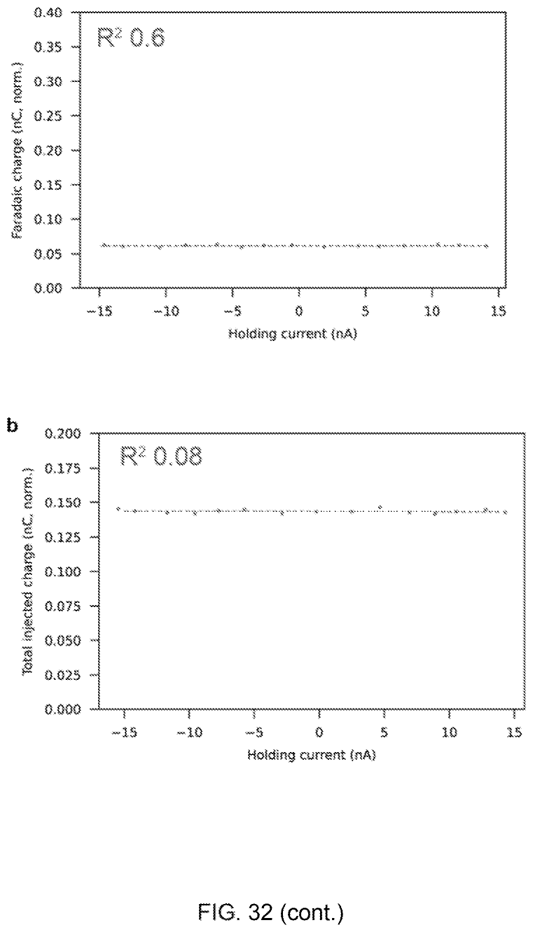

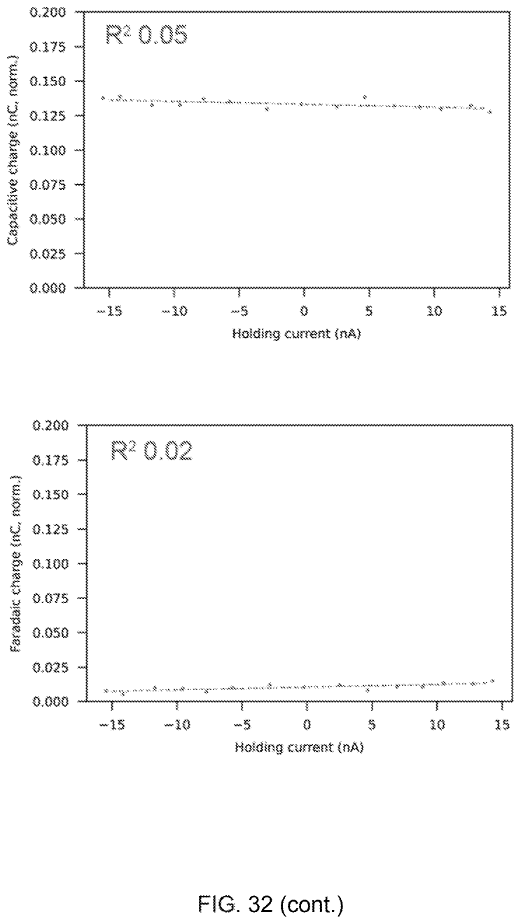

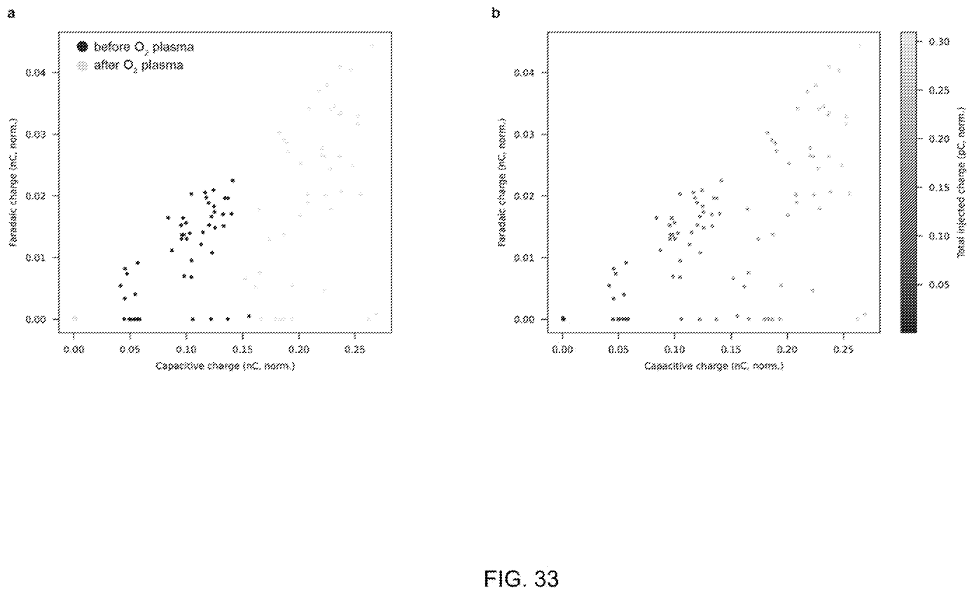

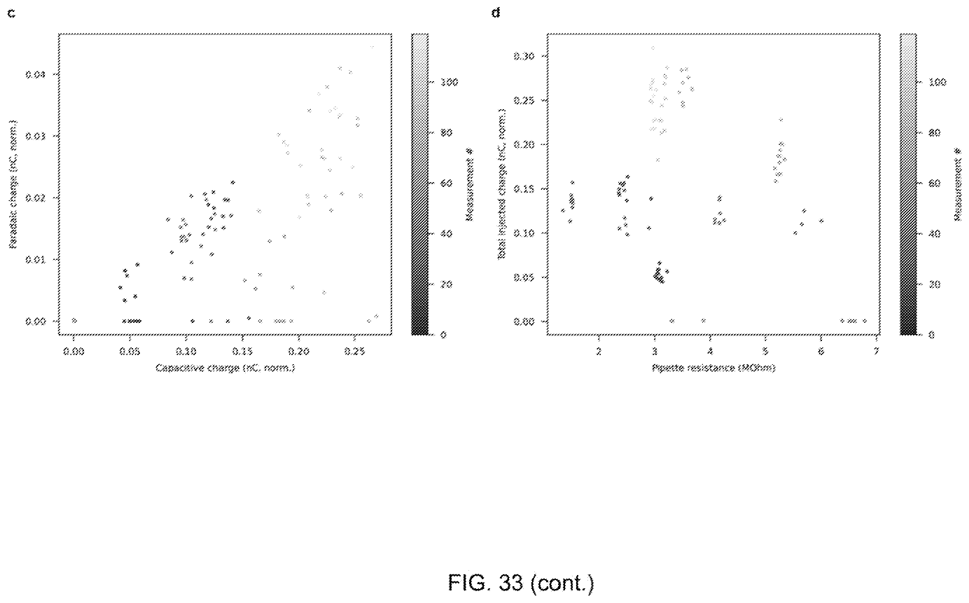

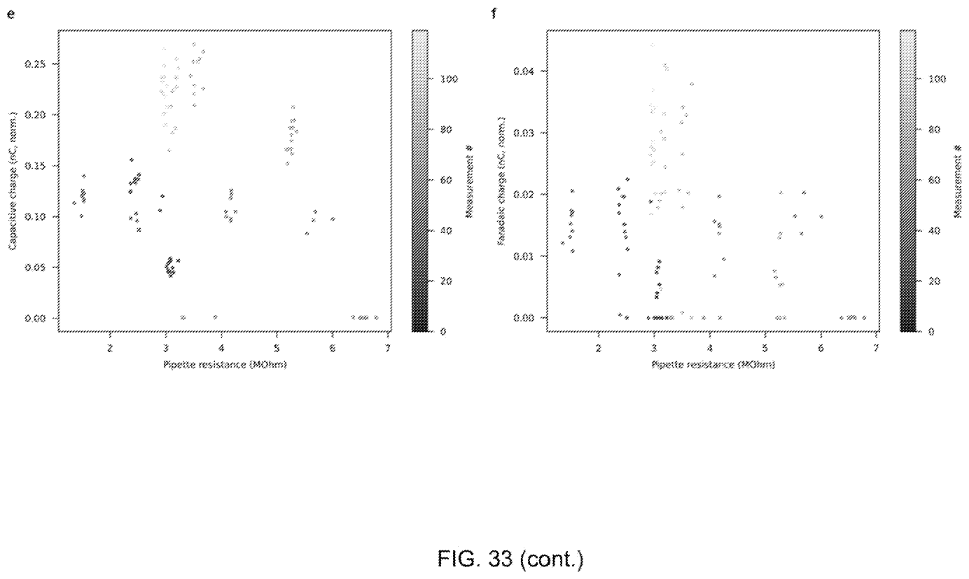

Photocurrent analysis. Photocurrents were analysed with Python script working directly on the raw data files using pyABF library. Total injected charge Qₜₒₜ was calculated through the integration of the area under the current transient during light illumination. Due to high capacitive photocurrents in the material, the faradaic charge was calculated through fitting the last 20% of the current transient to the exponential decay equation: I(t)=A exp^{kt}+I_{f}, where I(t) is current, t is time, I_{f} is the faradaic current, and A and k are constants. Poor quality fits were obtained if more of the transient was used for calculations due to supercapacitive behavior of the material in early charging stages that does not follow exponential trend. Non-linear solver using Trust Region Reflective algorithm implemented in Scipy python package was used for fitting. Faradaic current is assumed to stay constant during the illumination and was used to calculate faradaic charge Q_{f}=I_{f}Δt. Finally, the capacitive charge was calculated by subtracting faradaic charge from total injected charge Q_{c}=Qₜₒₜ−Q_{f}. All recorded values are normalized to measurement performed with 1 MΩ pipettes according to Ohm's law. The detected photocurrents were exported into the delimited text files for further analysis and plotting. Raw data and analysis scripts are available in an online repository. Only measurements that failed due to technical reasons (such as broken pipette, or light pulse not fired) were excluded from analysis. An unsatisfactory photoresponse was not considered a valid reason for a datapoint rejection. Photothermal currents in wafer and membrane samples were not detected (FIG. 32), and therefore they are ignored in the general analysis. Additional validation of the photocurrent analytical methods is described in FIG. 39.

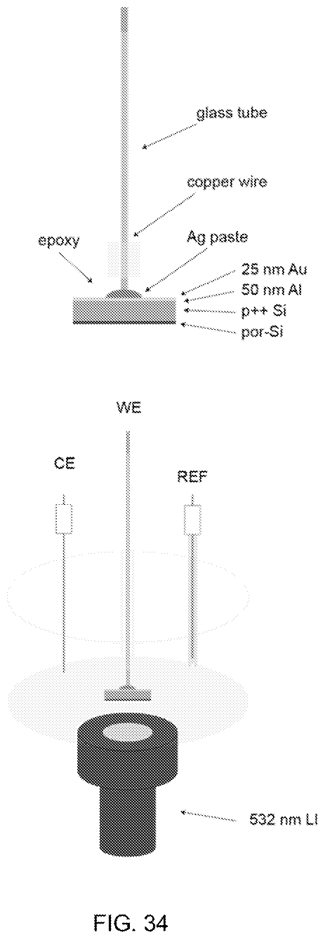

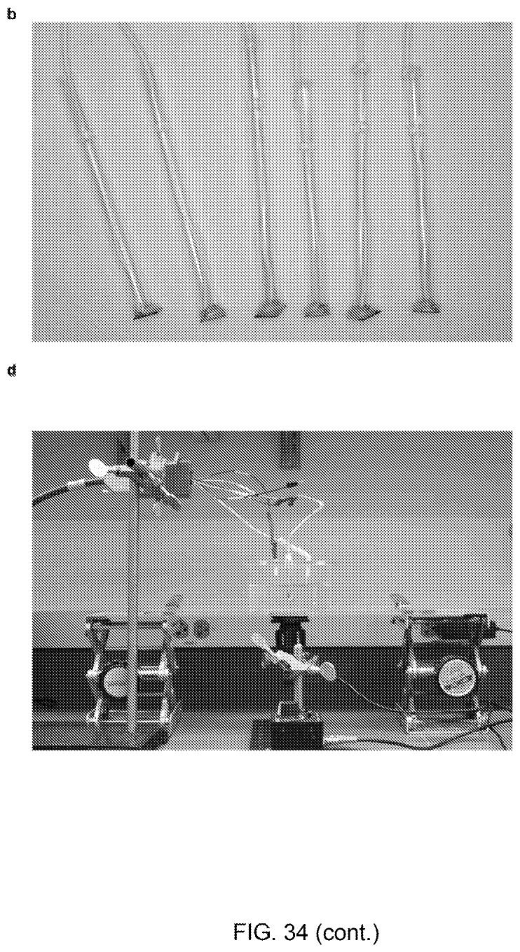

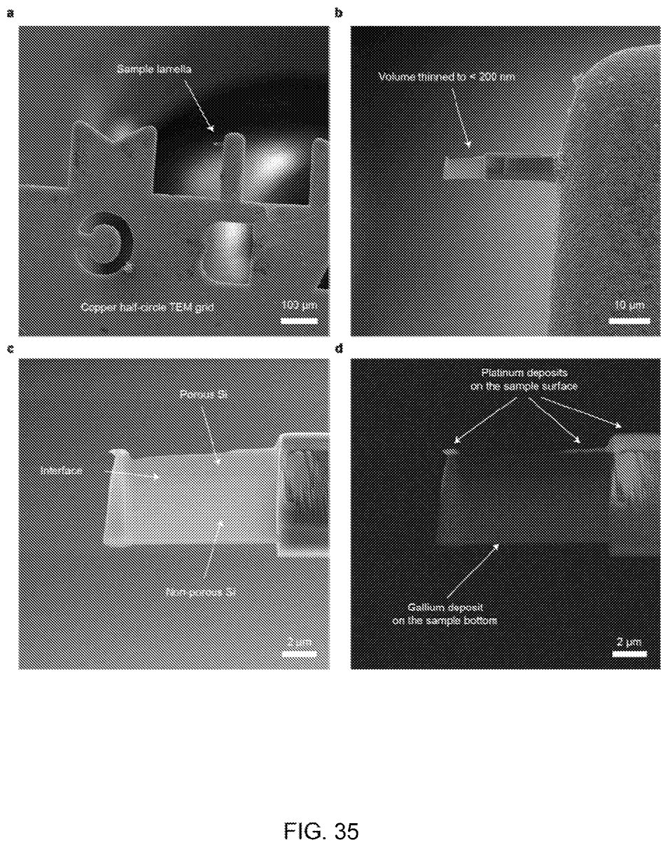

Electrode fabrication. 10×10 mm wafers were used for electrode fabrication. To achieve Ohmic contact with the back side of the wafer, samples were dip-etched in 5% HF, and 50 nm Al and 25 nm Au metal layers were deposited using an Angstrom Engineering EvoVac e-beam evaporator. Copper wires were then connected to the Au layer using silver paste (PECLO®, 16062, Ted Pella). The paste was pre-dried at room temperature for 30 min and fully dried for 30 min at 65° C. The resistance of the interconnects was under 3Ω. Wring and sides of the wafer were insulated, and the glass tube was attached to the electrode using electrical epoxy (8332, MG Chemicals) to improve mechanical stability. The epoxy was cured for 30 min at 65° C. before measurements. Schematics and representative pictures of the electrodes are shown in FIG. 34.

Electrochemical tests. Cyclic voltammetry (CV) and electrochemical impedance spectroscopy (EIS) were performed using a potentiostat (SP-200, BioLogic) controlled with EC-Lab software. A platinum wire was used as the counter electrode, an Ag/AgCI electrode (saturated KCl) as the reference electrode, and a silicon wafer as the working electrode. The potentials were corrected to standard hydrogen electrode potential using E_{Ag/Cl(3.5 M)}=−0.2037 V⁴⁷. Light was delivered from the bottom of the setup using a 532 nm Thorlabs LED. 0.5 M K2SO4 (ReagentPlus®, >99%, P0772-250G) was used as an electrolyte. A pyrex glass dish was used as a cell container. Cell temperature was not controlled and was measured to be within 24-26° C. which was unaffected by the LED illumination. EIS was performed in potentiostatic mode at the open circuit voltage with a sinusoidal voltage amplitude of 25 mV. CV was measured at 20 mV/s and the second CV cycle was plotted. Schematic and pictures of the photoelectrochemistry measurement setup are shown in FIG. 34. EIS data was fitted to electrical circuit models using ZFit solver included in EC-Lab software (V11.43) in the range of 1 Hz-10 kHz.

Silicon membrane photocurrent stability. For long term stability, silicon membranes were stored in 1×PBS at 37° C. in a tissue culture incubator and removed only for measurements in the patch-clamp setup. For short term photostability tests, the membranes were stored in 1×PBS at room temperature in the dark or in the patch-clamp setup under pulsed illumination at 4 Hz.

### Atomic Layer Deposition

Atomic layer deposition (ALD) of TiO₂/Al₂O₃ superlattice was performed on the 5 mm×5 mm stain-etched silicon wafers using Savannah G2 Thermal ALD System. Prior to the ALD process the samples were mounted on the carrier wafer using copper tapes. The chamber has been stabilized at 150° C. for 10 mins before deposition. The flow rate was set to 90 sccm. The TiO₂ deposition cycle comprised alternative pulses of O₃/H₂O (0.1 s, wait 10 s) and TDMAT (Ti precursor) (0.015 s, wait 10 s) for expected deposition rate of 0.6 Å/cycle. The Al₂O₃ deposition cycle comprised alternative pulses of O₃/H₂O (0.015 s, wait 5 s) and TMA (Al precursor) (0.015 s, wait 5 s) for the expected deposition of 1 Å/cycle. To achieve a total deposition profile of 20-cycle TiO₂/4-cycle Al₂O₃, every 1-cycle Al₂O₃ was deposited following every 4-cycle TiO₂ where the process was repeated 5 times.

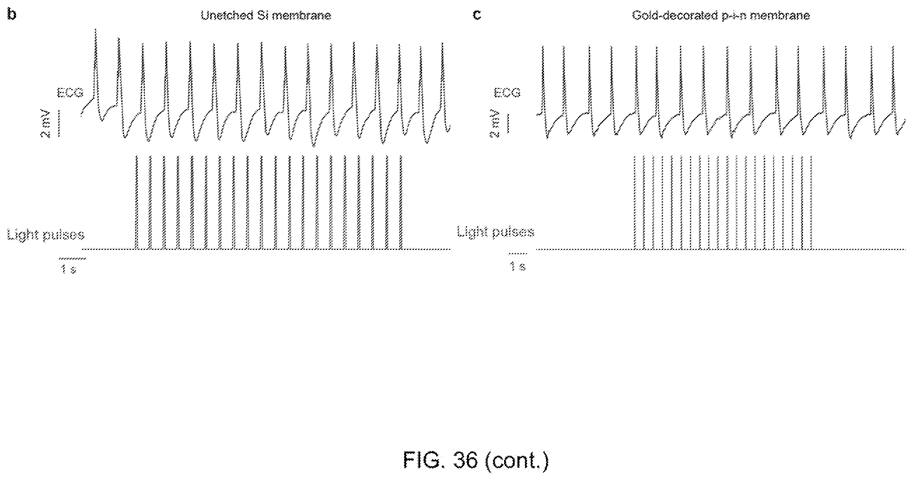

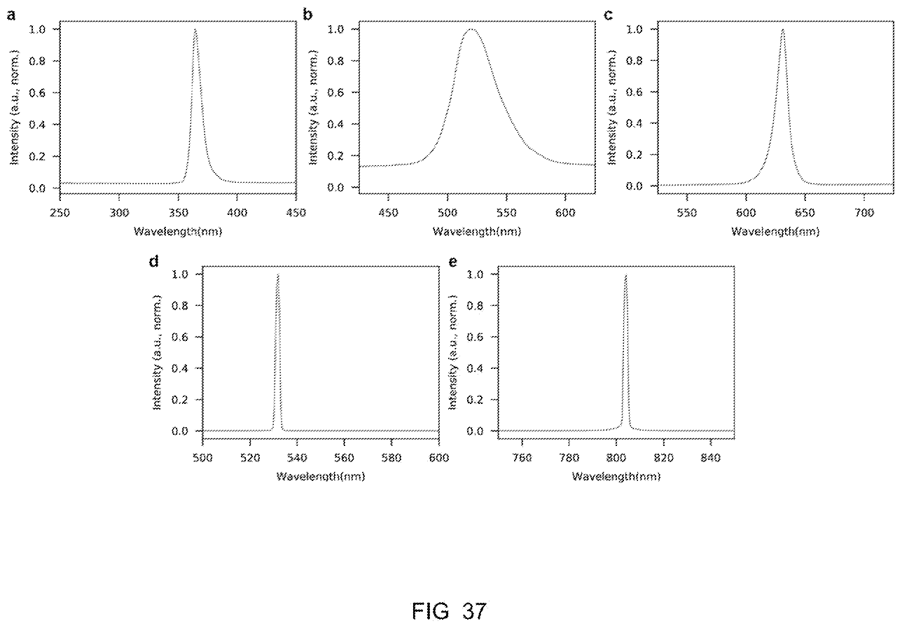

Light sources and light analysis. Light sources used in this study are: 1) 532 nm LED (M530L3 mounted on SM1P25-A, Thorlabs), 2) 365 nm LED (M365L3-C1, Thorlabs), 3) 625 nm LED (M625L4-C1, Thorlabs), 4) diode-pulsed solid-state 532 nm laser (500 mW, LRS-0532-PFM-00300-05, Laserglow) coupled into P1-460AR-2 fiber (Thorlabs), 5) 808 nm diode laser (10 W, LRD-0808-PFI-10000-01, Laserglow) coupled into MHP365L02 fiber (Thorlabs). LEDs were mounted into the microscope and electrochemical setup as received. The 532 nm laser was focused using a CFC-2X-A collimator (Thorlabs) for biostimulation experiments. The 808 nm laser was focused using a F220SMA-850 or F230SMA-850 collimator (Thorlabs) for biostimulation experiments and a F220SMA-85 for microscope setup. Laser power was controlled using front-panel analog knob adjustments and modulated with TTL or analog input signals. Light power was measured using Thorlabs S120C and S310C sensors connected to the PM100D console. Spot sizes for LEDs and lasers were measured using the knife-edge technique (5/95 width). Output spectra of the light sources were measured using a Fergie spectrometer (Princeton Instruments) and are displayed in FIG. 37.

Scanning electron microscopy was performed using a Merlin scope (Carl Zeiss). Samples were mounted on aluminum sample holders using copper tape. Cross sections were prepared by scratching the back side of the wafer using diamond scribe, breaking the sample in half and mounting on a 90° sample holder using copper tape. EDS was performed on the same scope using an Oxford Ultim Max 100 EDS system and data were analyzed using AZTEC software (Oxford Instruments). Raw images with attached metadata are available in an online repository.

Focused ion beam milling. Samples for scanning transmission electron microscopy (STEM) were prepared using the Tescan LYRA3 FIB-SEM system equipped with a gallium ion gun. The lamella was lifted in situ with a tungsten manipulator and transferred to the post on the copper TEM half-circle grid. A platinum layer (˜1 μm) was deposited onto sample to protect its surface during milling to the thickness of ˜200 nm. SEM images of the lamella are shown in FIG. 29.

Scanning transmission electron microscopy (STEM) was carried out using a 200 kV aberration-corrected JEOL ARM200F with a cold field emission source, which gives spatial resolution ˜0.8 Å. High-angle annular dark field (HAADF) detector angle was set to 90-270 mrad to give Z contrast images. Diffraction patterns were taken under TEM mode. Images were processed using ImageJ software. Raw images displaying all recording parameters are available in an online repository.

Atomic force microscopy (AFM) was performed with a Bruker Dimension Icon microscope using a ScanAsyst probe in automated Peak Force Tapping™ mode. Gwyddion software was used to remove scars from the scans, crop the scan area, and plot the 3D model of the material surface. Raw data is available in an online repository.

### Indentation

Modulus and hardness measurements were carried out using a T1950 Triboindenter (Bruker-Hysitron) with a Berkovich tip at Scanned Probe Imaging and Development facility at NUANCE center, Northwestern University. The loading-holding-unloading cycles were used for the nanoindentations test. Standard quartz substrate was used to calibrate the system before measurement on silicon samples. Indentation results obtained were analysed based on the method suggested by Oliver and Pharr48. Raw data and calculation summaries are available in an online repository.

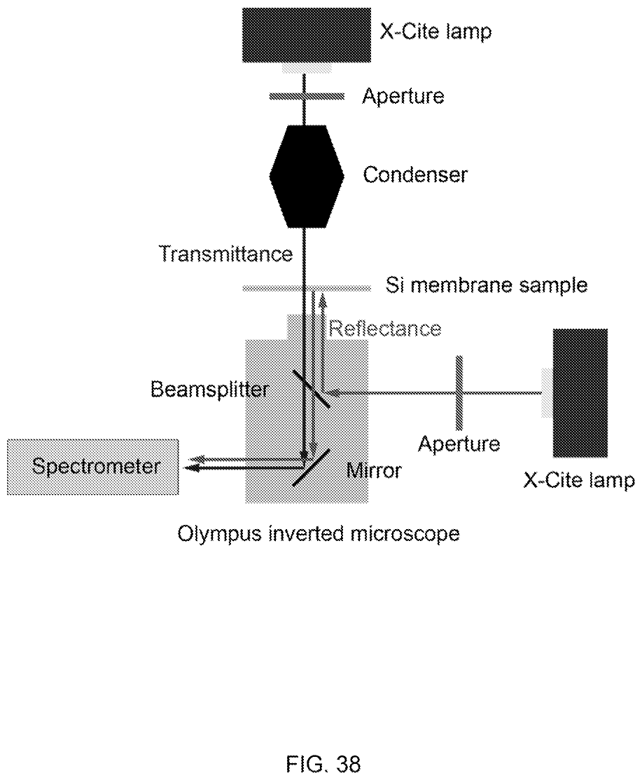

Silicon membrane microspectrometry was performed on an inverted microscope (Olympus IX71) using a 20×/0.45 Phi (Olympus) lens. A fluorescent X-Cite lamp (120PC Q, Lumen Dynamics) was used as a light source in both transmission and reflectance mode. The spot size was reduced to <1 mm using the input aperture. An 80/20 beamsplitter (21001, Chroma) was used for reflectance measurements. Spectra were recorded using a Fergie spectrometer and LightField software (Princeton Instruments). A dielectric mirror (BB1-E02, Thorlabs) was used as a reflectance standard. The presented spectra are an average of at least six measurements from different membranes to average the effects of thin-film interference on the measurements. For transmittance, PDMS absorption was subtracted. Schematic of the experimental setup is shown in FIG. 38.

Optical materials microscopy. Color optical microscopy was performed on the Olympus OLS5000 LEXT system.

Fluorescence measurements were performed on a HORIBA Fluorolog-3 Spectrofluorometer using 320 nm excitation light and a Synapse OE-CCD detector.

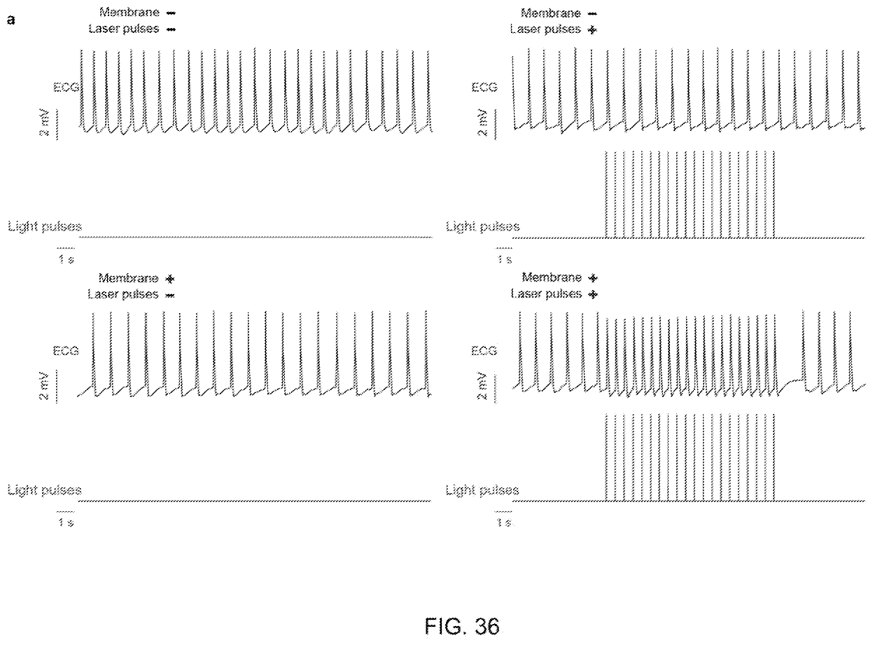

Ex vivo heart stimulation. Isolated heart preparation followed methods described previously²⁹. In short, an adult rat (males, 300-400 g body weight) was heparinized and anaesthetized using open-drop exposure of isoflurane in a bell jar configuration. The heart was removed and placed in ice cold HBSS buffer, and the aorta was cannulated in preparation for use in a Langendorff set-up. Oxygenated HEPES-buffered Tyrode's solution was perfused through the cannulated aorta after passing through a heating coil and bubble trap (Radnoti). The heart was placed in a water-jacketed beaker (Fisher Scientific) to maintain a temperature of 37° C. The perfusion pressure was maintained at 80-100 mmHg. The sinoatrial node along with the atria were removed to lower atrioventricular node pace. The perfusion and left ventricular pressure (LVP) were monitored using a BP-100 probe (iWorx) connected to the perfusion line and a water-filled balloon (Radnoti) inserted into the left ventricle, respectively. For ECG recordings, needle electrodes were positioned on the left ventricular wall and aorta, ground on the cannula, and connected to a C-ISO-256 preamplifier (iWorx). All signals (perfusion, LVP, and ECG) were amplified using an IA-400D amplifier (iWorx) and interfaced with a computer using a Digidata 1550 digitizer with Clampex software (Molecular Devices). The silicon membrane was placed on the left ventricular wall and adhered stably to the heart. The lasers were pointed at the membranes and stimulation and recording were controlled using the Digidata 1550 digitizer. When only light pulses, or only membranes were used in the experiment, no stimulation was observed. Heart could be paced only when both membranes and light pulses were present (FIG. 36). For dual chamber pacing experiments, hexagon shaped membranes were placed on left and right ventricular wall and light was delivered from 532 nm and 808 nm laser through 200 μm core glass fiber cannulas (CFMC22L20, Thorlabs) coupled to a 200 μm multimode fiber (M86L01 and M84L01, Thorlabs) using ceramic mating sleeve. Electrical mapping was performed using 4×4 micro electrode array (0.7 mm spacing between electrodes) and recorded using Intantech RHD USB interface board and RHD 16-channel input recording headstage. The signals were recorded at 10 kS/s in the 0.1-100 Hz bandwidth. Isochrone maps of the electrical propagation were calculated using Python scripts available in the online repository. Timestamp of peak positive signal deflection for each contraction was determined and average was calculated for multiple signals. Gaussian interpolation was used for map rendering to improve readability.

Acute sciatic nerve stimulation. Sciatic nerve surgery followed methods described previously29. In short, adult rat (10-24 weeks, males and females) was deeply anaesthetized with isoflurane (3-4%). The fur was removed from the hindquarters using a surgical clippers and hair removal cream. An incision across the midline was made in the skin, and the fascial plane was opened between the gluteus maximus and the anterior head of the biceps femoris exposing the sciatic nerve. The nerve was extruded using sutures and the silicon membrane was wrapped around the nerve. For electromyography (EMG) recordings, stainless steel needle electrodes in an ungrounded configuration were inserted into the soleus and connected to a C-ISO-256 preamplifier (iWorx). The EMG signal was amplified using an IA-400D amplifier (iWorx) and interfaced with a computer using a Digidata 1550 digitizer with Clampex software (Molecular Devices). The lasers were pointed at the membranes and stimulation and recording were controlled using the Digidata 1550 digitizer.

Note that the data in FIGS. 6d and e was collected from the same experiment (by only changing the laser source), while the data in f was collected from a different experiment which accounts for the difference in recorded amplitudes. For analysis of leg displacement, a protractor was placed under the leg for a distance reference. Videos of limb movement were recorded using a Sony α6100 camera with a 30 mm macro lens (E 3.5/30, Sony) at 60 frames per second. The videos were cropped and sliced using Adobe Premiere Pro, exported as an image sequence, and imported into ImageJ for processing. The image sequence was set as an 8-bit greyscale stack. A Gaussian filter with 4 px radius was applied to the entire stack and the intensity of the first frame was subtracted. A binary threshold was used to isolate the displacement distance and the maximum displacement was measured under the first nail. If no movement was detected, a value of 0 was assigned to the pulse. Intantech RHD USB interface board and RHD 16-channel bipolar-input recording headstage were used for multichannel EMG recording. The signals were recorded at 10 kS/s in the 0.1-200 Hz bandwidth. Parts of the skin were removed to expose the muscles and silver wires were inserted into gastrocnemius medialis (GM), plantar interossei (PL) and tibialis anterior (TA) muscles following the anatomical cues. Ground silver electrode was placed under the skin on the upper right body side of the animal, opposite of the recording side. Rectangular silicon membrane was placed on the exposed sciatic nerve and was illuminated from above using 532 nm laser light pulses with a spot size of 0.5 mm. The laser collimator was installed on the manual linear translational stage, and the nerve was scanned along the diameter at the steps of 0.1 mm. 12 light pulses were delivered at each position and EMG signals were recorded. Analysis of the recording was performed using Python analysis scripts available in an online repository. In short, for each laser pulse maximum positive deflection of EMG signal was calculated. The selectivity index for each laser position was calculated as suggested in the literature44 using equation

$${SI}_{n} = \frac{E M {G}_{n , norm}}{{Σ}_{i} E M {G}_{i , norm}} ,$$

where SIₙ is a selectivity index for a muscle n, EMG_{n,norm} is a normalized maximum positive signal deflection for muscle n, and ΣᵢEMG_{i, norm} is the sum of normalized maximum positive deflections for all muscles included in the analysis. Signal from PL muscle was background corrected using iterative polynomial smoothing algorithm implemented in pybaselines package to remove large drift coming from the limb movement. No activation was defined where no signals were observed. For acute stimulation with a closed muscle, 532 nm laser pulses were delivered using 200 μm core glass fiber cannulas (CFMC22L20, Thorlabs) coupled to a 200 μm multimode fiber (M86L01, Thorlabs) using ceramic mating sleeve. The muscle was closed using Nylon sutures.

Animal subjects. CD/SD rats were originally obtained from Charles River and were housed and bred in the animal facility at the University of Chicago. The animal room was maintained at a humidity of 40-60% and a temperature of 18-23° C. under a 12-h-light/12-h-dark cycle. The animals were allowed free access to food and water.

Numerical data processing and statistics. Analysis of numerical data and plotting was performed with Python scripts using NumPy, Matplotlib, Scipy, and Seaborn libraries. Statistics were calculated using statmodels library by applying Tukey's honestly significant difference (HSD) multiple comparison test.

Various exemplary embodiments of the disclosure include, but are not limited to the enumerated embodiments listed below, which can be combined in any number and in any combination that is not technically or logically inconsistent.

Embodiment 1. A device comprising a p-type silicon material comprising a nanoporous silicon layer and a nonporous silicon layer that form a heterojunction; and a flexible substrate comprising one or more of polymers on which the p-type silicon material is distributed such that the flexible substrate is in contact with the nonporous silicon layer.

Embodiment 2. The device of embodiment 1, wherein the p-type silicon material is oxygen (O₂) plasma-treated p-type silicon material.

Embodiment 3. The device of embodiment 1 or embodiment 2, wherein the polymer is selected from a biocompatible polymer, a biodegradable polymer, an extracellular matrix protein, and a combination thereof.

Embodiment 4. The device of embodiment 3, wherein the polymer is polydimethylsiloxane, poly(methyl methacrylate), poly lactic-co-glycolic acid, poly(ethylene glycol) diacrylate, collagen, or gelatin.

Embodiment 5. The device of embodiment 3, wherein the flexible substrate is polydimethylsiloxane substrate.

Embodiment 6. The device of any of embodiments 1 to 5, wherein the flexible substrate has an open porosity of at least about 10% (such as, e.g., at least about 30%); or wherein the flexible substrate is non-porous.

Embodiment 7. The device of any of embodiments 1 to 6, wherein the nanoporous silicon layer is mesoporous (i.e., having a pore size in the range of 2 nm and 50 nm).

Embodiment 8. The device of any of embodiments 1 to 7, wherein the nanoporous silicon layer comprises pores having cavities and/or channels.

Embodiment 9. The device of any of embodiments 1 to 7, wherein the nanoporous silicon layer has an average thickness in a range of about 500 nm to 3 μm (such as about 700 nm to 1.5 μm, or about 800 nm to 1.2 μm, or about 1 μm).

Embodiment 10. A method for modulating activity of a cell, the method comprising:
contacting a membrane of the cell with a device according to any one of embodiments 1 to 9 to form a device-cell membrane interface; and
exposing the interface to light under conditions to depolarize the cell membrane thereby increase a threshold for activation of the cell, wherein the cell is capable of being activated by photoelectrochemical effect.

Embodiment 11. The method according to embodiment 10, wherein the cell is a cardiomyocyte, a neuron, or a retinal cell.

Embodiment 12. The method according to embodiment 10, wherein the cell is a cardiomyocyte.

Embodiment 13. The method according to embodiment 10, wherein the cell is a neuron.

Embodiment 14. The method according to any of embodiments 10 to 13, wherein the device-cell interface is a direct interface between the device and the cell membrane, optionally wherein the contacting is without penetrating the cell membrane.

Embodiment 15. A method of treating a disease in a subject by modulating activation of a cell, the method comprising:
providing one or more devices according to any one of embodiments 1 to 9 to the affected cell(s) in the subject; and
exposing the one or more devices to the light under conditions sufficient to overcome a threshold for activation of the cell(s) and treat the disease.

Embodiment 16. The method of embodiment 15, wherein the disease is a cardiovascular disease.

Embodiment 17. The method of embodiment 15, wherein the disease is a neuronal disease or a neuromuscular disease.

Embodiment 18. The method of embodiment 15, wherein the disease is an ophthalmic disease.

Embodiment 19. The method of embodiment 15, wherein the disease is associated with sciatic nerve, such as pain, spinal cord injuries, osteoporosis, urinary and/or bowel incontinence.

Embodiment 20. The method of any of embodiments 10 to 19, wherein the light pulse has a frequency ranging from 0.25 Hz to 50 Hz, such as ranging from 1 Hz to 5 Hz, or about 4 Hz.

Embodiment 21. A method for photoelectrochemically training myocardium in a subject to beat at a target frequency, the method comprising:
contacting the myocardium with one or more devices according to any one of embodiments 1 to 9; and
operating a light emitter to provide, during a training period of time, a plurality of light pulses to the myocardium at the target frequency.

Embodiment 22. The method of embodiment 21, wherein the device is configured to be placed in contact with cells of the myocardium such that the nanoporous silicon layer is in contact with cells of the myocardium.

Embodiment 23. The method of embodiment 21 or embodiment 22, further comprising detecting a pulse rate of the myocardium during a detection period of time, wherein the detection period of time differs from the training period of time.

Embodiment 24. The method of embodiment 23, wherein the detection period of time is subsequent to the training period of time, and wherein the method further comprises:
responsive to the detected pulse rate differing from the target pulse rate by more than a threshold amount, operating the light emitter to provide, during an additional training period of time, an additional plurality of light pulses to the myocardium at the target frequency.

Embodiment 25. A method for photoelectrochemically controlling limb movement in a subject, the method comprising:
contacting sciatic nerve with one or more devices according to any one of embodiments 1 to 9; and
operating a light emitter to provide, during a period of time, a plurality of light pulses to the sciatic nerve at the target frequency.

Embodiment 26. The method of embodiment 25, wherein the device is configured to be placed in contact with sciatic nerve cells such that the nanoporous silicon layer is in contact with sciatic nerve cells.

Embodiment 27. The method of any of embodiments 10 to 26, wherein the light is provided at an excitation wavelength ranging from 400 to 900 nm.

Embodiment 28. The method of any of embodiments 10 to 27, wherein the light is provided at a power in a range of 0.1 mW/mm² to 20 mW/mm², such as 2 mW/mm² to 10 mW/mm².

Embodiment 29. A system for treating a disease in a subject by modulating activation of a cell, the system comprising:
one or more devices according to any one of embodiments 1 to 9;
a light emitter configured to provide a light pulse to the device, wherein the one or more devices provide, to the cell they are in contact with, excitatory stimulus in response to receiving the light; and
a controller that is operably coupled to the light source, wherein the controller comprises one or more processors, wherein the controller is programmed to perform controller operations including: operating the light source to provide the light pulse to the cell.

Embodiment 30. The system of embodiment 29 for electrochemically training myocardium to beat at a target frequency.

Embodiment 31. The system of embodiment 29 or embodiment 30, wherein the device is configured to be placed in contact with cells of the myocardium such that the nanoporous silicon layer is in contact with cells of the myocardium.

Embodiment 32. The system of embodiment 31, wherein the device is configured to be placed in contact with sciatic nerve cells such that the nanoporous silicon layer is in contact with sciatic nerve cells.

Embodiment 33. The system of any of embodiments 29 to 32, wherein the light is provided at an excitation wavelength ranging from 400 to 900 nm.

Embodiment 34. The method of embodiment 33, wherein the light is provided at a power in a range of 0.1 mW/mm² to 20 mW/mm², such as 2 mW/mm² to 10 mW/mm².

Embodiment 35. A method for modulating activity of an engineered tissue, the method comprising: contacting the engineered tissue surface with the device of any of embodiments 1-9 to form a device-engineered tissue interface; and exposing the interface to light under conditions to depolarize the cell membrane thereby increase a threshold for activation of the engineered tissue, wherein the engineered tissue are capable of being activated by light.

### REFERENCES

1 Feiner, R. et al. Engineered hybrid cardiac patches with multifunctional electronics for online monitoring and regulation of tissue function. Nature Materials 15, 679-685, doi:10.1038/nmat4590 (2016).
2 Fang, H. et al. Capacitively Coupled Arrays of Multiplexed Flexible Silicon Transistors for Long-Term Cardiac Electrophysiology. Nat Biomed Eng 1, 1-12, doi:10.1038/s41551-017-0038 (2017).
3 Xu, L. et al. 3D multifunctional integumentary membranes for spatiotemporal cardiac measurements and stimulation across the entire epicardium. Nat Commun 5, 3329, doi:10.1038/ncomms4329 (2014).
4 Kim, D. H. et al. Epidermal electronics. Science 333, 838-843, doi:10.1126/science.1206157 (2011).
5 Jiang, Y. et al. Rational design of silicon structures for optically controlled multiscale biointerfaces. Nat Biomed Eng 2, 508-521, doi:10.1038/s41551-018-0230-1 (2018).
6 Feiner, R. & Dvir, T. Tissue-electronics interfaces: from implantable devices to engineered tissues. Nature Reviews Materials 3, 1-16 (2017).
7 Murphy, J. J. Current practice and complications of temporary transvenous cardiac pacing. BMJ 312, 1134, doi:10.1136/bmj.312.7039.1134 (1996).
8 Austin, J. L., Preis, L. K., Crampton, R. S., Beller, G. A. & Martin, R. P. Analysis of pacemaker malfunction and complications of temporary pacing in the coronary care unit. The American journal of cardiology 49, 301-306, doi:10.1016/0002-9149(82)90505-7 (1982).
9 Betts, T. R. Regional survey of temporary transvenous pacing procedures and complications. Postgraduate medical journal79, 463-465, doi:10.1136/pmj.79.934.463 (2003).
10 Nolewajka, A. J., Goddard, M. D. & Brown, T. C. Temporary transvenous pacing and femoral vein thrombosis. Circulation 62, 646-650, doi:10.1161/01.cir.62.3.646 (1980).
11 Rossillo, A. et al. Impact of coronary sinus lead position on biventricular pacing: mortality and echocardiographic evaluation during long-term follow-up. Journal of cardiovascular electrophysiology 15, 1120-1125, doi:10.1046/j.1540-8167.2004.04089.x (2004).
12 Peschar, M., de Swart, H., Michels, K. J., Reneman, R. S. & Prinzen, F. W. Left ventricular septal and apex pacing for optimal pump function in canine hearts. J Am Coll Cardiol 41, 1218-1226, doi:10.1016/s0735-1097(03)00091-3 (2003).
13 Wells, J. et al. Optical stimulation of neural tissue in vivo. Opt Lett 30, 504-506, doi:10.1364/ol.30.000504 (2005).
14 Wells, J., Konrad, P., Kao, C., Jansen, E. D. & Mahadevan-Jansen, A. Pulsed laser versus electrical energy for peripheral nerve stimulation. J Neurosci Methods 163, 326-337, doi:10.1016/j.jneumeth.2007.03.016 (2007).
15 Jenkins, M. W. et al. Optical pacing of the embryonic heart. Nat Photonics 4, 623-626, doi:10.1038/nphoton.2010.166 (2010).
16 Jenkins, M. W. et al. Optical pacing of the adult rabbit heart. Biomed Opt Express 4, 1626-1635, doi:10.1364/BOE.4.001626 (2013).
17 McCall, J. G. et al. Preparation and implementation of optofluidic neural probes for in vivo wireless pharmacology and optogenetics. Nat Protoc 12, 219-237, doi:10.1038/nprot.2016.155 (2017).
18 Montgomery, K. L. et al. Wirelessly powered, fully internal optogenetics for brain, spinal and peripheral circuits in mice. Nat Methods 12, 969-974, doi:10.1038/nmeth.3536 (2015).
19 Nussinovitch, U. & Gepstein, L. Optogenetics for in vivo cardiac pacing and resynchronization therapies. Nature Biotechnology 33, 750-754, doi:10.1038/nbt.3268 (2015).
Koo, J. et al. Wireless bioresorbable electronic system enables sustained nonpharmacological neuroregenerative therapy. Nat Med 24, 1830-1836, doi:10.1038/s41591-018-0196-2 (2018).
21 Choi, Y. S. et al. Fully implantable and bioresorbable cardiac pacemakers without leads or batteries. Nat Biotechnol 39, 1228-1238, doi:10.1038/s41587-021-00948-x (2021).
22 Piech, D. K. et al. A wireless millimetre-scale implantable neural stimulator with ultrasonically powered bidirectional communication. Nat Biomed Eng 4, 207-222, doi:10.1038/s41551-020-0518-9 (2020).
23 Jiang, Y. & Tian, B. Inorganic semiconductor biointerfaces. Nat Rev Mater 3, 473-490, doi:10.1038/s41578-018-0062-3 (2018).
24 Rotenberg, M. Y. & Tian, B. Talking to cells: semiconductor nanomaterials at the cellular interface. Advanced Biosystems 2, 1700242 (2018).
25 Silvera Ejneby, M. et al. Chronic electrical stimulation of peripheral nerves via deep-red light transduced by an implanted organic photocapacitor. Nat Biomed Eng, doi:10.1038/s41551-021-00817-7 (2021).
26 Yan, F., Bao, X.-M. & Gao, T. Photovoltage spectra of silicon/porous silicon heterojunction. Solid State Communications 91, 341-343, doi:10.1016/0038-1098(94)90630-0 (1994).
27 Palsule, C. et al. Electrical and optical characterization of crystalline silicon/porous silicon heterojunctions. Solar Energy Materials and Solar Cells 46, 261-269, doi:10.1016/s0927-0248(97)00004-4 (1997).
28 Suntao, W., Yanhua, W. & Qihua, S. Measurement and analysis of the characteristic parameters for the porous silicon/silicon using photovoltage spectra. Applied Surface Science 158, 268-274, doi:10.1016/s0169-4332(00)00008-8 (2000).
29 Fang, Y. et al. Micelle-enabled self-assembly of porous and monolithic carbon membranes for bioelectronic interfaces. Nat Nanotechnol 16, 206-213, doi:10.1038/s41565-020-00805-z (2021).
30 Jiang, Y. et al. Heterogeneous silicon mesostructures for lipid-supported bioelectric interfaces. Nat Mater 15, 1023-1030, doi:10.1038/nmat4673 (2016).
31 Kolasinski, K. W. in Handbook of Porous Silicon Ch. Chapter 4, 39-59 (2018).
32 Yerokhov, V. Y. & Melnyk, I. I. Porous silicon in solar cell structures: a review of achievements and modern directions of further use. Renewable and Sustainable Energy Reviews 3, 291-322, doi:10.1016/s1364-0321(99)00005-2 (1999).
33 Alhmoud, H., Brodoceanu, D., Elnathan, R., Kraus, T. & Voelcker, N. H. A MACEing silicon: Towards single-step etching of defined porous nanostructures for biomedicine. Progress in Materials Science 116, 100636, doi:10.1016/j.pmatsci.2019.100636 (2021).
34 Hopcroft, M. A., Nix, W. D. & Kenny, T. W. What is the Young's Modulus of Silicon?Journal of Microelectromechanical Systems 19, 229-238, doi:10.1109/jmems.2009.2039697 (2010).
35 Jiang, Y. et al. Nongenetic optical neuromodulation with silicon-based materials. Nat Protoc 14, 1339-1376, doi:10.1038/s41596-019-0135-9 (2019).
36 Glunz, S. W. & Feldmann, F. SiO2 surface passivation layers—a key technology for silicon solar cells. Solar Energy Materials and Solar Cells 185, 260-269, doi:10.1016/j.solmat.2018.04.029 (2018).
37 Nosaka, Y. & Nosaka, A. Y. Generation and Detection of Reactive Oxygen Species in Photocatalysis. Chem Rev 117, 11302-11336, doi:10.1021/acs.chemrev.7b00161 (2017).
38 Tampo, H., Kim, S., Nagai, T., Shibata, H. & Niki, S. Improving the Open Circuit Voltage through Surface Oxygen Plasma Treatment and 11.7% Efficient Cu2ZnSnSe4 Solar Cell. ACS Appl Mater Interfaces 11, 13319-13325, doi:10.1021/acsami.9b01756 (2019).
39 Parameswaran, R. et al. Optical stimulation of cardiac cells with a polymer-supported silicon nanowire matrix. Proc Natl Acad Sci USA 116, 413-421, doi:10.1073/pnas.1816428115 (2019).
40 Bruegmann, T. et al. Optogenetic control of heart muscle in vitro and in vivo. Nat Methods 7, 897-900, doi:10.1038/nmeth.1512 (2010).
41 Jacques, S. L. Optical properties of biological tissues: a review. Phys Med Biol 58, R37-61, doi:10.1088/0031-9155/58/11/R37 (2013).
42 Ushiki, T. & Ide, C. Three-dimensional organization of the collagen fibrils in the rat sciatic nerve as revealed by transmission-and scanning electron microscopy. Cell and tissue research 260, 175-184 (1990).
43 Koutsou, A. D., Moreno, J. C., del Ama, A. J., Rocon, E. & Pons, J. L. Advances in selective activation of muscles for non-invasive motor neuroprostheses. Journal of NeuroEngineering and Rehabilitation 13, 56, doi:10.1186/s12984-016-0165-2 (2016).
44 Badia, J. et al. Comparative analysis of transverse intrafascicular multichannel, longitudinal intrafascicular and multipolar cuff electrodes for the selective stimulation of nerve fascicles. J Neural Eng 8, 036023, doi:10.1088/1741-2560/8/3/036023 (2011).
45 Strauss, I. et al. Q-PINE: A quick to implant peripheral intraneural electrode. J Neural Eng 17, 066008, doi:10.1088/1741-2552/abc52a (2020).
46 Guo, J. et al. Highly Stretchable, Strain Sensing Hydrogel Optical Fibers. Adv Mater 28, 10244-10249, doi:10.1002/adma.201603160 (2016).
47 Kahlert, H. in Electroanalytical Methods (eds Fritz Scholz et al.) Ch. Chapter 15, 291-308 (Springer Berlin Heidelberg, 2010).
48 Oliver, W. C. & Pharr, G. M. An improved technique for determining hardness and elastic modulus using load and displacement sensing indentation experiments. Journal of Materials Research 7, 1564-1583, doi:10.1557/jmr.1992.1564 (1992).

1. A device comprising a p-type semiconductor material comprising a nanoporous semiconductor layer and a nonporous semiconductor layer that form a heterojunction; and a flexible substrate comprising one or more of polymers on which the p-type semiconductor material is distributed such that the flexible substrate is in contact with the nonporous semiconductor layer.

2. The device of claim 1, wherein the semiconductor material is silicon, silicon carbide, gallium nitride, gallium arsenide, indium phosphide, cadmium sulfide, cadmium selenide, or cadmium telluride.

3. The device of claim 1, wherein the semiconductor material is silicon.

4. The device of claim 1, wherein the p-type semiconductor material is oxygen (O₂) plasma-treated p-type semiconductor material.

5. The device of claim 4, wherein the p-type semiconductor material is p-type silicon.

6. The device of claim 1, wherein the polymer is selected from a biocompatible polymer, a biodegradable polymer, an extracellular matrix protein, and a combination thereof.

7. The device of claim 6, wherein the polymer is polydimethylsiloxane, poly(methyl methacrylate), poly lactic-co-glycolic acid, poly(ethylene glycol) diacrylate, collagen, or gelatin.

8. The device of claim 6, wherein the flexible substrate is polydimethylsiloxane substrate.

9. The device of claim 1, wherein the flexible substrate has an open porosity of at least about 10%, or at least about 30%; or wherein the flexible substrate is non-porous.

10. The device of claim 1, wherein the nanoporous semiconductor layer is mesoporous or having a pore size in the range of 2 nm to 50 nm.

11. The device of claim 1, wherein the nanoporous semiconductor layer comprises pores having cavities and/or channels.

12. The device of claim 1, wherein the nanoporous semiconductor layer has an average thickness in a range of about 500 nm to 3 μm, about 700 nm to 1.5 μm, about 800 nm to 1.2 μm, or about 1 μm.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. A method for photoelectrochemically training myocardium in a subject to beat at a target frequency, the method comprising:
contacting the myocardium with one or more devices according to claim 1; and
operating a light emitter to provide, during a training period of time, a plurality of light pulses to the myocardium at the target frequency.

25. The method of claim 24, wherein the device is configured to be placed in contact with cells of the myocardium such that the nanoporous semiconductor layer is in contact with cells of the myocardium.

26. The method of claim 24, further comprising detecting a pulse rate of the myocardium during a detection period of time, wherein the detection period of time differs from the training period of time.

27. The method of claim 26, wherein the detection period of time is subsequent to the training period of time, and wherein the method further comprises:
responsive to the detected pulse rate differing from the target pulse rate by more than a threshold amount, operating the light emitter to provide, during an additional training period of time, an additional plurality of light pulses to the myocardium at the target frequency.

28. (canceled)

29. (canceled)

30. (canceled)

31. (canceled)

32. A system for treating a disease in a subject by modulating activation of a cell, the system comprising:
one or more devices according to claim 1;
a light emitter configured to provide a light pulse to the device, wherein the one or more devices provide, to the cell they are in contact with, excitatory stimulus in response to receiving the light; and
a controller that is operably coupled to the light source, wherein the controller comprises one or more processors, wherein the controller is programmed to perform controller operations including: operating the light source to provide the light pulse to the cell.

33. The system of claim 32, for electrochemically training myocardium to beat at a target frequency.

34. The system of claim 32, wherein the device is configured to be placed in contact with cells of the myocardium such that the nanoporous semiconductor layer is in contact with cells of the myocardium or wherein the device is configured to be placed in contact with sciatic nerve cells such that the nanoporous semiconductor layer is in contact with sciatic nerve cells.

35. (canceled)

36. The system of claim 32, wherein the light is provided at an excitation wavelength ranging from 400 to 900 nm and/or wherein the light is provided at a power in a range of 0.1 mW/mm² to 20 mW/mm², such as 2 mW/mm² to 10 mW/mm².

37. (canceled)
